SEQUENCE LISTING (29 sequences)

SEQ ID NO: 1 - DNA, 52 bp, Artificial Sequence
  Description of Artificial Sequence Synthetic primer
aattctaatacgactcactatagggccaaattggctactaccgaagagctac

SEQ ID NO: 2 - DNA, 52 bp, Artificial Sequence
  Description of Artificial Sequence Synthetic primer
aattctaatacgactcactatagggccaaattggctactaccgtagagctac

SEQ ID NO: 3 - DNA, 27 bp, Artificial Sequence
  Description of Artificial Sequence Synthetic primer
cacagtttgctgtttcttctgtctctg

SEQ ID NO: 4 - DNA, 28 bp, Artificial Sequence
  Description of Artificial Sequence Synthetic primer
cacagtttgttgtttcttctgtctctgc

SEQ ID NO: 5 - DNA, 41 bp, Artificial Sequence
  Description of Artificial Sequence Synthetic oligonucleotide
tgcggccaatgtttgtaatcagccaaggaaattttggggac

SEQ ID NO: 6 - DNA, 39 bp, Artificial Sequence
  Description of Artificial Sequence Synthetic oligonucleotide
cgcattggcatggaagtcactttgatggcacctgtgtag

SEQ ID NO: 7 - DNA, 39 bp, Artificial Sequence
  Description of Artificial Sequence Synthetic oligonucleotide
cgcattggcatggaagtcactttaatggctccatgataa

SEQ ID NO: 8 - DNA, 18 bp, Artificial Sequence
  Description of Artificial Sequence Synthetic oligonucleotide
aacacaagctttcggcag

SEQ ID NO: 9 - DNA, 18 bp, Artificial Sequence
  Description of Artificial Sequence Synthetic oligonucleotide
cactcaagcatttgggag

SEQ ID NO: 10 - DNA, 22 bp, Artificial Sequence
  Description of Artificial Sequence Synthetic oligonucleotide
gaaatttggatctttgtcatcc

SEQ ID NO: 11 - DNA, 22 bp, Artificial Sequence
  Description of Artificial Sequence Synthetic oligonucleotide
gaattgtggatctttgtcatcc

SEQ ID NO: 12 - DNA, 19 bp, Artificial Sequence
  Description of Artificial Sequence Synthetic oligonucleotide
ttccttgtctgattagttc

SEQ ID NO: 13 - DNA, 19 bp, Artificial Sequence
  Description of Artificial Sequence Synthetic oligonucleotide
ttccttgtctgattaattc

SEQ ID NO: 14 - DNA, 18 bp, Artificial Sequence
  Description of Artificial Sequence Synthetic oligonucleotide
accttcgggaacgtggtt

SEQ ID NO: 15 - DNA, 18 bp, Artificial Sequence
  Description of Artificial Sequence Synthetic oligonucleotide
accttcgggaacatggct

SEQ ID NO: 16 - DNA, 18 bp, Artificial Sequence
  Description of Artificial Sequence Synthetic oligonucleotide
aacacaagctttcggcag

SEQ ID NO: 17 - DNA, 18 bp, Artificial Sequence
  Description of Artificial Sequence Synthetic oligonucleotide
cactcaagcatttgggag

SEQ ID NO: 18 - DNA, 20 bp, Artificial Sequence
  Description of Artificial Sequence Synthetic oligonucleotide
ccgaagaacgctgaagcgct

SEQ ID NO: 19 - DNA, 20 bp, Artificial Sequence
  Description of Artificial Sequence Synthetic oligonucleotide
ccaaagaatgcagaggcact

SEQ ID NO: 20 - DNA, 20 bp, Artificial Sequence
  Description of Artificial Sequence Synthetic oligonucleotide
ttcttcggaatgtcgcgcat

SEQ ID NO: 21 - DNA, 20 bp, Artificial Sequence
  Description of Artificial Sequence Synthetic oligonucleotide
ttctttggaatgtcacgcat

SEQ ID NO: 22 - DNA, 20 bp, Artificial Sequence
  Description of Artificial Sequence Synthetic oligonucleotide
cccccagcgcttcagcgttc

SEQ ID NO: 23 - DNA, 20 bp, Artificial Sequence
  Description of Artificial Sequence Synthetic oligonucleotide
ctccaagtgcctctgcattc

SEQ ID NO: 24 - DNA, 21 bp, Artificial Sequence
  Description of Artificial Sequence Synthetic oligonucleotide
cgttaatcgcgtataatacgg

SEQ ID NO: 25 - protein, 6 aa, Artificial Sequence
  Description of Artificial Sequence Synthetic 6xHis tag
HHHHHH

SEQ ID NO: 26 - DNA, 88 bp, Severe acute respiratory syndrome coronavirus 2
ctgattacaaacattggccgcaaattgcacaatttgcccccagcgcttcagcgttcttcggaatgtcgcgcattggcatggaagtcac

SEQ ID NO: 27 - DNA, 88 bp, Unknown
  Description of Unknown bat-SL-CoVZC45 N-gene sequence
ctgattacaaacattggccgcaaattgcacaatttgctccaagtgcctctgcattctttggaatgtcacgcattggcatggaagtcac

SEQ ID NO: 28 - protein, 4 aa, Artificial Sequence
  Description of Artificial Sequence Synthetic peptide
DEAD

SEQ ID NO: 29 - protein, 4 aa, Artificial Sequence
  Description of Artificial Sequence Synthetic peptide
DEAH

(19) United States
(12) Patent Application Publication
(10) Pub. No.: US 2023/0332256 A1
(43) Pub. Date: Oct. 19, 2023

(54) NUCLEIC ACID DETECTION USING A NUCLEASE ACTUATOR

(71) Applicant: The Johns Hopkins University, Baltimore, MD (US)

(72) Inventors: Taekjip Ha, Baltimore, MD (US); Roger Zou, Baltimore, MD (US)

(21) Appl. No.: 18/027,067

(22) PCT Filed: Sep. 17, 2021
(86) PCT No.: PCT/US2021/050962

§ 371 Date: Mar. 17, 2023

Related U.S. Application Data
(60) Provisional application No. 63/079,709, filed on Sep. 17, 2020.

(51) Int. Cl. C12Q 1/70; C12N 9/22; C12Q 1/6853; C12Q 1/6844

(52) U.S. Cl. CPC C12Q 1/701; C12N 9/22; C12Q 1/6853; C12Q 1/6846; C12N 2310/20

(57) ABSTRACT

Identification and rapid specific nucleic acid detection of target nucleic acids includes the steps of target nucleic acid amplification and subjecting the amplified target nucleic acids to sequence specific cleavage.

This application relates to and claims priority from U.S. Patent Application No. 63/079,709 filed on Sep. 17, 2020, the entire disclosure of which is incorporated herein by reference.

### STATEMENT REGARDING FEDERALLY SPONSORED RESEARCH

This invention was made with government support under grant number GM122569 awarded by the National Institutes of Health. The government has certain rights in this invention.

### FIELD

The technology relates in part to methods and compositions for detection and identification of target nucleic acid sequences in a sample. The method includes an isothermal amplification of nucleic acids followed by subjecting the amplified products to nuclease activity as an actuator to allow convenient discrimination between specific and non-specific amplification.

### BACKGROUND

Nucleic acid-based diagnostics can be useful for rapid detection of infection, disease and/or genetic variations. For example, identification of bacterial or viral nucleic acid in a sample can be useful for diagnosing a particular type of infection. Other examples include identification of single nucleotide polymorphisms for disease management or forensics, and identification of genetic variations indicative of genetically modified food products, or identification of agents in the environment e.g. bacteria in a water sample.

### SUMMARY

The disclosure provides for a rapid, inexpensive, sensitive, and specific nucleic acid detection. In general, the method utilizes two main steps: (i) an isothermal amplification step and (ii) subjecting the amplified nucleic acids to sequence specific nucleases.

Accordingly, in certain embodiments a method of detecting a nucleic acid of interest in a sample, comprises preparing primers complementary to the nucleic acid of interest wherein the primers are protected against nuclease activity; performing isothermal amplification to amplify small amounts of either RNA or DNA sequences from the sample to double stranded DNA (dsDNA); subjecting the amplified nucleic acid of interest to a sequence-specific nuclease; and, detecting the nucleic acid of interest.

In certain embodiments, the nuclease activity is inhibited by exonuclease inhibitors comprising: citrate, citrate acid; MES, 2-morpholin-4-ylethanesulfonate; PV6R, pontacyl violet 6R; PCMPS, p-chloromercuriphenyl sulfonate; NCA, 7-nitroindole-2-carboxylic acid; DR396, 4-[(4,6-dichloro-1,3,5 -triazin-2-y0amino]-2-(3 -hydroxy-6-oxoxanthen-9-yl)benzoic acid; DTNB, 5,5′-dithiobis(2-nitrobenzoic acid); ATA, aurintricarboxylic acid; FDCO, fmoc-D-Cha-OH, Mirin, PFM01/SML1735, PFM03, PFM39/SML1839 or combinations thereof.

In certain embodiments, the primers comprise one or more modifications to protect against exonuclease activity. In certain embodiments, the primers comprise phosphorothiolate bonds, secondary structures, polylinkers, fluorescent tags, biotin, affinity labels, reactive groups, 2′-O-modified riboses, inverted dT, inverted, 2′,3′ dideoxy-dT base (5′ Inverted ddT), phosphorylation, phosphoramidite C3 Spacer or combinations thereof.

In certain embodiments, the nuclease activity is inhibited by nuclease inhibitors comprising: diethyl pyrocarbonate, ethanol, aurintricarboxylic acid (ATA), formamide, vanadyl-ribonucleoside complexes, macaloid, ethylenediamine tetraacetic acid (EDTA), proteinase K, heparin, hydroxylamine-oxygen-cupric ion, bentonite, ammonium sulfate, dithiothreitol (DTT), beta-mercaptoethanol, cysteine, dithioerythritol, tris (2-carboxyethyl) phosphene hydrochloride, divalent cations or combinations thereof. In certain embodiments, the divalent cations comprise: Mg²⁺, Mn²⁺, Zn²⁺, Fe²⁺, Ca²⁺, Cu²⁺ or combinations thereof.

In certain embodiments, the isothermally amplified product is split into two equal volumes, a first volume {A} and a second volume {B}. In certain embodiments, a master mix is added to both volumes {A} and {B}. In certain embodiments, the master mix comprises: (1) a sequence-specific nuclease and (2) a double-stranded DNA exonuclease. In certain embodiments, the master mix optionally comprises a helicase. In certain embodiments, the helicase comprises one or more helicases selected from superfamily 1 through 6. In certain embodiments, the helicase comprises RepX, RecQ helicases, MCM 2-7 helicase complexes, Dead-box helicases, DEAH-box helicases or UPF1 like RNA helicases.

Helicases are enzymes capable of unwinding and separating double-stranded nucleic acid into single strands. Examples of helicases include human DNA helicases (and their equivalents in other organisms) such as DNA helicase Q1, Bloom syndrome protein, Werner syndrome protein, DNA helicase Q4, DNA helicase Q5, DNA helicase 2subunit 1, MCM2, MCM3, MCM, MCMS, MCM6, MCM7, MCM8, MCM9, MCM10, Nucleolin, CHD2, CHD7, XPB, XPD, lymphoid-specific helicase, hINO, RuvB-like 1, RuvB-like 2, PIF1, Twinkle, BACH1, RecQ5 alpha, RecQ5 beta, RecQ5 gamma and RTEL1; human RNA helicases (and their equivalents in other organisms) such as RNA helicase DDX1, RNA helicase eIF4A-1, RNA helicase eIF4A-2, RNA helicase DDX3X, RNA helicase DDX3Y, RNA helicaseDDX4, RNA helicase DDXS, RNA helicase DDX6, RNA helicase DHX8, RNA helicase A, RNA helicase DDX10, RNA helicase DDX11, RNA helicase DDX12,Helicase SKI2W, RNA helicase DHX15, RNA helicase DHX16, RNA helicaseDDX17, RNA helicase DDX18, RNA helicase DDX19A, RNA helicase DDX19B, RNA helicase DDX20, Nucleolar RNA helicase 2, RNA helicase DDX23, RNA helicase DDX24, RNA helicase DDX25, RNA helicase DDX27, RNA helicaseDDX28, RNA helicase DHX29, RNA helicase DHX30, RNA helicase DDX31, RNA helicase DHX32, RNA helicase DHX33, RNA helicase DHX34, RNA helicaseDHX35, RNA helicase DHX36, RNA helicase DHX37, RNA helicase PRP 16, RNA helicase DDX39, RNA helicase DHX40, RNA helicase DDX41, RNA helicaseDDX42, RNA helicase DDX43, RNA helicase DDX46, RNA helicase DDX47, RNA helicase eIF4A-3, RNA helicase DDX49, RNA helicase DDX50, RNA helicaseDDX51, RNA helicase DDX52, RNA helicase DDX53, RNA helicase DDX54, RNA helicase DDX55, RNA helicase DDX56, RNA helicase DHX57, RNA helicaseDDX58, RNA helicase DHX58, RNA helicase DDX59, RNA helicase DDX60,Spliceosome RNA helicase BAT1, U5.snRNP 200 kDa helicase, Transcriptional regulator ATRX helicase, RNA helicase SUPV3L1, mitochondrial Superkiller viralicidic activity 2-like 2 (SKIV2L2), and Fanconi anemia group J protein; and commercially available helicases. Amplification conditions that do not include use of a helicase may be referred to herein as helicase-free amplification conditions.

In certain embodiments, a double-stranded DNA exonuclease comprises lambda exonuclease, exonuclease I, exonuclease II, exonuclease III, exonuclease IV, exonuclease V, exonuclease VII or T7 exonuclease. In certain embodiments, a Cas9 guide RNA with sequence complementary to the target nucleic acid sequence of the nucleic acid of interest is added to the first volume. In certain embodiments, a Cas9 guide RNA lacking complementarity to a target nucleic acid sequence is added to the second volume. In certain embodiments, each volume is incubated and DNA concentrations are measured. In certain embodiments, an on-target amplification in the isothermal amplification step results in Cas9 cleavage and exonuclease degradation of the on-target product. In certain embodiments, an off-target amplification product is not cleaved or degraded. In certain embodiments, the nucleic acid of interest is present in a sample as measured by a relative reduction in dsDNA concentration in the first volume versus the second volume.

In certain embodiments, a sequence-specific nuclease comprises Clustered Regularly Interspaced Short Palindromic Repeat (CRISPR)-associated endonucleases or homologs thereof, endonucleases, exo-nucleases, Argonautes, restriction enzymes, zinc-finger nucleases (ZFNs), transcription activator-like effector nucleases (TALENs) meganucleases, or combinations thereof.

In certain embodiments, the CRISPR-associated endonucleases comprise: Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, CjCas9, SpCas9, Cas13, Cas14, Cpf1, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmrl, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, Cu1966 or combinations thereof.

In certain embodiments, the CRISPR-associated endonuclease is guided to the nucleic acid sequence of interest by at least one guide RNA (gRNA) wherein the gRNA is complementary to a target nucleic acid sequence of the nucleic acid of interest. In certain embodiments, the CRISPR-associated endonuclease is an actuator to discriminate between specific and non-specific nucleic acid amplification.

In certain embodiments, method of detecting a nucleic acid of interest in a sample, comprises preparing primers complementary to the nucleic acid of interest wherein the primers are protected against nuclease activity; performing isothermal amplification to amplify small amounts of either RNA or DNA sequences from the sample to double stranded DNA (dsDNA); and subjecting the amplified nucleic acid of interest to a gene editing agent. In certain embodiments, the gene editing agent comprises: CRISPR-associated endonuclease/Cas or Cpf1, Argonaute family of endonucleases, clustered regularly interspaced short palindromic repeat (CRISPR) nucleases, restriction enzymes, zinc-finger nucleases (ZFNs), transcription activator-like effector nucleases (TALENs), meganucleases, endo- or exo-nucleases, or combinations thereof. In certain embodiments, the sequence-specific nuclease comprises Cas9.

In certain embodiments, the sample is a biological sample or an environmental sample. The biological sample may be a blood, plasma, serum, urine, stool, sputum, mucous, lymph fluid, synovial fluid, bile, ascites, pleural effusion, seroma, saliva, cerebrospinal fluid, aqueous or vitreous humor, or any bodily secretion, a transudate, an exudate (for example, fluid obtained from an abscess or any other site of infection or inflammation), or fluid obtained from a joint (for example, a normal joint or a joint affected by disease, such as rheumatoid arthritis, osteoarthritis, gout or septic arthritis), or a swab of skin or mucosal membrane surface. The environmental sample may be obtained from a food sample, paper surface, a fabric, a metal surface, a wood surface, aplastic surface, a soil sample, a fresh water sample, a waste water sample, a saline water sample, or a combination thereof.

In certain embodiments, the gene editing agent is guided to the nucleic acid sequence of interest by at least one guide RNA (gRNA) wherein the gRNA is complementary to a target nucleic acid sequence of the nucleic acid of interest. In certain embodiments, the guide RNA with sequence complementary to the target nucleic acid sequence of the nucleic acid of interest target of interest is added to the first volume. In certain embodiments, a guide RNA lacking complementarity to a target nucleic acid sequence is added to the second volume. In certain embodiments, each volume is incubated and DNA concentrations are measured. In certain embodiments, an on-target amplification in the isothermal amplification step results in Cas9 cleavage and exonuclease degradation of the on-target product. In certain embodiments, an off-target amplification product is not cleaved or degraded. In certain embodiments, the nucleic acid of interest is present in a sample as measured by a relative reduction in dsDNA concentration in the first volume versus the second volume.

In certain embodiments, a method of detecting exogenous or abnormal nucleic acid sequences in a subject, comprising comprises preparing primers complementary to the nucleic acid of interest wherein the primers are protected against nuclease activity; performing isothermal amplification to amplify small amounts of either RNA or DNA sequences from the sample to double stranded DNA (dsDNA); and subjecting the amplified nucleic acid of interest to a gene editing agent. In certain embodiments, the gene editing agent comprises: CRISPR-associated endonuclease/Cas or Cpf1, Argonaute family of endonucleases, clustered regularly interspaced short palindromic repeat (CRISPR) nucleases, restriction enzymes, zinc-finger nucleases (ZFNs), transcription activator-like effector nucleases (TALENs), meganucleases, endo- or exo-nucleases, or combinations thereof. In certain embodiments, the sequence-specific nuclease comprises Cas9.

In certain embodiments, an exogenous nucleic acid sequence comprises an infectious disease agent. In certain embodiments, an infectious disease agent comprises: a virus, a latent virus, bacteria, a parasite, a fungus, a prion or combinations thereof. In certain embodiments, an abnormal nucleic acid sequence comprises: a mutation, an oncogene, a single nucleotide polymorphism, a sequence encoding a biomarker, a sequence encoding a tumor antigen or combinations thereof. In certain embodiments, the infection is caused by a virus, a bacterium, a fungus, a protozoan, or a parasite.

In certain embodiments, the infection may be a viral infection. The viral infection may be caused by a DNA virus. The DNA virus may be a Myoviridae, Podoviridae, Siphoviridae, Alloherpesviridae, Herpesviridae (including human herpes virus, and Varicella Zoster virus), Malocoherpesviridae, Lipothrixviridae, Rudiviridae, Adenoviridae, Ampullaviridae, Ascoviridae, Asfarviridae (including African swine fevervirus), Baculoviridae, Cicaudaviridae, Clavaviridae, Corticoviridae, Fuselloviridae, Globuloviridae, Guttaviridae, Hytrosaviridae, Iridoviridae, Maseilleviridae, Mimiviridae, Nudiviridae, Nimaviridae, Pandoraviridae, Papillomaviridae, Phycodnaviridae, Plasmaviridae,Polydnaviruses, Polyomaviridae (including Simian virus 40, JC virus, BK virus), Poxviridae (including Cowpox and smallpox), Sphaerolipoviridae, Tectiviridae, Turriviridae, Dinodnavirus, Salterprovirus, Rhizidovirus. The viral infection may be caused by a double-stranded RNA virus, a positive sense RNA virus, a negative sense RNA virus, a retrovirus, or a combination thereof. The viral infection may be caused by a Coronaviridae virus, a Picornaviridae virus, a Caliciviridae virus, a Flaviviridae virus, a Togaviridae virus, a Bornaviridae, a Filoviridae, a Paramyxoviridae, a Pneumoviridae, a Rhabdoviridae, an Arenaviridae, a Bunyaviridae, an Orthomyxoviridae, or a Delta virus. The viral infection may be caused by Coronavirus, SARS, Poliovirus, Rhinovirus, Hepatitis A, Norwalk virus, Yellow fever virus, West Nile virus, Hepatitis C virus, Dengue fever virus, Zika virus, Rubella virus, Ross River virus, Sindbis virus, Chikungunya virus, Borna disease virus, Ebola virus, Marburgvirus, Measles virus, Mumps virus, Nipah virus, Hendra virus, Newcastle disease virus, Human respiratory syncytial virus, Rabies virus, Lassa virus, Hantavirus, Crimean-Congo hemorrhagic fever virus, Influenza, or Hepatitis D virus

In certain embodiments, the infection is a bacterial infection. The bacterium causing the bacterial infection may be Acinetobacter species, Actinobacillus species, Actinomycetes species, an Actinomyces species, Aerococcus species an Aeromonas species, an Anaplasma species, an Alcaligenes species, a Bacillus species, a Bacteroides species, a Bartonella species, a Bifidobacterium species, a Bordetella species, a Borrelia species, a Brucella species, a Burkholderia species, a Campylobacter species, a Capnocytophaga species, a Chlamydia species, a Citrobacter species, a Coxiella species, a Corynbacterium species, a Clostridium species, an Eikenella species, an Enterobacter species, an Escherichia species, an Enterococcus species, an Ehlichia species, an Epidermophyton species, an Erysipelothrix species, a Eubacterium species,a Francisella species, a Fusobacterium species, a Gardnerella species, a Gemella species, a Haemophilus species, a Helicobacter species, a Kingella species, a Klebsiella species, a Lactobacillus species, a Lactococcus species, a Listeria species, a Leptospira species, a Legionella species, a Leptospira species, Leuconostoc species, a Mannheimia species, a Microsporum species, a Micrococcus species, a Moraxella species, a Morganell species, a Mobiluncus species, a Micrococcus species, Mycobacterium species, a Mycoplasm species, a Nocardia species, a Neisseria species, a Pasteurela species, a Pediococcus species, a Peptostreptococcus species, a Pityrosporum species, a Plesiomonas species, a Prevotella species, a Porphyromonas species, a Proteus species, a Providencia species, a Pseudomonas species, a Propionibacteriums species, a Rhodococcus species, a Rickettsia species, a Rhodococcus species, a Serratia species, a Stenotrophomonas species, a Salmonella species, a Serratia species, a Shigella species, a Staphylococcus species, a Streptococcus species, a Spirillum species, a Streptobacillus species, a Treponema species, a Tropheryma species, a Trichophyton species, an Ureaplasma species, a Veillonella species, a Vibrio species, a Yersinia species, a Xanthomonas species, or combinations thereof.

In certain embodiments, the infection is caused by a fungus. Thef ungus may be Aspergillus, Blastomyces, Candidiasis, Coccidiodomycosis, Cryptococcus neoformans, Cryptococcus gatti, sp. Histoplasma sp. (such as Histoplasma capsulatum), Pneumocystis sp. (such as Pneumocystis jirovecii), Stachybotrys (such as Stachybotrys chartarum), Mucroymcosis, Sporothrix, fungal eye infections ringworm, Exserohilum, Cladosporium, Geotrichum, Saccharomyces, a Hansenula species, a Candida species, a Kluyveromyces species, a Debaryomyces species, a Pichia species, a Penicillium species, a Cladosporium species, a Byssochlamys species or a combination thereof.

In certain embodiments, the infection is caused by a protozoan. The protozoan may be Euglenozoa, a Heterolobosea, a Diplomonadida, an Amoebozoa, a Blastocystic, an Apicomplexa, or combination thereof.

In certain embodiments, the infection is caused by a parasite. The parasite may be Trypanosoma cruzi (Chagas disease), T. brucei gambiense, T. brucei rhodesiense, Leishmania braziliensis, L. infantum, L. mexicana,L. major, L. tropica, L. donovani, Naegleria fowleri, Giardiaintestinalis (G. lamblia, G. duodenalis), Canthamoeba castellanii, Balamuthia madrillaris, Entamoeba histolytica, Blastocystic hominis, Babesia micron, Cryptosporidium parvum, Cyclospora cayetanensis, Plasmodium falciparum, P. vivax, P. ovaloe, P. malariae, and Toxoplasmagondii, or combination thereof.

In certain embodiments, a method of treating a subject comprising diagnosing a disease state comprises detecting an exogenous or abnormal nucleic acid sequences in a subject, administering to the subject a therapy for the diagnosed disease state; and, treating the subject for the diagnosed disease state. In certain embodiments, the disease state is an infection, an organ disease, a blood disease, an immune system disease, a cancer, a brain and nervous system disease, an endocrine disease, a pregnancy or childbirth-related disease, an inherited disease, or an environmentally-acquired disease. The disease state may be characterized by the presence or absence of an antibiotic or drug resistance or susceptibility gene or transcript or polypeptide, preferably in a pathogen or a cell. The target molecule may be an antibiotic or drug resistance or susceptibility gene or transcript or polypeptide.

In certain embodiments, the one or more target molecules are diagnostic for a disease state. The disease state may be cancer. The disease state may be an autoimmune disease. The disease state may be an infection, e.g. virus infection.

In certain embodiments, a method of detecting SARS-CoV-2 in a biological sample, comprising preparing primers complementary to a SARS-CoV-2 nucleic acid of interest wherein the primers are protected against nuclease activity; performing isothermal amplification to amplify small amounts of either RNA or DNA sequences from the sample to double stranded DNA (dsDNA); subjecting the amplified SARS-CoV-2 nucleic acid of interest to a gene editing complex comprising at least one guide RNA (gRNA) complementary to a SARS-CoV-2 target nucleic acid sequence of interest and, detecting the SARS-CoV-2 target nucleic acid of interest. In certain embodiments, the at least one gRNA comprises a sequence having at least a 75% sequence identity to the SARS-CoV-2 target nucleic acid sequence of interest. In certain embodiments, a gRNA comprises one or more sequences having at least 75% sequence identity to SEQ ID NOS: 18-24. In certain embodiments, the gRNA comprises one or more sequences comprising SEQ ID NOS: 18-24. In certain embodiments, the primers comprises any one or more of SEQ ID NOS: 1-17.

In certain embodiments, the methods embodied herein are high throughput assays.

Other aspects are described infra.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used. It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element. Thus, recitation of “a cell”, for example, includes a plurality of the cells of the same type. Furthermore, to the extent that the terms “including”, “includes”, “having”, “has”, “with”, or variants thereof are used in either the detailed description and/or the claims, such terms are intended to be inclusive in a manner similar to the term “comprising.”

The terms “amplify”, “amplification”, “amplification reaction”, or “amplifying” refer to any in vitro process for multiplying the copies of a target nucleic acid. Amplification sometimes refers to an “exponential” increase in target nucleic acid. However, “amplifying” may also refer to linear increases in the numbers of a target nucleic acid, but is different than a one-time, single primer extension step. In some embodiments a limited amplification reaction, also known as pre-amplification, can be performed. Pre-amplification is a method in which a limited amount of amplification occurs due to a small number of cycles, for example 10 cycles, being performed. Pre-amplification can allow some amplification, but stops amplification prior to the exponential phase, and typically produces about 500 copies of the desired nucleotide sequence(s). Use of pre-amplification may limit inaccuracies associated with depleted reactants in certain amplification reactions, and also may reduce amplification biases due to nucleotide sequence or species abundance of the target. In some embodiments a one-time primer extension may be performed as a prelude to linear or exponential amplification.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

The term “complementary” refer to oligonucleotides related by base-pairing rules. Complementary nucleotides are, generally, A and T (or A and U), or C and G. For example, for the sequence “5′-AGT-3′,” the perfectly complementary sequence is“3′-TCA-5′.” Methods for calculating the level of complementarity between two nucleic acids are widely known to those of ordinary skill in the art. For example, complementarity may be computed using online resources, such as, e.g., the NCBI BLAST website (ncbi.nlm.nih.gov/blast/producttable.shtml) and the Oligonucleotides Properties Calculator on the Northwestern University website (basic.northwestern.edu/biotools/oligocalc.html). Two single-stranded RNA or DNA molecules may be considered substantially complementary when the nucleotides of one strand, optimally aligned and with appropriate nucleotide insertions or deletions, pair with at least about 80% of the nucleotides of the other strand, usually at least about 90% to 95%, or about 98 to 100%. Two single-stranded oligonucleotides are considered perfectly complementary when the nucleotides of one strand, optimally aligned and with appropriate nucleotide insertions or deletions, pair with 100% of the nucleotides of the other strand. Alternatively, substantial complementarity exists when a first oligonucleotide will hybridize under selective hybridization conditions to a second oligonucleotide. Selective hybridization conditions include, but are not limited to, stringent hybridization conditions. Selective hybridization, or substantially complementary hybridization, occurs when at least about 65% of the nucleic acid sequences within a first oligonucleotide over a stretch of at least 14 to25 sequences pair with a perfectly complementary sequences within a second oligonucleotide, preferably at least about 75%, more preferably at least about 90%. In certain embodiments, the two nucleic acid sequences have at least95%, 96%, 97%, 98%, 99% or 100% of sequence identity. See, M. Kanehisa, Nucleic Acids Res. 12, 203 (1984), incorporated herein by reference. For shorter nucleotide sequences selective hybridization occurs when at least about 65% of the nucleic acid sequences within a first oligonucleotide over a stretch of at least 8 to 12 nucleotides pair with a perfectly complementary nucleic acid sequence within a second oligonucleotide, or at least about 75%, more preferably at least about 90%.Stringent hybridization conditions will typically include salt concentrations of less than about 1 M, more usually less than about 500 mM and preferably less than about 200 mM. Hybridization temperatures can be as low as 5° C., and are preferably lower than about 30° C. However, longer fragments may require higher hybridization temperatures for specific hybridization. Hybridization temperatures are generally at least about 2° C. to 6° C. lower than melting temperatures (Tₘ), which are defined below.

As used herein, the term “crRNA” or “guide RNA” or “single guide RNA,” “gRNA” refers to a polynucleotide comprising any polynucleotide sequence having sufficient complementarity with a target nucleic acid sequence to hybridize with the target nucleic acid sequence and to direct sequence-specific binding of an RNA-targeting complex comprising the gRNA and a CRISPR effector protein to the target nucleic acid sequence. In general, a gRNA may be any polynucleotide sequence (i) being able to form a complex with a CRISPR effector protein and (ii) comprising a sequence having sufficient complementarity with a target polynucleotide sequence to hybridize with the target sequence and direct sequence-specific binding of a CRISPR complex to the target sequence. As used herein the term “capable of forming a complex with the CRISPR effector protein” refers to the gRNA having a structure that allows specific binding by the CRISPR effector protein to the gRNA such that a complex is formed that is capable of binding to a target RNA in a sequence specific manner and that can exert a function on said target RNA. Structural components of the gRNA may include direct repeats and a guide sequence (or spacer). The sequence specific binding to the target RNA is mediated by a part of the gRNA, the “guide sequence”, being complementary to the target RNA. In embodiments of the invention the term guide RNA, i.e. RNA capable of guiding Cas to a target locus, is used as in foregoing cited documents such as WO 2014/093622 (PCT/US2013/074667). As used herein the term “wherein the guide sequence is capable of hybridizing” refers to a subsection of the gRNA having sufficient complementarity to the target sequence to hybridize thereto and to mediate binding of a CRISPR complex to the target RNA. In general, a guide sequence is any polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies; available at novocraft.com), ELAND (Illumina, San Diego, Calif), SOAP (available at soap.genomics.org.cn), and Maq (available at maq.sourceforge.net).

The term “nucleic acid” refers to natural nucleic acids, artificial nucleic acids, analogs thereof, or combinations thereof. The term is inclusive of deoxyribonucleotides, ribonucleotides, and 2′-modified nucleotides) and polymers thereof in either single-, double-or multiple-stranded form, or complements thereof. Nucleic acids may also include analogs of DNA or RNA having modifications to either the bases or the backbone. For example, nucleic acid, includes the use of peptide nucleic acids (PNA). The term “nucleic acids” also includes chimeric molecules. Nucleic acids, including e.g., nucleic acids with a phosphorothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent, or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine.; and peptide nucleic acid backbones and linkages. Other analog nucleic acids include those with positive backbones; non-ionic backbones, modified sugars, and non-ribose backbones (e.g. phosphorodiamidate morpholino oligonucleotides or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Reference throughout this specification to “one embodiment”, “an embodiment,” “in certain embodiments,” means that a particular feature, structure or characteristic described in connection with the embodiment is included in at least one embodiment of the present disclosure. Thus, appearances of the phrases “in one embodiment,” “in an embodiment,” or “ in certain embodiments,” in various places throughout this specification are not necessarily all referring to the same embodiment, but may. Furthermore, the particular features, structures or characteristics maybe combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. In embodiments, the percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “polynucleotide,” “oligonucleotide,” “oligo” or the like refer, in the usual and customary sense, to a linear sequence of nucleotides. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness.

The term “sample” in the present specification and claims is used in its broadest sense and can be, by non-limiting example, any sample that is suspected of containing a target sequence(s) to be detected. It is meant to include specimens or cultures (e.g., microbiological cultures), biological and environmental specimens as well as non-biological specimens. Biological samples may comprise animal-derived materials, including fluid (e.g., blood, saliva, urine, lymph, etc.), solid (e.g., stool) or tissue (e.g., buccal, organ-specific, skin, etc.), as well as liquid and solid food and feed products and ingredients such as dairy items, vegetables, meat and meat by-products, and waste. Biological samples may be obtained from, e.g., humans, any domestic or wild animals, plants, bacteria or other microorganisms, etc. Environmental samples can include environmental material such as surface matter, soil, water (e.g., contaminated water), air and industrial samples, as well as samples obtained from food and dairy processing instruments, apparatus, equipment, utensils, disposable and non-disposable items. These examples are not to be construed as limiting the sample types applicable to the present disclosure. Those of skill in the art would appreciate and understand the particular type of sample required for the detection of particular target sequences (Pawliszyn, J., Sampling and Sample Preparation for Field and Laboratory, (2002). Venkatesh Iyengar. G., et al., Element Analysis of Biological Samples: Principles and Practices (1998). Drielak. S., Hot Zone Forensics: Chemical, Biological. and Radiological Evidence Collection (2004); and Nielsen. D. M., Practical Handbook of Environmental Site Characterization and Ground-Water Monitoring (2005)).

The term “target nucleic acid” refers to a nucleic acid (often derived from a biological sample), to which the oligonucleotide is designed to specifically hybridize. It is either the presence or absence of the target nucleic acid that is to be detected, or the amount of the target nucleic acid that is to be quantified. The target nucleic acid has a sequence that is complementary to the nucleic acid sequence of the corresponding oligonucleotide directed to the target. The term target nucleic acid may refer to the specific subsequence of a larger nucleic acid to which the oligonucleotide is directed or to the overall sequence (e.g., gene or mRNA) it is desired to detect. The difference in usage will be apparent from context.

As used herein, the term “Thermal Melting Point (Tₘ)” refers to the temperature, under defined ionic strength, pH, and nucleic acid concentration, at which 50% of the oligonucleotides complementary to the target sequence hybridize to the target sequence at equilibrium. Typically, stringent conditions will be those in which the salt concentration is at least about 0.01 to 1.0 M Na ion concentration (or other salts) at pH 7.0 to 8.3 and the temperature is at least about 30° C. for short oligonucleotides (e.g., 10 to 50 nucleotide). Stringent conditions may also be achieved with the addition of destabilizing agents such as formamide.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 25 41, 42, 43, 44, 45, 46, 47, 48, 49, or 50. Concentrations, amounts, cell counts, percentages and other numerical values may be presented herein in a range format. It is to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

Any compositions or methods provided herein can be combined with one or more of any of the other compositions and methods provided herein.

All publications, published patent documents, and patent applications cited herein are hereby incorporated by reference to the same extent as though each individual publication, published patent document, or patent application was specifically and individually indicated as being incorporated by reference.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a schematic representation showing the steps of the isothermal amplification of SARS-CoV2. Overview of reaction: RT-LAMP amplifies target RNA/DNA, followed by incubation with master mix containing Cas9 with either target or non-targeting guide RNA, lambda exonuclease, and RepX. The readout is dsDNA concentration measured using the Picogreen dye. FIG. 1B is a gel of the isothermal amplification reaction. Use of on-target guide RNA (19n) successfully degrades target DNA, whereas off-target guide RNA (bat) fails to degrade target DNA.

FIG. 2 is a schematic representation of the automated, high-throughput SENTINEL assay on 96-well plates. In this embodiment, the assay utilizes: two plate transfer steps (changes in well color correspond to different plates); five reagent additions (QuickExtract, RT-LAMP master mix, water, SENTINEL master mix, Picogreen buffer) and a 1 hour assay time from patient sample input to readout. 92 patient samples +2 positive controls and 2 negative controls per 96-well plate were tested. A change in reaction volume allows compatibility with 384-well plates, etc.

FIG. 3 is a schematic representation of an embodiment of the assays described herein using CRISPR/Cas as the nuclease. The schematic representation shows a conceptual overview of the mechanism of action. Cas9 induces on-target cleavage, RepX induces eviction of Cas9, and lambda exonuclease loads on the exposed 5′ phosphorylated dsDNA for efficient degradation.

FIG. 4 is a series of graphs demonstrating detection of target nucleic acids at the attomolar level. The limit of detection testing was conducted using either in-vitro transcribed ssRNA or plasmid dsDNA of the SARS-CoV-2 on-target sequence.

FIG. 5 is a graph demonstrating detection of mismatched target nucleic acid sequences (freeze-thaw effect). Use of either human SARS-CoV-2 or bat (bat-SL-CoVZC45) guide RNA or RT-LAMP product were tested. Multiple freeze-thaw cycles of the SENTINEL master mix did not lead to appreciably reduced activity.

FIG. 6 is a graph demonstrating detection of mismatched target nucleic acid sequences by HiFi Cas9 as compared to wild type SpCas9. Use of either human SARS-CoV-2 or bat (bat-SL-CoVZC45) guide RNA or RT-LAMP product was tested. Green and red bars correspond to expected positive or negative tests, respectively. An enhanced specificity Cas9 (HiFi Cas9) is compared to wild type Cas9, exhibiting better performance.

FIG. 7 is a graph demonstrating detection of mismatched target nucleic acid sequences by eSpCas9. Use of either human SARS-CoV-2 or bat (bat-SL-CoVZC45) guide RNA or RT-LAMP product was tested. Green and red bars correspond to expected positive or negative tests, respectively. An enhanced specificity Cas9 (eSpCas9) was demonstrated.

FIG. 8 is a schematic representation of an embodiment of the assays described herein using restriction enzymes. Modification of the protocol for use of a restriction endonuclease (RE) instead of Cas9 is shown. RepX is omitted because RE's depart dsDNA after cleavage, thus exposing 5′ phosphorylated dsDNA ends for lambda exonuclease cleavage

FIG. 9 is a series of graphs demonstrating detection of a single mismatched target nucleic acid sequence by use of the restriction endonuclease. ‘19n-wt’ and ‘19n’ both correspond to RT-LAMP products of on-target human SARS-CoV-2 RNA. ‘19n-prox’ exhibits one mismatch in the RE binding sequence. Green and red bars correspond to expected positive or negative tests, respectively.

FIG. 10 is a graph demonstrating detection of mismatched target nucleic acid sequences by AsCpf1. The use of either human SARS-CoV-2 or bat (bat-SL-CoVZC45) guide RNA or RT-LAMP product was tested. Green and red bars correspond to expected positive or negative tests, respectively. The performance of AsCpf1 in the place of Cas9 is demonstrated.

FIGS. 11A-11L is a series of schematics, graphs and blots demonstrating the characterization of endonuclease actuated nucleic acid detection.

FIG. 11A) Schematic of SENTINEL mechanism of action. Blue circles at the 5′ ends of the RT-LAMP product represent 5′ phosphorothiolate modifications. Red circles at the 5′ ends represent 5′ phosphates exposed from endonuclease action.

FIG. 11B) Cas9 cleavage of SARS-CoV-2 RT-LAMP product using on-target (19n) and non-target (neg) gRNA. On-target gRNA led to downward shift in gel bands, consistent with cleavage of RT-LAMP product.

FIG. 11C) Addition of λ-exo did not lead to appreciable change compared to FIG. 11B.

FIG. 11D) Addition of λ-exo with Rep-X led to significant degradation of RT-LAMP product, only for the sample with on-target gRNA. In contrast, the sample with off-target gRNA was consistent across FIGS. 11B-11D.

FIG. 11E) Quantification of FIGS. 11B-11D. across two biological replicates, by measuring fractional intensities of each lane on the agarose gel relative to the maximum intensity across all lanes. From left to right, the first two lanes quantify FIG. 11B, second two lanes quantify FIG. 11C, and last two lanes quantify FIG. 11D.

FIG. 11F) Schematic of SENTINEL protocol, starting from synthetic, in vitro transcribed viral RNA.

FIGS. 11G-11H) Limit of detection with the SENTINEL assay, with (FIG. 11G) ssRNA and (FIG. 11H) dsDNA input.

FIG. 11I) Result of isothermal amplification (RT-LAMP) alone for nucleic acid detection. Agarose gel of RT-LAMP product with use of either SARS-CoV-2 (19n) or bat-SL-CoVZC45 (bat) RT-LAMP primers for detection of 19n ssRNA or bat ssRNA, respectively. Notably, detection for SARS-CoV-2 (using 19n RT-LAMP primers) resulted in a false positive when bat ssRNA was in the sample.

FIG. 11J) Stability of SENTINEL master mix to freeze-thaw cycles, evaluating for SARS-CoV-2 using its corresponding Cas9/gRNA, on RT-LAMP products of samples containing SARS-CoV-2 (19n, red) or bat-SL-CoVZC45 (bat, grey) ssRNA.

FIG. 11K) Stability of SENTINEL master mix to 1-month storage in −80° C., −20° C., 4° C., or 23° C. (room temperature). The master mix only loses activity after 1-month storage in room temperature. The pairing of gRNA with ssRNA target samples is the same as FIG. 11J.

FIG. 11L) SENTINEL score as a function of room temperature reaction time. The pairing of gRNA with ssRNA target samples is the same as FIGS. 11J and 11K.

FIGS. 11J-11L) All error bars represent ±1 standard deviation from mean, from 3 experimental replicates.

FIGS. 12A-12G are a series of graphs and schematics demonstrating compatibility with different sequence-specific endonucleases and with viral particles in human saliva.

FIGS. 12A-12D) Use of SENTINEL to detect the N-gene of SARS-CoV-2 (19n) vs bat-SL-CoVZC45 (bat), using guide RNA targeting the N-gene of SARS-CoV-2 (19n gRNA) vs bat-SL-CoVZC45 (bat gRNA). (FIG. 12A) wild type SpCas9, (FIG. 12B) HiFi Cas9,

(FIG. 12C) eSpCas9, and (FIG. 12D) AsCpf1 were used as the endonuclease. Light blue bars indicate expected positive samples, while grey bars indicate expected negative samples.

FIG. 12E) Use of Afel as the endonuclease to discriminate between a single-nucleotide difference in sequence between the N-gene of SARS-CoV-2 (19n) and bat-SL-CoVZC45 (bat). CutSmart, and the λ-exo buffer used with other endonucleases, were both compatible.

FIGS. 12A-12E) All error bars represent +/−1 standard deviation from mean, from 3 experimental replicates plotted as round data points. n. s. indicates not significant, * indicates p<0.05, ** indicates p<0.01, and *** indicates p<0.001 from Student's t-test.

FIG. 12F) Schematic of SARS-CoV-2 viral particle detection from human saliva using SENTINEL.

FIG. 12G) Limit of detection from serial dilutions of viral particles in viral transport media, which was mixed into saliva before subject to the SENTINEL assay.

FIGS. 13A and 13B are a schematic representation showing the template and guide RNA sequences. Sections of FIG. 13A) SARS-CoV-2 and FIG. 13B) bat-SL-CoVZC45 N-gene sequences, amplified by LAMP primers, that contain endonuclease targeting sites. The two Cas9 target sequences (Cas9-19n-N2-gRNA#1, Cas9-19n-N2-gRNA#2) and one AsCpfl target sequence (Cpf1-19n-N2-gRNA#1) are shown.

FIGS. 14A-14C are a series of graphs and an equation demonstrating the analysis and interpretation of SENTINEL scores.

FIG. 14A) Formula for SENTINEL score (top row). Calculation of SENTINEL scores for samples with the expected target DNA sequence (+) or without the expected DNA sequence (−). Note that the difference in scores (6.74 vs 1.16) is mainly driven by the difference in A (given B and C are similar).

FIG. 14B) Perturbation analysis by varying the variable C, keeping other variables the same as FIG. 14A. The vertical red dotted line corresponds to the values from FIG. 14A.

FIG. 14C) Perturbation analysis by varying the variable A relative to B (A/B), keeping other variables the same as FIG. 14A.

FIGS. 15A-15G are a series of graphs showing SENTINEL scores and their corresponding percent reductions in relative DNA concentrations compared to non-target control, measured by fluorescence.

FIG. 15A) Stability of SENTINEL master mix to freeze-thaw cycles, evaluating for bat-SL-CoVZC45 using its corresponding Cas9/gRNA, on RT-LAMP products of samples containing bat-SL-CoVZC45 (bat, red) or SARS-CoV2 (19n, grey) ssRNA.

FIGS. 15B-15C) Stability of SENTINEL master mix to freeze-thaw cycles, evaluating (FIG. 15B) SARS-CoV-2 (19n) vs (FIG. 15C) bat-SL-CoVZC45 (bat) gRNA. The corresponding percent reductions in RT-LAMP product from on-target vs non-target gRNA are shown.

FIG. 15D) Stability of SENTINEL master mix to 1-month storage in −80° C., −20° C., 4° C., or 23° C. (room temperature). The master mix only loses activity after 1-month storage at 15 room temperature. The pairing of gRNA with ssRNA target samples is the same as FIG. 15A.

FIG. 15E) SENTINEL score as a function of room temperature reaction time. The pairing of gRNA with ssRNA target samples is the same as FIGS. 15A and 15D.

FIGS. 15F-15G) SENTINEL score as a function of room temperature reaction time, evaluating (FIG. 15F) SARS-CoV-2 (19n) vs (FIG. 15G) bat-SL-CoVZC45 (bat) gRNA. The corresponding percent reductions in RT-LAMP product from on-target vs non-target gRNA are shown.

FIGS. 15A-15G) All error bars represent ±1 standard deviation from mean, from 3 experimental replicates.

### DETAILED DESCRIPTION

The disclosure is based on the finding of novel assays in the detection and identification of target nucleic acids in a sample. The assays includes the steps of target nucleic acid amplification and subjecting the amplified target nucleic acids to sequence specific cleavage. Briefly, the method utilizes two steps to determine the presence of a nucleic acid of interest. First, isothermal amplification methods, such as, for example, loop-mediated isothermal amplification (LAMP), Recombinase polymerase amplification (RPA), nucleic acid sequence-based amplification (NASBA), strand displacement amplification (SDA), helicase-dependent amplification (HDA), nicking enzyme amplification reaction (NEAR), PCR, multiple displacement amplification (MDA), rolling circle amplification (RCA), ligase chain reaction (LCR), ramification amplification method (RAM) or Whole Genome Amplification (WGA). This step amplifies small amounts of either RNA or DNA from the sample to double stranded DNA (dsDNA). Inhibitors of nucleases or phosphorothiolate bonds are used to cap the ends of primers to appropriately protect the dsDNA products from exonuclease activity in the next step. Isothermal amplification methods alone are also used for nucleic acid detection, but can be non-specific due to unintended amplification of similar sequences. The next step utilizes sequence specific nuclease such as, for example, CRISPR enzyme guided to the target sequence(s) by one or more guide RNAs (gRNAs), followed by its nuclease activity as an actuator to allow convenient discrimination between specific and non-specific amplification.

Accordingly, in certain embodiments a method of detecting a nucleic acid of interest in a sample, comprises preparing primers complementary to the nucleic acid of interest wherein the primers are protected against nuclease activity; performing isothermal amplification to amplify small amounts of either RNA or DNA sequences from the sample to double stranded DNA (dsDNA); subjecting the amplified nucleic acid of interest to a sequence-specific nuclease; and, detecting the nucleic acid of interest.

In certain embodiments, method of detecting a nucleic acid of interest in a sample, comprises preparing primers complementary to the nucleic acid of interest wherein the primers are protected against nuclease activity; performing isothermal amplification to amplify small amounts of either RNA or DNA sequences from the sample to double stranded DNA (dsDNA); and subjecting the amplified nucleic acid of interest to a gene editing agent. In certain embodiments, the gene editing agent comprises: CRISPR-associated endonuclease/Cas or Cpf1, Argonaute family of endonucleases, clustered regularly interspaced short palindromic repeat (CRISPR) nucleases, restriction enzymes, zinc-finger nucleases (ZFNs), transcription activator-like effector nucleases (TALENs), meganucleases, endo- or exo-nucleases, or combinations thereof. In certain embodiments, the sequence-specific nuclease comprises Cas9.

In certain embodiments, a method of detecting exogenous or abnormal nucleic acid sequences in a subject, comprising comprises preparing primers complementary to the nucleic acid of interest wherein the primers are protected against nuclease activity; performing isothermal amplification to amplify small amounts of either RNA or DNA sequences from the sample to double stranded DNA (dsDNA); and subjecting the amplified nucleic acid of interest to a gene editing agent. In certain embodiments, the gene editing agent comprises: CRISPR-associated endonuclease/Cas or Cpf1, Argonaute family of endonucleases, clustered regularly interspaced short palindromic repeat (CRISPR) nucleases, restriction enzymes, zinc-finger nucleases (ZFNs), transcription activator-like effector nucleases (TALENs), meganucleases, endo- or exo-nucleases, or combinations thereof. In certain embodiments, the sequence-specific nuclease comprises Cas9. In certain embodiments, an exogenous nucleic acid sequence comprises an infectious disease agent. In certain embodiments, an infectious disease agent comprises: a virus, a latent virus, bacteria, a parasite, a fungus, a prion or combinations thereof. In certain embodiments, an abnormal nucleic acid sequence comprises: a mutation, an oncogene, a single nucleotide polymorphism, a sequence encoding a biomarker, a sequence encoding a tumor antigen or combinations thereof.

### Target Sequences

In certain embodiments, one or more nucleic acid targets are amplified. Target nucleic acids may be referred to as target sequences, target polynucleotides, and/or target polynucleotide sequences, and may include double-stranded and single-stranded nucleic acid molecules. Target nucleic acid may be, for example, DNA or RNA. Where the target nucleic acid is an RNA molecule, the molecule may be, for example, double-stranded, single-stranded, or the RNA molecule may comprise a target sequence that is single-stranded. Where the target nucleic acid is double stranded, the target nucleic acid generally includes a first strand and a second strand. A first strand and a second strand may be referred to as a forward strand and a reverse strand and generally are complementary to each other. Where the target nucleic acid is single stranded, a complementary strand may be generated, for example by polymerization and/or reverse transcription, rendering the target nucleic acid double stranded and having a first/forward strand and a second/reverse strand.

A target sequence may refer to either the sense or antisense strand of a nucleic acid sequence, and also may refer to sequences as they exist on target nucleic acids, amplified copies, or amplification products, of the original target sequence. A target sequence may be a subsequence within a larger polynucleotide. For example, a target sequence may be a short sequence (e.g., 20 to 50 bases) within a nucleic acid fragment, a chromosome, a plasmid, that is targeted for amplification. In some embodiments, a target sequence may refer to a sequence in a target nucleic acid that is complementary to an oligonucleotide (e.g., primer) used for amplifying a nucleic acid. Thus, a target sequence may refer to the entire sequence targeted for amplification or may refer to a subsequence in the target nucleic acid where an oligonucleotide binds. An amplification product may be a larger molecule that comprises the target sequence, as well as at least one other sequence, or other nucleotides. In some embodiments, an amplification product is about the same length as the target sequence. In some embodiments, an amplification product is exactly the same length as the target sequence. In some embodiments, an amplification product comprises the target sequence. In some embodiments, an amplification product consists of the target sequence.

Target nucleic acid may include, for example, genomic nucleic acid, plasmid nucleic acid, mitochondrial nucleic acid, cellular nucleic acid, extracellular nucleic acid, bacterial nucleic acid, viral nucleic acid and parasite nucleic acid. In some embodiments, target nucleic acid may include genomic DNA, chromosomal DNA, plasmid DNA, mitochondrial DNA, a gene, any type of cellular RNA, messenger RNA, bacterial RNA, viral RNA or a synthetic oligonucleotide. Genomic nucleic acid may include any nucleic acid from any genome, for example, including animal, plant, insect, parasitic, viral and bacterial genomes, including, for example, genomes present in spores. In some embodiments, genomic target nucleic acid may be within a particular genomic locus or a plurality of genomic loci. A genomic locus may include any or a combination of open reading frame DNA, non-transcribed DNA, non-coding sequences, intron sequences, exon sequences, promoter sequences, enhancer sequences, flanking sequences, or any sequences considered associated with a given genomic locus.

In some embodiments, a target sequence comprises one or more repetitive elements (e.g., multiple repeat sequences, inverted repeat sequences, palindromic sequences, tandem repeats, microsatellites, minisatellites, and the like). In some embodiments, a target sequence is present within a sample nucleic acid (e.g., within a nucleic acid fragment, a chromosome, a genome, a plasmid) as a repetitive element(e.g., a multiple repeat sequence, an inverted repeat sequence, a palindromic sequence, a tandem repeat, a microsatellite repeat, a minisatellite repeat and the like). For example, a target sequence may occur multiple times as a repetitive element and one, some, or all occurrences of the target sequence within a repetitive element may be amplified (e.g., using a single pair of primers) using methods described herein. In some embodiments, a target sequence is present within a sample nucleic acid (e.g., within a nucleic acid fragment, a chromosome, a genome, a plasmid) as a duplication and/or a paralog.

Target nucleic acid sequences may include microRNAs. MicroRNAs, miRNAs, or small temporal RNAs (stRNAs) are short (e.g., about 21 to 23 nucleotides long) and single-stranded RNA sequences involved in gene regulation. MicroRNAs may interfere with translation of messenger RNAs and are partially complementary to messenger RNAs. Target nucleic acid may include microRNA precursors such as primary transcript (pri-miRNA) and pre-miRNA stem-loop-structured RNA that is further processed into miRNA. Target nucleic acid may include short interfering RNAs (siRNAs), which are short (e.g., about 20 to 25 nucleotides long) and at least partially double-stranded RNA molecules involved in RNA interference (e.g., down-regulation of viral replication or gene expression).

Nucleic acid utilized in methods described herein may be obtained from any suitable biological specimen or sample, and often is isolated from a sample obtained from a subject. A subject can be any living or non-living organism, including but not limited to a human, a non-human animal, a plant, a bacterium, a fungus, a virus, or a protist. Any human or non-human animal can be selected, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. A subject may be a male or female, and a subject may be any age (e.g., an embryo, a fetus, infant, child, adult).

A sample or test sample can be any specimen that is isolated or obtained from a subject or part thereof. Non-limiting examples of specimens include fluid or tissue from a subject, including, without limitation, blood or a blood product (e.g., serum, plasma, or the like), umbilical cord blood, bone marrow, chorionic villi, amniotic fluid, cerebrospinal fluid, spinal fluid, lavage fluid (e.g., bronchoalveolar, gastric, peritoneal, ductal, ear, arthroscopic), biopsy sample, celocentesis sample, cells (e.g., blood cells) or parts thereof (e.g., mitochondrial, nucleus, extracts, or the like), washings of female reproductive tract, urine, feces, sputum, saliva, nasal mucous, prostate fluid, lavage, semen, lymphatic fluid, bile, tears, sweat, breast milk, breast fluid, hard tissues (e.g., liver, spleen, kidney, lung, or ovary),the like or combinations thereof. The term blood encompasses whole blood, blood product or any fraction of blood, such as serum, plasma, buffy coat, or the like as conventionally defined. Blood plasma refers to the fraction of whole blood resulting from centrifugation of blood treated with anticoagulants. Blood serum refers to the watery portion of fluid remaining after a blood sample has coagulated. Fluid or tissue sample soften are collected in accordance with standard protocols hospitals or clinics generally follow. For blood, an appropriate amount of peripheral blood (e.g., between 3-40 milliliters) often is collected and can be stored according to standard procedures prior to or after preparation.

A sample or test sample can include samples containing spores, viruses, cells, nucleic acid from prokaryotes or eukaryotes, or any free nucleic acid. For example, a method described herein may be used for detecting nucleic acid on the outside of spores (e.g., without the need for lysis). A sample may be isolated from any material suspected of containing a target sequence, such as from a subject described above. In certain instances, a target sequence may be present in air, plant, soil, or other materials suspected of containing biological organisms.

Nucleic acid may be derived (e.g., isolated, extracted, purified) from one or more sources by methods known in the art. Any suitable method can be used for isolating, extracting and/or purifying nucleic acid from a biological sample, non-limiting examples of which include methods of DNA preparation in the art, and various commercially available reagents or kits, such as Qiagen's QIAamp Circulating Nucleic Acid Kit, QiaAmp DNAMini Kit or QiaAmp DNA Blood Mini Kit (Qiagen, Hilden, Germany), GENOMICPREPT™, Blood DNA Isolation Kit (Promega, Madison, WI.), GFX™ Genomic Blood DNA Purification Kit (Amersham, Piscataway, N.J.), and the like or combinations thereof.

In some embodiments, a cell lysis procedure is performed. Cell lysis may be performed prior to initiation of an amplification reaction described herein (e.g., to release DNA and/or RNA from cells for amplification). Cell lysis procedures and reagents are known in the art and may generally be performed by chemical (e.g., detergent, hypotonic solutions, enzymatic procedures, and the like, or combination thereof), physical (e.g., French press, sonication, and the like), or electrolytic lysis methods. Any suitable lysis procedure can be utilized. For example, chemical methods generally employ lysing agents to disrupt cells and extract nucleic acids from the cells, followed by treatment with chaotropic salts. In some embodiments, cell lysis comprises use of detergents (e.g., ionic, nonionic, anionic, zwitterionic). In some embodiments, cell lysis comprises use of ionic detergents (e.g., sodium dodecyl sulfate (SDS), sodium lauryl sulfate (SLS), deoxycholate, cholate, sarkosyl) Physical methods such as freeze/thaw followed by grinding, the use of cell presses and the like also may be useful. High salt lysis procedures also may be used. For example, an alkaline lysis procedure may be utilized. The latter procedure traditionally incorporates the use of phenol-chloroform solutions, and an alternative phenol-chloroform-free procedure involving three solutions may be utilized. In the latter procedures, one solution can contain 15 mM Tris, pH 8.0; 10 mM EDTA and 100 μg/ml RNAse A; a second solution can contain 0.2N NaOH and 1% SDS; and a third solution can contain 3M KOAc, pH 5.5, for example. In some embodiments, a cell lysis buffer is used in conjunction with the methods and components described herein.

Nucleic acid may be provided for conducting the methods embodied herein without processing of the sample(s) containing the nucleic acid. For example, in some embodiments, nucleic acid is provided for conducting amplification methods described herein without prior nucleic acid purification. In some embodiments, a target sequence is amplified directly from a sample (e.g., without performing any nucleic acid extraction, isolation, purification and/or partial purification steps). In some embodiments, nucleic acid is provided for conducting methods described herein after processing of the sample(s) containing the nucleic acid. For example, a nucleic acid can be extracted, isolated, purified, or partially purified from the sample(s). The term “isolated” generally refers to nucleic acid removed from its original environment(e.g., the natural environment if it is naturally occurring, or a host cell if expressed exogenously), and thus is altered by human intervention (e.g., “by the hand of man”) from its original environment. The term “isolated nucleic acid” can refer to a nucleic acid removed from a subject (e.g., a human subject). An isolated nucleic acid can be provided with fewer non-nucleic acid components (e.g., protein, lipid, carbohydrate) than the amount of components present in a source sample. A composition comprising isolated nucleic acid can be about 50% to greater than 99% free of non-nucleic acid components. A composition comprising isolated nucleic acid can be about 90%, 91%, 92%, 93%, 94%, 95%, 96%,97%, 98%, 99% or greater than 99% free of non-nucleic acid components. The term “purified” generally refers to a nucleic acid provided that contains fewer non-nucleic acid components (e.g., protein, lipid, carbohydrate) than the amount of non-nucleic acid components present prior to subjecting the nucleic acid to a purification procedure. A composition comprising purified nucleic acid may be about 80%, 81%, 82%,83%, 84%, 85%, 86%, 87%, 88%, 89%, 90%, 91%, 92%, 93%, 94%, 95%, 96%,97%, 98%, 99% or greater than 99% free of other non-nucleic acid components.

Nucleic acid may be provided for conducting methods described herein without modifying the nucleic acid. Modifications may include, for example, denaturation, digestion, nicking, unwinding, incorporation and/or ligation of heterogeneous sequences, addition of epigenetic modifications, addition of labels (e.g., radiolabels such as ³²P, ³³P, ¹²⁵I, or ³⁵S; enzyme labels such as alkaline phosphatase; fluorescent labels such as fluorescein isothiocyanate(FITC); or other labels such as biotin, avidin, digoxigenin, antigens, haptens, fluorochromes), and the like. Accordingly, in some embodiments, an unmodified nucleic acid is amplified.

### Amplification

In certain embodiments, amplification of nucleic acid comprises a non-thermocycling type of polymerase chain reaction (PCR). In certain embodiments, amplification of nucleic acid comprises an isothermal amplification process. In some embodiments, amplification of nucleic acid comprises an isothermal polymerase chain reaction (iPCR). Isothermal amplification generally is an amplification process performed at a constant temperature. Terms such as isothermal conditions, isothermally and constant temperature generally refer to reaction conditions where the temperature of the reaction is kept essentially constant during the course of the amplification reaction. Isothermal amplification conditions generally do not include a thermocycling (i.e., cycling between an upper temperature and a lower temperature) component in the amplification process. When amplifying under isothermal conditions, the reaction may be kept at an essentially constant temperature, which means the temperature may not be maintained at precisely one temperature. For example, small fluctuations in temperature (e.g., ±1° C. to 5° C. may occur in an isothermal amplification process due to, for example, environmental or equipment-based variables. Often, the entire reaction volume is kept at an essentially constant temperature, and isothermal reactions herein generally do not include amplification conditions that rely on a temperature gradient generated within a reaction vessel and/or convective-flow based temperature cycling.

Isothermal amplification reactions may be conducted at an essentially constant temperature. In some embodiments, isothermal amplification reactions herein are conducted at a temperature of about 55° C. to a temperature of about 75° C. For example, isothermal amplification reactions may be conducted at a temperature of about 55° C., about 56° C., about 57° C., about 58° C., about 59° C., about 60° C., about 61° C., about 62° C., about 63° C., about 64° C., about 65° C., about 66° C., about 67° C. ,about 68° C., about 69° C. s, about 70° C., about 71° C., about 72° C., about 73° C., about 74° C., or about 75° C. In some embodiments, isothermal amplification reactions are conducted at a temperature of about 55° C. to a temperature of about 65° C. For example, isothermal amplification reactions may be conducted at a temperature of about 60° C. Isothermal amplification reactions herein may be conducted at a temperature of about 65° C. In some embodiments, a temperature element (e.g., heat source) is kept at an essentially constant temperature. In some embodiments, a temperature element is kept at an essentially constant temperature at or below about 75° C. In some embodiments, a temperature element is kept at an essentially constant temperature at or below about 70° C. In some embodiments, a temperature element is kept at an essentially constant temperature at or below about 65° C. In some embodiments, a temperature element is kept at an essentially constant temperature at or below about 60° C.

An amplification process herein may be conducted over a certain length of time. In some embodiments, an amplification process is conducted until a detectable nucleic acid amplification product is generated. A nucleic acid amplification product may be detected by any suitable detection process and/or a detection process described herein. In some embodiments, an amplification process is conducted over a length of time within about 20 minutes or less. For example, an amplification process may be conducted within about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, or about 20 minutes. In some embodiments, an amplification process is conducted over a length of time within about 10 minutes or less.

In embodiments, the target RNAs and/or DNAs sequences in a sample are amplified prior to subjecting the amplified products to a sequence specific nuclease. Any suitable RNA or DNA amplification technique may be used. In certain embodiments, the RNA or DNA amplification is an isothermal amplification. In certain embodiments, the isothermal amplification comprises nucleic-acid sequence-based amplification (NASBA), recombinase polymerase amplification (RPA), loop-mediated isothermal amplification (LAMP), real-time loop-mediated isothermal amplification (RT-LAMP), strand displacement amplification (SDA), helicase-dependent amplification (HDA), or nicking enzyme amplification reaction (NEAR). In certain embodiments, non-isothermal amplification methods may be used which include, but are not limited to, PCR, multiple displacement amplification (MDA), rolling circle amplification (RCA), ligase chain reaction (LCR), ramification amplification method (RAM) cross-priming amplification (CPA) or smart amplification (SMAP).

Loop-mediated isothermal amplification (LAMP) is a rapid and sensitive gene amplification method. LAMP reactions are conducted at a single temperature (usually between 60-65° C.), and results are usually obtained within one hour. In the past few years, LAMP has been integrated into lab-on-a-chip technologies. Accordingly, in certain embodiments, the RNA or DNA amplification nucleic acid sequence-based amplification is a loop-mediated isothermal amplification (LAMP) reaction. LAMP (Notomi et al., 2000, Loop-mediated isothermal amplification of DNA. Nucleic Acids Res. 28(12):E63) requires two inner primers and two outer primers. The inner primers are called the forward inner primer(FIP) and the backward inner primer (BIP) and each contains two target-specific portions corresponding to the sense and antisense sequences of the target DNA, one for priming the first stage and the other for self-priming in later stages. The extension of the outer primers in the first stages causes the extension product of the respective inner primer to be displaced by the strand displacement polymerase. In subsequent stages, the forward primers can hybridize to a single-stranded loop structure which is formed by the hybridization of the 5′ upstream target-specific portion to its complement in the extension product. Further primers may be included to accelerate the reaction (Nagamine et al., 2002, Accelerated reaction by loop-mediated isothermal amplification using loop primers. Mol Cell Probes. 16(3):223-9). In an embodiment, the FIP and/or BIP primer may be modified to include the fluorescence cassette-specific portion between the 5′ upstream and 3′ downstream target-specific portions. In “single” and “double” cross priming amplification (CPA) (Xu et al., 2012, Cross priming amplification: mechanism and optimization for isothermal DNA amplification. Sci Rep. 2:246), either or both ends of the amplification product respectively include a loop formed by a cross primer. In “single” CPA, the cross primer contains 5′ tail sequences which is identical to a primer for the complementary strand. It allows for strand displacement by extension from an upstream primer, and incorporates a second defined priming site in the 5′ end of the resulting product. Primers for the opposite strand each consist of a single sequence complementary to the template, and are chosen so that they bind in tandem to the template, providing a region of nicked double stranded DNA which can be extended by a strand displacing DNA polymerase. In a “double” CPA, forward and reverse antisense primers are used, and each cross primer contains 5′ tail sequences identical to each other's priming site, thereby introducing additional priming sites in each round of extension. In an embodiment, the cross primer in “single” CPA, or one or both cross primers in “double” CPA may be modified to include the fluorescence cassette-specific portion between the 5′ upstream and 3′downstream target-specific portions.

In smart amplification (SMAP) (Lezhava and Hayashizaki, 2009. Detection of SNP by the isothermal smart amplification method, and Cross-Priming Amplification (CPA) Methods Mol Biol. 578:437-51), a tailed turn-back primer (TP) and a tailed folding primer(FP) prime extension reactions from either strand, and are themselves displaced by outer primers. A boost primer is typically also included .The outer primers and the TP may respectively be the same as the outer primers and FIP or BIP used in LAMP. However, the FP differs from FIP or BIP in that its tail is not a target-specific portion, but is a self-annealing portion, which can exist as a hairpin. This facilitates self-primed DNA synthesis from the FP, thus perpetuating the reaction. In particular, when a complement to the folding section is generated, the 3′-end of the synthesis product self-hybridizes and acts as a primer to synthesize an additional strand of the target. In an embodiment, the TP may be modified to include the fluorescence cassette-specific portion between the 5′ upstream and 3′ downstream target-specific portions.

In nucleic-acid sequence-based amplification (NASBA), amplification is initiated with reverse transcription of target RNA by a sequence-specific reverse primer to create an RNA/DNA duplex. RNase H is then used to degrade the RNA template, allowing a forward primer containing a promoter, such as the T7 promoter, to bind and initiate elongation of the complementary strand, generating a double-stranded DNA product. The RNA polymerase promoter-mediated transcription of the DNA template then creates copies of the target RNA sequence. The NASBA reaction has the additional advantage of being able to proceed under moderate isothermal conditions, for example at approximately 41° C., making it suitable for systems and devices deployed for early and direct detection in the field and far from clinical laboratories.

Recombinase polymerase amplification (RPA) reactions employ recombinases which are capable of pairing sequence-specific primers with homologous sequences in duplex DNA. If target DNA is present, DNA amplification is initiated and no other sample manipulation such as thermal cycling or chemical melting is required. The entire RPA amplification system is stable as a dried formulation and can be transported safely without refrigeration. RPA reactions may also be carried out at isothermal temperatures with an optimum reaction temperature of 37-42° C. The sequence specific primers are designed to amplify a sequence comprising the target nucleic acid sequence to be detected. In certain example embodiments, an RNA polymerase promoter, such as a T7 promoter, is added to one of the primers. This results in an amplified double-stranded DNA product comprising the target sequence and an RNA polymerase promoter. After, or during, the RPA reaction, an RNA polymerase is added that will produce RNA from the double-stranded DNA templates. The amplified target RNA can then in turn be detected by, for example, a CRISPR system. In this way target DNA can be detected using the embodiments disclosed herein. RPA reactions can also be used to amplify target RNA. The target RNA is first converted to cDNA using a reverse transcriptase, followed by second strand DNA synthesis, at which point the RPA reaction proceeds as outlined above.

The methods and components described herein may be used for multiplex amplification. Multiplex amplification generally refers to the amplification of more than one nucleic acid of interest (e.g., amplification or more than one target sequence). For example, multiplex amplification can refer to amplification of multiple sequences from the same sample or amplification of one of several sequences in a sample. Multiplex amplification also may refer to amplification of one or more sequences present in multiple samples either simultaneously or instep-wise fashion. For example, a multiplex amplification may be used for amplifying least two target sequences that are capable of being amplified (e.g., the amplification reaction comprises the appropriate primers and enzymes to amplify at least two target sequences). In some instances, an amplification reaction may be prepared to detect at least two target sequences, but only one of the target sequences may be present in the sample being tested, such that both sequences are capable of being amplified, but only one sequence is amplified. In some instances, where two target sequences are present, an amplification reaction may result in the amplification of both target sequences. A multiplex amplification reaction may result in the amplification of one, some, or all of the target sequences for which it comprises the appropriate primers and enzymes. In some instances, an amplification reaction may be prepared to detect two sequences with one pair of primers, where one sequence is a target sequence and one sequence is a control sequence (e.g., a synthetic sequence capable of being amplified by the same primers as the target sequence and having a different spacer base or sequence than the target). In some instances, an amplification reaction may be prepared to detect multiple sets of sequences with corresponding primer pairs, where each set includes a target sequence and a control sequence.

Accordingly, in certain embodiments the methods disclosed herein include amplification reagents. Polymerases are proteins capable of catalyzing the specific incorporation of nucleotides to extend a 3′ hydroxyl terminus of a primer molecule, such as, for example, an amplification primer, against a nucleic acid target sequence (e.g., to which a primer is annealed). Polymerases may include, for example, thermophilic or hyperthermophilic polymerases that can have activity at an elevated reaction temperature (e.g., above 55° C., above 60° C., above 65° C., above 70° C., above 75° C., above 80° C., above 85° C., above 90° C., above 95° C., above 100° C.). A hyperthermophilic polymerase may be referred to as a hyperthermophile polymerase. A polymerase having hyperthermophilic polymerase activity may be referred to as having hyperthermophile polymerase activity. A polymerase may or may not have strand displacement capabilities. In some embodiments, a polymerase can incorporate about 1 to about 50 nucleotides in a single synthesis. For example, a polymerase may incorporate about 5, 10, 15, 20, 25, 30, 35, 40, 45 or 50 nucleotides in a single synthesis. In some embodiments, a polymerase, can incorporate 20 to 40 nucleotides in a single synthesis. In some embodiments, a polymerase, can incorporate up to 50 nucleotides in a single synthesis. In some embodiments, a polymerase, can incorporate up to 40 nucleotides in a single synthesis. In some embodiments, a polymerase, can incorporate up to 30 nucleotides in a single synthesis. In some embodiments, a polymerase, can incorporate up to 20 nucleotides in a single synthesis.

In some embodiments, amplification reaction components comprise one or more DNA polymerases. In some embodiments, amplification reaction components comprise one or more DNA polymerases comprising: 9° N DNA polymerase; 9° Nm™ DNA polymerase; THERMINATOR™ DNA Polymerase; THERMINATOR™ II DNA Polymerase; THERMINATOR™ III DNA Polymerase; THERMINATOR™ gamma. DNA Polymerase; Bst DNA polymerase; Bst DNA polymerase (large fragment); Phi29 DNA polymerase, DNA polymerase I (E. coli), DNA polymerase I, large (Klenow) fragment; Klenow fragment (3′-5′ exo-); T4 DNA polymerase; T7 DNA polymerase; DEEP VENTR™ (exo-) DNA Polymerase; D DEEP VENTR™ DNA Polymerase; DYNAZYME™ EXT DNA; DyNAzyme™ II Hot Start DNA Polymerase; PHUSION™ High-Fidelity DNA Polymerase; VENTR® DNA Polymerase; VENTR® (exo-) DNA Polymerase; REPLIPHI™ Phi29 DNA polymerase; EquiPhi29 DNA polymerase; rBst DNA Polymerase, large fragment (ISOTHERM™ DNA polymerase); MASTERAMP™ AMPLITHERM™ DNA Polymerase; Tag DNA polymerase; Tth DNA polymerase; Tfl DNA polymerase; Tgo DNA polymerase;SP6 DNA polymerase; Tbr DNA polymerase; DNA polymerase Beta; and ThermoPhi DNA polymerase.

In some embodiments, amplification reaction components comprise one or more hyperthermophile DNA polymerases. Generally, hyperthermophile DNA polymerases are thermostable at high temperatures. For example, a hyperthermophile DNA polymerase may have a half-life of about 5 to 10hours at 95 degrees Celsius and a half-life of about 1 to 3 hours at 100degrees Celsius. In some embodiments, amplification reaction components comprise one or more hyperthermophile DNA polymerases from Archaea. In some embodiments, amplification reaction components comprise one or more hyperthermophile DNA polymerases from Thermococcus. In some embodiments, amplification reaction components comprise one or more hyperthermophile DNA polymerases from Thermococcaceaen archaean. In some embodiments, amplification reaction components comprise one or more hyperthermophile DNA polymerases from Pyrococcus. In some embodiments, amplification eaction components comprise one or more hyperthermophile DNA polymerases from Methanococcaceae. In some embodiments, amplification reaction components comprise one or more hyperthermophile DNA polymerases from Methanococcus. In some embodiments, amplification reaction components comprise one or more hyperthermophile DNA polymerases from Thermus. In some embodiments, amplification reaction components comprise one or more hyperthermophile DNA polymerases from Thermus thermophiles.

Other components or reagents useful for amplification of nucleic acids are described herein. For example, an amplification reagent as described herein may include a buffer, such as a Tris buffer. A Tris buffer may be used at any concentration appropriate for the desired application or use, for example including, but not limited to, a concentration of 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 25 mM, 50 mM, 75 mM, 1 M, or the like. One of skill in the art will be able to determine an appropriate concentration of a buffer such as Tris for use with the present invention.

A salt, such as magnesium chloride (MgCl₂), potassium chloride(KCl), or sodium chloride (NaCl), may be included in an amplification reaction, such as PCR, in order to improve the amplification of nucleic acid fragments. Although the salt concentration will depend on the particular reaction and application, in some embodiments, nucleic acid fragments of a particular size may produce optimum results at particular salt concentrations. Larger products may require altered salt concentrations, typically lower salt, in order to produce desired results, while amplification of smaller products may produce better results at higher salt concentrations. One of skill in the art will understand that the presence and/or concentration of a salt, along with alteration of salt concentrations, may alter the stringency of a biological or chemical reaction, and therefore any salt may be used that provides the appropriate conditions for a reaction of the present disclosure.

Other components of a biological or chemical reaction may include a cell lysis component in order to break open or lyse a cell for analysis of the materials therein. A cell lysis component may include, but is not limited to, a detergent, a salt as described above, such as NaCl, KCl, ammonium sulfate [(NH₄)₂SO₄], or others. Detergents that may be appropriate include Triton X-100, sodium dodecyl sulfate (SDS), CHAPS(3-[(3-cholamidopropyl)dimethylammonio]-1-propanesulfonate), ethyl trimethyl ammonium bromide, nonyl phenoxypolyethoxylethanol (NP-40). Concentrations of detergents may depend on the particular application, and may be specific to the reaction in some cases. Amplification reactions may include dNTPs and nucleic acid primers used at any concentration appropriate for the invention, such as including, but not limited to, a concentration of 100 nM, 150 nM, 200 nM, 250 nM, 300 nM,350 nM, 400 nM, 450 nM, 500 nM, 550 nM, 600 nM, 650 nM, 700 nM, 750 nM,800 nM, 850 nM, 900 nM, 950 nM, 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90mM, 100 mM, 150 mM, 200 mM, 250 mM, 300 mM, 350 mM, 400 mM, 450 mM, 500mM, or the like. Likewise, a polymerase useful in accordance with the disclosure may be any specific or general polymerase known in the art and useful for the invention, including Taq polymerase, Q5 polymerase, or the like.

In some embodiments, amplification reagents as described herein may be appropriate for use in hot-start amplification. Hot start amplification may be beneficial in some embodiments to reduce or eliminate dimerization of adaptor molecules or oligonucleotides, or to otherwise prevent unwanted amplification products or artifacts and obtain optimum amplification of the desired product. Many components described herein for use in amplification may also be used in hot-start amplification. In some embodiments, reagents or components appropriate for use with hot-start amplification may be used in place of one or more of the composition components as appropriate. For example, a polymerase or other reagent may be used that exhibits a desired activity at a particular temperature or other reaction condition. In some embodiments, reagents may be used that are designed or optimized for use in hot-start amplification, for example, a polymerase may be activated after transposition or after reaching a particular temperature. Such polymerases may be antibody-based or aptamer-based. Polymerases as described herein are known in the art. Examples of such reagents may include, but are not limited to, hot-start polymerases, hot-start dNTPs and photo-caged dNTPs. Such reagents are known and available in the art. One of skill in the art will be able to determine the optimum temperatures as appropriate for individual reagents.

Amplification of nucleic acids may be performed using specific thermal cycle machinery or equipment, and may be performed in single reactions or in bulk, such that any desired number of reactions may be performed simultaneously. In some embodiments, amplification may be performed using microfluidic or robotic devices, or may be performed using manual alteration in temperatures to achieve the desired amplification. In some embodiments, optimization may be performed to obtain the optimum reaction conditions for the particular application or materials. One of skill in the art will understand and be able to optimize reaction conditions to obtain sufficient amplification.

The primers may be modified to protect against exonuclease activity. In some instances, exonuclease inhibitors are used to inhibit exonuclease activity. In certain embodiments, a combination of modified primers and exonuclease inhibitors may be used.

In certain embodiments, the nuclease activity is inhibited by exonuclease inhibitors comprising: citrate, citrate acid; MES, 2-morpholin-4-ylethanesulfonate; PV6R, pontacyl violet 6R; PCMPS, p-chloromercuriphenyl sulfonate; NCA, 7-nitroindole-2-carboxylic acid; DR396, 4-[(4,6-dichloro-1,3,5-triazin-2-yl)amino]-2-(3-hydroxy-6-oxoxanthen-9-yl)benzoic acid; DTNB, 5,5′-dithiobis(2-nitrobenzoic acid); ATA, aurintricarboxylic acid; FDCO, fmoc-D-Cha-OH, Mirin, PFM01/SML1735, PFM03, PFM39/SML1839 or combinations thereof.

In certain embodiments, the primers comprise one or more modifications to protect against exonuclease activity. In certain embodiments, the primers comprise phosphorothiolate bonds at the 5′ or 3′ end to protect against exonuclease activity. Other examples include: “linkers” at either the 5′ or 3′ end, including fluorescent tags, biotin or other affinity labels, or reactive groups, poly tails, e.g. poly-U tails; 2′-O-modified riboses, are both stable to spontaneous hydrolysis and offer strong resistance to exonuclease activity., e.g. 2′-O-methyl and 2′-O-methoxyethyl (MOE) nucleosides, which contain bulky substituents off the sugar ring; Inverted dT or ddT can be incorporated at the 3′ end of an oligonucleotide, leading to a 3′-3′ linkage that inhibit degradation by 3′ exonucleases and extension by DNA polymerases. In addition, placing an inverted, 2′,3′ dideoxy-dT base (5′ Inverted ddT) at the 5′ end of an oligonucleotide prevents spurious ligations and may protect against some forms of enzymatic degradation. Phosphorylation of the 3′ end of oligonucleotides will inhibit degradation by some 3′-exonucleases; phosphoramidite C3 Spacer can be incorporated internally, or at either end of an oligo to introduce a long hydrophilic spacer arm for the attachment of fluorophores or other pendent groups.

In some embodiments, a primer comprises modified nucleotides. A nucleotide (or base) may be modified according to any modification described herein or known in the art. Modifications may include those made during primer synthesis and/or may include post-synthetic modifications. Modifications may include internal modifications, modifications and the 3′ end of a primer, and/or modifications at the 5′ end of a primer. In some embodiments, a primer comprises a mixture of modified and unmodified nucleotides. In some embodiments, a primer comprises unmodified nucleotides. In some embodiments, a primer consists essentially of unmodified nucleotides. In some embodiments, a primer consists of unmodified nucleotides.

Modifications and modified bases may include, for example, phosphorylation, (e.g., 3′ phosphorylation, 5′ phosphorylation); attachment chemistry or linkers modifications (e.g., ACRYDITE™, adenylation, azide (NHS ester), digoxigenin (NHS ester), cholesteryl-TEG, I-LINKER™ amino modifiers (e.g., amino modifier C6, amino modifier C12, amino modifier C6 dT, UNI-LINK™ amino modifier), alkynes (e.g., 5′ hexynyl, 5-octadiynyl dU), biotinylation (e.g., biotin, biotin (azide), biotin dT, biotin-TEG, dual biotin, PC biotin, desthiobiotin-TEG), thiol modifications (e.g., thiol modifier C3 S-S, dithiol, thiol modifier C6 S-S)); fluorophores (e.g., FREEDOM™ Dyes, Alexa FLUOR® Dyes, LI-COR IRDYES®, ATTO™ Dyes, Rhodamine Dyes, WellRED Dyes, 6-FAM (azide), TEXAS RED®-X (NHS ester), LIGHTCYCLER® 640 (NHS ester), Dy 750 (NHS ester)); IOWA BLACK® dark quenchers modifications (e.g., IOWA BLACK® FQ, IOWA BLACK® RQ); dark quenchers modifications (e.g., BLACK HOLE QUENCHER®-1, BLACK HOLE QUENCHER®-2, Dabcyl); spacers (C3 spacer, PC spacer, hexanediol, spacer 9, spacer 18, 1′,2′-dideoxyribose (dSpacer); modified bases (e.g., 2-aminopurine, 2,6-diaminopurine (2-amino-dA), 5-bromo dU, deoxyUridine, inverted dT, inverted dideoxy-T, dideoxy-C, 5-methyl dC, deoxylnosine, Super T®, Super G®, locked nucleic acids (LNA's), 5-nitroindole, 2′-O-methyl RNA bases, hydroxmethyl dC, UNA unlocked nucleic acid (e.g., UNA-A, UNA-U, UNA-C, UNA-G), Iso-dC, Iso-dG, Fluoro C, Fluoro U, Fluoro A, Fluoro G); phosphorothioate bonds modifications (e.g., phosphorothioated DNA bases, phosphorothioated RNA bases, phosphorothioated 2′ O-methyl bases, phosphorothioated LNA bases); and click chemistry modifications. In some embodiments, modifications and modified bases include uracil bases, ribonucleotide bases, O-methyl RNA bases, phosphorothioate linkages, 3′ phosphate groups, spacer bases (such as C3 spacer or other spacer bases). For example, a primer may comprise one or more O-methyl RNA bases (e.g., 2′-O-methyl RNA bases). 2′-O-methyl RNA generally is a post-transcriptional modification of RNA found in tRNA and other small RNAs. Primers can be directly synthesized that include 2′-O-methyl RNA bases. This modification can, for example, increase Tₘ of RNA:RNA duplexes and provide stability in the presence of single-stranded ribonucleases and DNases. 2′-O-methyl RNA bases may be included in primers, for example, to increase stability and binding affinity to a target sequence. In some embodiments, a primer may comprise one or more phosphorothioate linkages (e.g., phosphorothioate bond modifications). A phosphorothioate (PS) bond substitutes a sulfur atom for a non-bridging oxygen in the phosphate backbone of a primer. This modification typically renders the internucleotide linkage resistant to nuclease degradation. Phosphorothioate bonds may be introduced between about the last 3 to 5 nucleotides at the 5′-end or the 3′-end of a primer to inhibit exonuclease degradation, for example. Phosphorothioate bonds included throughout an entire primer can help reduce attack by endonucleases, in certain instances. In some embodiments, a primer may comprise a 3′ phosphate group. 3′ phosphorylation can inhibit degradation by certain 3′-exonucleases and can be used to block extension by DNA polymerases, in certain instances. In some embodiments, a primer may comprise one or more spacer bases (e.g., one or more C3 spacers). A C3 spacer phosphoramidite can be incorporated internally or at the 5′-end of a primer. Multiple C3 spacers may be added at either end of a primer to introduce a long hydrophilic spacer arm for the attachment of fluorophores or other pendent groups, for example.

In some embodiments, a primer comprises DNA bases. In some embodiments, a primer comprises RNA bases. In some embodiments, a primer comprises a mixture of DNA bases and RNA bases. DNA bases may be modified or unmodified. RNA bases may be modified or unmodified. In some embodiments, a primer consists essentially of DNA bases (e.g., modified DNA bases and/or unmodified DNA bases). In some embodiments, a primer consists of DNA bases (e.g., modified DNA bases and/or unmodified DNA bases). In some embodiments, a primer consists essentially of unmodified DNA bases. In some embodiments, a primer consists of unmodified DNA bases. In some embodiments, a primer consists essentially of modified DNA bases. In some embodiments, a primer consists of modified DNA bases. In some embodiments, a primer consists essentially of RNA bases (e.g., modified RNA bases and/or unmodified RNA bases). In some embodiments, a primer consists of RNA bases (e.g., modified RNA bases and/or unmodified RNA bases). In some embodiments, a primer consists essentially of unmodified RNA bases. In some embodiments, a primer consists of unmodified RNA bases. In some embodiments, a primer consists essentially of modified RNA bases. In some embodiments, a primer consists of modified RNA bases.

In some embodiments, a primer comprises no RNA bases. In some embodiments, a primer comprises no RNA bases at the 3′ end. In some embodiments, a primer comprises a DNA base (or modified DNA base) at the 3′ end. In some embodiments, a primer is not a chimeric primer. A chimeric primer is a primer comprising DNA and RNA bases. In some embodiments, a primer is a homogeneous primer. In some embodiments, a primer is a homogeneous DNA primer. A homogeneous DNA primer may comprise unmodified DNA bases, modified DNA bases, or a mixture of modified DNA bases and unmodified DNA bases, and generally do not include RNA bases.

### Nuclease Compositions

Compositions of the disclosure include at least one gene editing agent or sequence specific nucleases, comprising CRISPR-associated nucleases such as Cas9 and Cpf1 gRNAs, Argonaute family of endonucleases, clustered regularly interspaced short palindromic repeat (CRISPR) nucleases, zinc-finger nucleases (ZFNs), transcription activator-like effector nucleases (TALENs), meganucleases, other endo- or exo-nucleases, restriction enzymes or combinations thereof. See Schiffer, 2012, J Virol 88(17):8920-8936, incorporated by reference.

The composition can also include Cas13a-the first naturally-occurring CRISPR system that targets only RNA. The Class 2 type VI-A CRISPR-Cas effector “Casi3a” demonstrates an RNA-guided RNase function. Cas13from the bacterium Leptotrichia shahii provides interference against RNA phage. In vitro biochemical analysis show that Cas13a is guided by a single crRNA and can be programmed to cleave ssRNA targets carrying complementary protospacers. In bacteria, Cas13a can be programmed to knock down specific mRNAs. Cleavage is mediated by catalytic residues in the two conserved HEPN domains, mutations in which generate catalytically inactive RNA-binding proteins. These results demonstrate the capability of Cas13a as a new RNA-targeting tools.

Cas13a can be programmed to cleave particular RNA sequences in bacterial cells. The RNA-focused action of Cas13a complements the CRISPR-Cas9 system, which targets DNA, the genomic blueprint for cellular identity and function. The ability to target only RNA, which helps carry out the genomic instructions, offers the ability to specifically manipulate RNA in a high-throughput manner-and manipulate gene function more broadly.

CRISPR/Cpf1 is a DNA-editing technology analogous to the CRISPR/Cas9 system, characterized in 2015 by Feng Zhang's group from the Broad Institute and MIT. Cpf1 is an RNA-guided endonuclease of a class II CRISPR/Cas system. This acquired immune mechanism is found in Prevotella and Francisella bacteria. It prevents genetic damage from viruses. Cpf1 genes are associated with the CRISPR locus, coding for an endonuclease that use a guide RNA to find and cleave viral DNA. Cpf1 is a smaller and simpler endonuclease than Cas9, overcoming some of the CRISPR/Cas9 system limitations.

As referenced above, Argonaute is another potential gene editing system. Argonautes are a family of endonucleases that use 5′ phosphorylated short single-stranded nucleic acids as guides to cleave targets (Swarts, D. C. et al. The evolutionary journey of Argonaute proteins. Nat. Struct. Mol. Biol. 21, 743-753 (2014)). Similar to Cas9, Argonautes have key roles in gene expression repression and defense against foreign nucleic acids (Swarts, D. C. etal. Nat. Struct. Mol. Biol. 21, 743-753 (2014); Makarova, K. S., et al. Biol. Direct 4, 29 (2009). Molloy, S. Nat. Rev. Microbiol. 11, 743 (2013); Vogel, J. Science 344, 972-973 (2014). Swarts, D. C. et al. Nature 507, 258-261 (2014); Olovnikov, I., et al. Mol. Cell 51, 594-605 (2013)). However, Argonautes differ from Cas9 in many ways Swarts, D. C. et al. The evolutionary journey of Argonaute proteins. Nat. Struct. Mol. Biol. 21, 743-753 (2014)). Cas9 only exist in prokaryotes, whereas Argonautes are preserved through evolution and exist in virtually all organisms; although most Argonautes associate with single-stranded (ss)RNAs and have a central role in RNA silencing, some Argonautes bind ssDNAs and cleave target DNAs (Swarts, D. C. et al. Nature 507, 258-261 (2014); Swarts, D. C. et al. Nucleic Acids Res. 43, 5120-5129 (2015)). guide RNAs must have a 3′ RNA-RNA hybridization structure for correct Cas9 binding, whereas no specific consensus secondary structure of guides is required for Argonaute binding; whereas Cas9 can only cleave a target upstream of a PAM, there is no specific sequence on targets required for Argonaute. Once Argonaute and guides bind, they affect the physicochemical characteristics of each other and work as a whole with kinetic properties more typical of nucleic-acid-binding proteins (Salomon, W. E., et al. Cell 162, 84-95 (2015)).

CRISPR Associated Endonucleases: CRISPR (Clustered Regularly Interspaced Short Palindromic Repeats) is found in bacteria and is believed to protect the bacteria from phage infection. It has recently been used as a means to alter gene expression in eukaryotic DNA, but has not been proposed as an anti-viral therapy or more broadly as a way to disrupt genomic material. Rather, it has been used to introduce insertions or deletions as a way of increasing or decreasing transcription in the DNA of a targeted cell or population of cells. See for example, Horvath et al., Science (2010) 327:167-170; Terns et al., Current Opinion in Microbiology (2011) 14:321-327; Bhaya et al., Annu Rev Genet (2011) 45:273-297; Wiedenheft et al., Nature (2012) 482:331-338); Jinek M et al., Science (2012) 337:816-821; Cong L et al., Science (2013) 339:819-823; Jinek M et al., (2013) eLife 2:e00471; Mali P et al. (2013) Science 339:823-826; Qi L S et al. (2013) Cell 152:1173-1183; Gilbert L A et al. (2013) Cell 154:442-451; Yang H et al. (2013) Cell 154:1370-1379; and Wang H et al. (2013) Cell 153:910-918).

CRISPR methodologies employ a nuclease, CRISPR-associated (Cas), that complexes with small RNAs as guides (gRNAs) to cleave DNA in a sequence-specific manner upstream of the protospacer adjacent motif (PAM) in any genomic location. CRISPR may use separate guide RNAs known as the crRNA and tracrRNA. These two separate RNAs have been combined into a single RNA to enable site-specific mammalian genome cutting through the design of a short guide RNA. Cas and guide RNA (gRNA) may be synthesized by known methods. Cas/guide-RNA (gRNA) uses a non-specific DNA cleavage protein Cas, and an RNA oligonucleotide to hybridize to target and recruit the Cas/gRNA complex. See Chang et al., 2013, Cell Res. 23:465-472; Hwang et al., 2013, Nat. Biotechnol. 31:227-229; Xiao et al., 2013, Nucl. Acids Res. 1-11.

In general, the CRISPR/Cas proteins comprise at least one RNA recognition and/or RNA binding domain. RNA recognition and/or RNA binding domains interact with guide RNAs. CRISPR/Cas proteins can also comprise nuclease domains (i.e., DNase or RNase domains), DNA binding domains, helicase domains, RNase domains, protein-protein interaction domains, dimerization domains, as well as other domains.

In embodiments. the CRISPR/Cas-like protein can be a wild type CRISPR/Cas protein, a modified CRISPR/Cas protein, or a fragment of a wild type or modified CRISPR/Cas protein. The CRISPR/Cas-like protein can be modified to increase nucleic acid binding affinity and/or specificity, alter an enzymatic activity, and/or change another property of the protein. For example, nuclease (i.e., DNase, RNase) domains of the CRISPR/Cas-like protein can be modified, deleted, or inactivated. Alternatively, the CRISPR/Cas-like protein can be truncated to remove domains that are not essential for the function of the fusion protein. The CRISPR/Cas-like protein can also be truncated or modified to optimize the activity of the effector domain of the fusion protein.

In some embodiments, the CRISPR/Cas-like protein can be derived from a wild type Cas9 protein or fragment thereof. In other embodiments, the CRISPR/Cas-like protein can be derived from modified Cas9 protein. For example, the amino acid sequence of the Cas9 protein can be modified to alter one or more properties (e.g., nuclease activity, affinity, stability, etc.) of the protein. Alternatively, domains of the Cas9 protein not involved in RNA-guided cleavage can be eliminated from the protein such that the modified Cas9 protein is smaller than the wild type Cas9 protein.

The composition also encompasses isolated nucleic acids encoding one or more elements of the CRISPR-Cas system. For example, in one embodiment, the composition comprises an isolated nucleic acid encoding at least one of the guide nucleic acid molecule and a CRISPR-associated (Cas) peptide, or functional fragment or derivative thereof.

Three types (I-III) of CRISPR systems have been identified. CRISPR clusters contain spacers, the sequences complementary to antecedent mobile elements. CRISPR clusters are transcribed and processed into mature CRISPR RNA (crRNA). In embodiments, the CRISPR/Cas system can be a type I, a type II, or a type III system. Non-limiting examples of suitable CRISPR/Cas proteins include Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, CjCas9, SpCas9, Cas13, Cas14, Cpf1, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, Cu1966 or combinations thereof.

A variety of CRISPR systems have been generated for efficient gene editing. The Cas9 variant CjCas9, derived from Campylobacter jejuni, is composed of 984 amino acid residues (2.95 kbp) and has been used for efficient gene editing in vitro and in vivo. CjCas9 is highly specific and cuts only a limited number of sites in the genomes of mouse or human. Delivered through adeno-associated virus (AAV), it has been shown to induce targeted mutations at high frequencies in retinal pigment epithelium (RPE) cells or mouse muscle cells.

Phage-assisted continuous evolution was used to develop an SpCas9 variant, xCas9(3.7), which recognizes a broader range of protospacer adjacent motifs (PAMs) (Rees, H. A. and Liu, D. R. (2018) Base editing: precision chemistry on the genome and transcriptome of living cells. Nat. Rev. Genet. 19, 770-788). xCas9 possesses a higher DNA specificity and editing efficiency, lower off-target activity, and broader PAM compatibility (including NG, GAA, and GAT) than does SpCas9, from which it is derived (Hu, J. H. et al. (2018) Evolved Cas9 variants with broad PAM compatibility and high DNA specificity. Nature 556, 57-63).

In one embodiment, the RNA-guided endonuclease is derived from a type II CRISPR/Cas system. The CRISPR-associated endonuclease, Cas9, belongs to the type II CRISPR/Cas system and has strong endonuclease activity to cut target DNA. Cas9 is guided by a mature crRNA that contains about 20 base pairs (bp) of unique target sequence (called spacer) and a trans-activated small RNA (tracrRNA) that serves as a guide for ribonuclease III-aided processing of pre-crRNA. The crRNA:tracrRNA duplex directs Cas9 to target DNA via complementary base pairing between the spacer on the crRNA and the complementary sequence (called protospacer) on the target DNA. Cas9 recognizes a trinucleotide (NGG) protospacer adjacent motif (PAM) to specify the cut site (the 3rd nucleotide from PAM). The crRNA and tracrRNA can be expressed separately or engineered into an artificial fusion small guide RNA (sgRNA) via a synthetic stem loop (AGAAAU) to mimic the natural crRNA/tracrRNA duplex. Such sgRNA, like shRNA, can be synthesized or in vitro transcribed for direct RNA transfection or expressed from U6 or H1-promoted RNA expression vector, although cleavage efficiencies of the artificial sgRNA are lower than those for systems with the crRNA and tracrRNA expressed separately.

The CRISPR-associated endonuclease Cas9 nuclease can have a nucleotide sequence identical to the wild type Streptococcus pyogenes sequence. The CRISPR-associated endonuclease may be a sequence from other species, for example other Streptococcus species, such as thermophiles. The Cas9 nuclease sequence can be derived from other species including, but not limited to: Nocardiopsis dassonvillei, Streptomyces pristinaespiralis, Streptomyces viridochromogenes, Streptomyces roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Mierocystis aeruginosa, Synechococcus sp., Acetohalobium arabaticum, Ammonifex degensii, Caldicelulosiruptor becscii, Candidatus desulforudis, Clostridium botulinum, Clostridium difficle, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer,, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina. Pseudomonas aeruginosa, Escherichia coli, or other sequenced bacteria genomes and archaea, or other prokaryotic microorganisms may also be a source of the Cas9 sequence utilized in the embodiments disclosed herein.

The wild type Streptococcus pyogenes Cas9 sequence can be modified. The nucleic acid sequence can be codon optimized for efficient expression in plant cells Alternatively, the Cas9 nuclease sequence can be for example, the sequence contained within a commercially available vector such as PX330 or PX260 from Addgene (Cambridge, MA). In some embodiments, the Cas9 endonuclease can have an amino acid sequence that is a variant or a fragment of any of the Cas9 endonuclease sequences of Genbank accession numbers KM099231.1 GI:669193757; KM099232.1 GI:669193761; or KM099233.1 GI:669193765 or Cas9 amino acid sequence of PX330 or PX260 (Addgene, Cambridge, MA). The Cas9 nucleotide sequence can be modified to encode biologically active variants of Cas9, and these variants can have or can include, for example, an amino acid sequence that differs from a wild type Cas9 by virtue of containing one or more mutations (e.g., an addition, deletion, or substitution mutation or a combination of such mutations). One or more of the substitution mutations can be a substitution (e.g., a conservative amino acid substitution). For example, a biologically active variant of a Cas9 polypeptide can have an amino acid sequence with at least or about 50% sequence identity (e.g., at least or about 50%, 55%, 60%, 65%, 70%, 75%, 80%, 85%, 90%, 95%, 97%, 98%, or 99% sequence identity) to a wild type Cas9 polypeptide. Conservative amino acid substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine. The amino acid residues in the Cas9 amino acid sequence can be non-naturally occurring amino acid residues. Naturally occurring amino acid residues include those naturally encoded by the genetic code as well as non-standard amino acids (e.g., amino acids having the D-configuration instead of the L-configuration). The present peptides can also include amino acid residues that are modified versions of standard residues (e.g. pyrrolysine can be used in place of lysine and selenocysteine can be used in place of cysteine). Non-naturally occurring amino acid residues are those that have not been found in nature, but that conform to the basic formula of an amino acid and can be incorporated into a peptide. These include D-alloisoleucine(2R,3 S)-2-amino-3-methylpentanoic acid and L-cyclopentyl glycine (S)-2-amino-2-cyclopentyl acetic acid. For other examples, one can consult textbooks or the worldwide web (a site currently maintained by the California Institute of Technology displays structures of non-natural amino acids that have been successfully incorporated into functional proteins).

The Cas9 nuclease sequence can be a mutated sequence. For example, the Cas9 nuclease can be mutated in the conserved HNH and RuvC domains, which are involved in strand specific cleavage. For example, an aspartate-to-alanine (D10A) mutation in the RuvC catalytic domain allows the Cas9 nickase mutant (Cas9n) to nick rather than cleave DNA to yield single-stranded breaks, and the subsequent preferential repair through HDR can potentially decrease the frequency of unwanted indel mutations from off-target double-stranded breaks.

The Cas9 can be an orthologous. Six smaller Cas9 orthologues have been used and reports have shown that Cas9 from Staphylococcus aureus (SaCas9) can edit the genome with efficiencies similar to those of SpCas9, while being more than 1 kilobase shorter.

In addition to the wild type and variant Cas9 endonucleases described, embodiments of the disclosure also encompass CRISPR systems including newly developed “enhanced-specificity” S. pyogenes Cas9 variants (eSpCas9), which dramatically reduce off target cleavage. These variants are engineered with alanine substitutions to neutralize positively charged sites in a groove that interacts with the non-target strand of DNA. This aim of this modification is to reduce interaction of Cas9 with the non-target strand, thereby encouraging re-hybridization between target and non-target strands. The effect of this modification is a requirement for more stringent Watson-Crick pairing between the gRNA and the target DNA strand, which limits off-target cleavage (Slaymaker, I. M. et al. (2015) DOI:10.1126/science.aad5227).

Three variants found to have the best cleavage efficiency and fewest off-target effects: SpCas9(K855A), SpCas9(K810A/K1003A/R1060A) (a.k.a. eSpCas9 1.0), and SpCas9(K848A/K1003A/R1060A) (a.k.a. eSPCas9 1.1) are employed in the compositions. The invention is by no means limited to these variants, and also encompasses all Cas9 variants (Slaymaker, I. M. et al. (2015)).

The present disclosure also includes another type of enhanced specificity Cas9 variant, “high fidelity” spCas9 variants (HF-Cas9) (Kleinstiver, B. P. et al., 2016, Nature. DOI: 10.1038/nature16526).

As used herein, the term “Cas” is meant to include all Cas molecules comprising variants, mutants, orthologues, high-fidelity variants and the like.

In one embodiment, the Cas peptide is Cas9 or a variant thereof. In one embodiment, the Cas9 variant comprises one or more point mutations, relative to wildtype Streptococcus pyogenes Cas9 (spCas9), selected from the group consisting of: R780A, K810A, K848A, K855A, H982A, K1003A, R1060A, D1135E, N497A, R661A, Q695A, Q926A, L169A, Y450A, M495A, M694A, and M698A. In one embodiment, the Cas peptide is Cpf1 or a variant thereof.

Multiplex Gene-Editing: CRISPR has the potential to create mutations simultaneously at more than one genomic site by using multiple sgRNAs, in any organism. CRISPR/Cas9 has also been used for multiplex gene editing, which enables the rapid stacking of multiple traits in an elite variety background (Yin, K. et al. (2017) Progress and prospects in plant genome editing. Nat. Plants 3, 17107). Multiplex gene editing also provides a powerful tool for targeting multiple members of multigene families. It can be achieved in two ways, by either constructing multiple gRNA expression cassettes in separate vectors or assembling various sgRNAs in a single vector (Wang, C. et al. (2019) Clonal seeds from hybrid rice by simultaneous genome engineering of meiosis and fertilization genes. Nat Biotechnol. 37, 283-28).

CRISPR Interference and Activation: The CRISPR interfering (CRISPRi) system is used as an orthogonal system in a variety of living organisms; the requirements are only a coexpression of a catalytically inactive Cas9 protein and a modified sgRNA, designed with a complementary region to any gene of interest. The CRISPRi system is derived from the S. pyogenes CRISPR pathway. The complex comprising Cas9 and sgRNA binds to DNA elements complementary to the sgRNA and causes a steric block that stops transcript elongation by RNA polymerase, so repressing the target gene. Therefore, CRISPRi has been considered as an effective and precise genome-targeting platform for transcription control without changing the target DNA sequence (Larson, M. H. et al. (2013) CRISPR interference (CRISPRi) for sequence-specific control of gene expression. Nat. Protoc. 8, 2180-2196). dCas9 is a useful and robust tool for the regulation of transcription levels of any target gene. The gRNA directs the binding of dCas9 to any genomic locus that can efficiently stop the progress of RNA polymerase to the downstream gene.

Both CRISPR-based gene editing and transcriptional regulation benefit from multiplexing. By producing multiple gRNAs and a Cas protein in vivo, layered genetic circuits that control cellular behavior or modulate metabolic pathways with the simultaneous editing, activation, and downregulation of multiple target genes is accomplished (Nielsen. A. A. & Voigt, C. A. Multi--input CRISPR/Cas genetic circuits that interface host regulatory networks. Mol. Syst. Biol. 10. 763-763 (2014). Lan, J., etal., Combinatorial metabolic engineering using an orthogonal tri-functional CRISPR system. Nat. Commun. 8, 1688 (2017). Gander. M. W., etal., Digital logic circuits in yeast with CRISPR-dCas9 NOR gates. Nat. Commun. 8, 15459 (2017). Jakočiūnas, I. et a/. Multiplex metabolic pathway engineering using CRISPR/Cas9 in Saccharomyces cerevisiae. Metab. Eng. 28, 213-222 (2015)). For gene editing, CRISPRa and CRISPRi, the targeting of multiple gRNAs to a single genetic locus also enhances the efficiency of DNA editing and transcriptional control.

Guide nucleic acid molecule: In one embodiment, the composition comprises at least one isolated guide nucleic acid molecule, or fragment thereof, wherein the guide nucleic acid molecule comprises a nucleotide sequence that is complementary to one or more target sequences in the sample. In one embodiment the guide nucleic acid is a guide RNA (gRNA).

In one embodiment, the gRNA comprises a crRNA:tracrRNA duplex. In one embodiment, the gRNA comprises a stem-loop that mimics the natural duplex between the crRNA and tracrRNA. In one embodiment, the stem-loop comprises a nucleotide sequence comprising AGAAAU. For example in one embodiment, the composition comprises a synthetic or chimeric guide RNA comprising a crRNA, stem, and tracrRNA.

In certain embodiments, the composition comprises an isolated crRNA and/or an isolated tracrRNA which hybridize to form a natural duplex. For example, in one embodiment, the gRNA comprises a crRNA or crRNA precursor (pre-crRNA) comprising a targeting sequence.

In certain embodiments, the CRISPR system as provided herein can make use of a crRNA or analogous polynucleotide comprising a guide sequence, wherein the polynucleotide is an RNA, a DNA or a mixture of RNA and DNA, and/or wherein the polynucleotide comprises one or more nucleotide analogs. The sequence can comprise any structure, including but not limited to a structure of a native crRNA, such as a bulge, a hairpin or a stem loop structure. In certain embodiments, the polynucleotide comprising the guide sequence forms a duplex with a second polynucleotide sequence which can be an RNA or a DNA sequence.

The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay. Similarly, cleavage of a target RNA may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and will occur to those skilled in the art.

Guide RNA sequences according to the present disclosure can be sense or anti-sense sequences. The specific sequence of the gRNA may vary, but, regardless of the sequence, useful guide RNA sequences will be those that minimize off-target effects while achieving high efficiency and specificity of the target gene. The guide RNA sequence generally includes a proto-spacer adjacent motif (PAM). The sequence of the PAM can vary depending upon the specificity requirements of the CRISPR endonuclease used. In the CRISPR-Cas system derived from S. pyogenes, the target DNA typically immediately precedes a 5′-NGG proto-spacer adjacent motif (PAM). Thus, for the S. pyogenes Cas9, the PAM sequence can be AGG, TGG, CGG or GGG. Other Cas9 orthologues may have different PAM specificities. For example, Cas9 from S. thermophilus requires 5′-NNAGAA for CRISPR 1 and 5′-NGGNG for CRISPR3 and Neiseria meningitidis requires 5′-NNNNGATT. The specific sequence of the guide RNA may vary, but, regardless of the sequence, useful guide RNA sequences will be those that minimize off-target effects. The length of the guide RNA sequence can vary from about 20 to about 60 or more nucleotides, for example about 20, about 21, about 22, about 23, about 24, about 25, about 26, about 27, about 28, about 29, about 30, about 31, about 32, about 33, about 34, about 35, about 36, about 37, about 38, about 39, about 40, about 45, about 50, about 55, about 60 or more nucleotides.

In certain embodiments the sequence of the gRNA that is substantially complementary to the target is about 10-30 nucleotides in length. In certain embodiments, the gRNA comprises a nucleotide sequence that binds to the desired target sequence in the sample. For example, in certain embodiments, the gRNA comprises a nucleotide sequence that is substantially complementary to the target sequence, and thus binds to the target sequence. For example, in certain embodiments, a target nucleic acid sequence comprises an infectious disease agent. In certain embodiments, an infectious disease agent comprises: a virus, a latent virus, bacteria, a parasite, a fungus, a prion or combinations thereof. In certain embodiments, a target nucleic acid sequence is an abnormal (e.g. not normally found or is a mutated sequence) nucleic acid sequence. In certain embodiments, an abnormal nucleic acid sequence comprises: a mutation, an oncogene, a single nucleotide polymorphism, a sequence encoding a biomarker, a sequence encoding a tumor antigen or combinations thereof.

The guide RNA sequence can be configured as a single sequence or as a combination of one or more different sequences, e.g., a multiplex configuration. Multiplex configurations can include combinations of two, three, four, five, six, seven, eight, nine, ten, or more different guide RNAs. In certain embodiments, the composition comprises multiple different gRNA molecules, each targeted to a different target sequence. In certain embodiments, this multiplexed strategy provides for increased efficacy. These multiplex gRNAs can be expressed separately in different vectors or expressed in one single vector.

In certain embodiments, the composition comprises multiple different gRNA molecules, each targeted to a different target sequence. In certain embodiments, this multiplexed strategy provides for increased efficacy.

In certain embodiments, the RNA molecules (e.g., crRNA, tracrRNA, gRNA) may be engineered to comprise one or more modified nucleobases. For example, known modifications of RNA molecules can be found, for example, in Genes VI, Chapter 9 (“Interpreting the Genetic Code”), Lewis, ed. (1997, Oxford University Press, New York), and Modification and Editing of RNA, Grosjean and Benne, eds. (1998, ASM Press, Washington DC). Modified RNA components include the following: 2′-O-methylcytidine; N⁴-methylcytidine; N⁴-2′-O-dimethylcytidine; N⁴-acetylcytidine; 5-methylcytidine; 5,2′-0-dimethylcytidine; 5-hydroxymethylcytidine; 5-formylcytidine; 2′-O-methyl-5-formaylcytidine; 3-methylcytidine; 2-thiocytidine; lysidine; 2′-O-methyluridine; 2-thiouridine; 2-thio-2′-O-methyluridine; 3,2′-0-dimethyluridine; 3-(3-amino-3-carboxypropyl)uridine; 4-thiouridine; ribosylthymine; 5,2′-0-dimethyluridine; 5-methyl-2-thiouridine; 5-hydroxyuridine; 5-methoxyuridine; uridine 5-oxyacetic acid; uridine 5-oxyacetic acid methyl ester; 5-carboxymethyluridine; 5-methoxycarbonylmethyluridine; 5-methoxycarbonylmethyl-2′-O-methyluridine; 5-methoxycarbonylmethyl-2′-thiouridine; 5-carbamoylmethyluridine; 5-carbamoylmethyl-2′-O-methyluridine; 5-(carboxyhydroxymethyl)uridine; 5-(carboxyhydroxymethyl) uridinemethyl ester; 5-aminomethyl-2-thiouridine; 5-methylaminomethyluridine; 5-methylaminomethyl-2-thiouridine; 5-methylaminomethyl-2-selenouridine; 5-carboxymethylaminomethyluridine; 5-carboxymethylaminomethyl-2′-O-methyl- uridine; 5-carboxymethylaminomethyl-2-thiouridine; dihydrouridine; dihydroribosylthymine; 2′-methyladenosine; 2-methyladenosine; N⁶N-methyladenosine; N⁶, N⁶-dimethyladenosine; N⁶,2′-O-trimethyladenosine; 2-methylthio-N⁶N-isopentenyladenosine; N⁶-(cis-hydroxyisopentenyl)-adenosine; 2-methylthio-N⁶-(cis-hydroxyisopentenyl)-adenosine; N⁶-glycinylcarbamoyl)adenosine; N⁶-threonylcarbamoyl adenosine; N⁶-methyl-N⁶-threonylcarbamoyl adenosine; 2-methylthio-N⁶-methyl-N⁶-threonylcarbamoyl adenosine; N⁶-hydroxynorvalylcarbamoyl adenosine; 2-methylthio-N⁶-hydroxnorvalylcarbamoyl adenosine; 2′-0-ribosyladenosine (phosphate); inosine; 2′O-methyl inosine; 1-methyl inosine; 1;2′-O-dimethyl inosine; 2′-O-methyl guanosine; 1-methyl guanosine; N²-methyl guanosine; N², N²-dimethyl guanosine; N², 2′-0-dimethyl guanosine; N², N², 2′-O-trimethyl guanosine; 2′-O-ribosyl guanosine (phosphate); 7-methyl guanosine; N²;7-dimethyl guanosine; N²; N²;7-trimethyl guanosine; wyosine; methylwyosine; under-modified hydroxywybutosine; wybutosine; hydroxywybutosine; peroxywybutosine; queuosine; epoxyqueuosine; galactosyl-queuosine; mannosyl-queuosine; 7-cyano-7-deazaguanosine; arachaeosine [also called 7-formamido-7-deazaguanosine]; and 7-aminomethyl-7-deazaguanosine. The methods of the present invention or others in the art can be used to identify additional modified RNA molecules.

A guide sequence, and hence a nucleic acid-targeting guide RNA maybe selected to target any target nucleic acid sequence. In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to an RNA polynucleotide being or comprising the target sequence. In other words, the target RNA may be an RNA polynucleotide or a part of an RNA polynucleotide to which a part of the gRNA, i.e. the guide sequence, is designed to have complementarity and to which the effector function mediated by the complex comprising CRISPR effector protein and a gRNA is to be directed. In some embodiments, a target sequence is located in the nucleus or cytoplasm of a cell. The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA(tRNA), micro-RNA (miRNA), small interfering RNA (siRNA), small nuclearRNA (snRNA), double stranded RNA (dsRNA), noncoding RNA (ncRNA), long non-coding RNA (lncRNA), and small cytoplasmic RNA (scRNA). In some embodiments, the target sequence may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some embodiments, the target sequence may be a sequence within an mRNA molecule or a pre-mRNA molecule.

In some embodiments, a nucleic acid-targeting guide RNA is selected to reduce the degree of secondary structure within the RNA-targeting guide RNA. In some embodiments, about or less than about 75%, 50%, 40%,30%, 25%, 20%, 15%, 10%, 5%, 1%, or fewer of the nucleotides of the nucleic acid-targeting guide RNA participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNA fold, developed at Institute for Theoretical Chemistry at the University of Vienna, using the centroid structure prediction algorithm (see e.g., A.R. Gruber et al., 2008, Cell 106(1): 23-24; and PA Can and GM Church, 2009, Nature Biotechnology 27(12): 1151-62).

In certain embodiments, a guide RNA or crRNA may comprise, consist essentially of, or consist of a direct repeat (DR) sequence and a guide sequence or spacer sequence. In certain embodiments, the guide RNA or crRNA may comprise, consist essentially of, or consist of a direct repeat sequence fused or linked to a guide sequence or spacer sequence. In certain embodiments, the direct repeat sequence may be located upstream(i.e., 5′) from the guide sequence or spacer sequence. In other embodiments, the direct repeat sequence may be located downstream (i.e.,3′) from the guide sequence or spacer sequence.

In certain embodiments, the crRNA comprises a stem loop. In certain embodiments, the direct repeat sequence forms a stem loop.

In certain embodiments, the spacer length of the guide RNA is froml5 to 35 nt. In certain embodiments, the spacer length of the guide RNAis at least 15 nucleotides, preferably at least 18 nt, such as at leastl9, 20, 21, 22, or more nt. In certain embodiments, the spacer length is from 15 to 17 nt, e.g., 15, 16, or 17 nt, from 17 to 20 nt, e.g., 17, 18,19, or 20 nt, from 20 to 24 nt, e.g., 20, 21, 22, 23, or 24 nt, from 23to 25 nt, e.g., 23, 24, or 25 nt, from 24 to 27 nt, e.g., 24, 25, 26, or27 nt, from 27-30 nt, e.g., 27, 28, 29, or 30 nt, from 30-35 nt, e.g.,30, 31, 32, 33, 34, or 35 nt, or 35 nt or longer.

In certain embodiments, the spacer length of the guide RNA is less than 28 nucleotides. In certain embodiments, the spacer length of the guide RNA is at least 18 nucleotides and less than 28 nucleotides. In certain embodiments, the spacer length of the guide RNA is between 19 and28 nucleotides. In certain embodiments, the spacer length of the guide RNA is between 19 and 25 nucleotides. In certain embodiments, the spacer length of the guide RNA is 20 nucleotides. In certain embodiments, the spacer length of the guide RNA is 23 nucleotides. In certain embodiments, the spacer length of the guide RNA is 25 nucleotides.

In CRISPR-Cas systems, the degree of complementarity between a guide sequence and its corresponding target sequence can be about or more than about 50%, 60%, 75%, 80%, 85%, 90%, 95%, 97.5%, 99%,or 100%; a guide or RNA or sgRNA can be about or more than about 5, 10,11, 12, 13, 14, 15, 16, 17, 18, 19, 20, 21, 22, 23, 24, 25, 26, 27, 28,29, 30, 35, 40, 45, 50, 75, or more nucleotides in length; or guide or RNA or sgRNA can be less than about 75, 50, 45, 40, 35, 30, 25, 20, 15,12, or fewer nucleotides in length. In certain embodiments, the degree of complementarity between a guide sequence and its corresponding target sequence is greater than 94.5% or 95% or 95.5% or 96% or 96.5% or 97% or 97.5% or 98% or98.5% or 99% or 99.5% or 99.9%, or 100%. Off target is less than 100% or 99.9% or 99.5% or 99% or 99% or 98.5% or 98% or 97.5% or 97% or 96.5% or96% or 95.5% or 95% or 94.5% or 94% or 93% or 92% or 91% or 90% or 89% or88% or 87% or 86% or 85% or 84% or 83% or 82% or 81% or 80% complementarity between the target sequence and the guide RNA.

In certain embodiments, modulations of cleavage efficiency can be exploited by introduction of mismatches, e.g. 1 or more mismatches, such as 1 or 2 mismatches between spacer sequence and target sequence, including the position of the mismatch along the spacer/target. The more central (i.e. not 3′ or 5′) for instance a double mismatch is, the more cleavage efficiency is affected. Accordingly, by choosing a mismatch position along the spacer, cleavage efficiency can be modulated. By means of example, if less than 100% cleavage of targets is desired (e.g. in a cell population), 1 or more, such as preferably 2 mismatches between spacer and target sequence may be introduced in the spacer sequences. The more central along the spacer of the mismatch position, the lower the cleavage percentage.

In certain embodiments, the cleavage efficiency may be exploited to design single guides that can distinguish two or more targets that vary by a single nucleotide, such as a single nucleotide polymorphism (SNP), variation, or (point) mutation. The CRISPR effector may have reduced sensitivity to SNPs (or other single nucleotide variations) and continue to cleave SNP targets with a certain level of efficiency. Thus, for two targets, or a set of targets, a guide RNA maybe designed with a nucleotide sequence that is complementary to one of the targets i.e. the on-target SNP. The guide RNA is further designed to have a synthetic mismatch. As used herein a “synthetic mismatch” refers to a non-naturally occurring mismatch that is introduced upstream or downstream of the naturally occurring SNP, such as at most 5 nucleotides upstream or downstream, for instance 4, 3, 2, or 1 nucleotide upstream or downstream, at most 3 nucleotides upstream or downstream, or at most 2 nucleotides upstream or downstream, most preferablyl nucleotide upstream or downstream (i.e. adjacent the SNP). For example, the gRNAs may be used to distinguish pathogenic strains that differ by a single SNP or detect certain disease specific SNPs, such as but not limited to, disease associated SNPs, such as without limitation cancer associated SNPs.

The isolated nucleic acid molecules, including the RNA molecules (e.g., crRNA, tracrRNA, gRNA) or nucleic acids encoding the RNA molecules, may be produced by standard techniques. For example, polymerase chain reaction (PCR) techniques can be used to obtain an isolated nucleic acid containing a nucleotide sequence described herein, including nucleotide sequences encoding a polypeptide described herein. PCR can be used to amplify specific sequences from DNA as well as RNA, including sequences from total genomic DNA or total cellular RNA. Various PCR methods are described in, for example, PCR Primer: A Laboratory Manual, 2^{nd} edition, Dieffenbach and Dveksler, eds., Cold Spring Harbor Laboratory Press, 2003. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid.

The nucleic acids, e.g. gRNAs can also be chemically synthesized, either as a single nucleic acid molecule (e.g., using automated DNA synthesis in the 3′ to 5′ direction using phosphoramidite technology) or as a series of oligonucleotides. Isolated nucleic acids of the invention also can be obtained by mutagenesis of, e.g., a naturally occurring portion crRNA, tracrRNA, RNA-encoding DNA, or of a Cas9 -encoding DNA

In certain embodiments, the isolated RNA molecules are synthesized from an expression vector encoding the RNA molecule.

Restriction Enzymes: Restriction endonucleases popularly referred to as restriction enzymes, are ubiquitously present in prokaryotes. The function of restriction endonucleases is mainly protection against foreign genetic material especially against bacteriophage DNA. The other functions attributed to these enzymes are recombination and transposition. Restriction endonucleases make up the restriction-modification (R-M) systems comprised of endonuclease and methytransferase activities. The endonuclease recognizes and cleaves foreign DNA on the defined recognition sites. The methyltransferase modifies the recognition sites in the host DNA and protects it against the activity of endonucleases. The sequences in foreign DNA are generally not methylated and are subjected to restriction digestion. Each restriction enzyme recognizes a specific sequence of 4-8 nucleotides in DNA and cleaves at these sites. Endonucleases isolated by different organisms with identical recognition sites are termed isoschizomers.

The digestion activity of restriction enzymes depends on the following factors: (i) Temperature: Most endonucleases digest the target DNA at 37° C. with few exceptions. Some work at lower temperatures (˜25° C., Sma 1) while Taq I works at 65° C. (ii) Cofactors: Restriction endonucleases require certain cofactors or combination of cofactors to digest at the recognition site. All enzymes require Mg²⁺ as a cofactor for the endonuclease activity. In R-M systems with separate proteins having the restriction and methylation activities, S-adenosylmethionine (SAM) and ATP are required for methylation activity. (iii) Ionic Conditions: As mentioned previously, Mg²⁺ is required for all endonucleases but some enzymes also require ions such as Na⁺ and K⁺. (iv) Buffer systems: Most restriction enzymes are active in the pH range of 7.0-8.0. Tris-HCl, a temperature-dependent buffer, is the most commonly used buffer. (v) Methylation status of DNA: Methylation of adenine or cytidine residues affects the digestion of DNA.

Restriction Enzymes are classified based on their activity sites, required cofactors, and recognition sequences. The detailed classification and description of each type of restriction endonucleases are presented in Table 1.

| Type | Activity Site | Features |
|---|---|---|
| Type I | far from the recognition sequence | both endonuclease and methylase activities in the |
| | | same protein |
| | | require both ATP and S-adenosy1-L-methionine to |
| | | function |
| Type | close to or within short distances | only endonuclease function |
| II | from recognition sequence | require Mg²⁺ to function |
| Type | close to or within short distances | cleave asymmetric recognition sequences |
| IIA | from recognition sequence | require Mg²⁺ to function |
| Type | close to or within short distances | cleave both sides of target on both starnds |
| IIB | from recognition sequence | require Mg²⁺ to function |
| Type | close to or within short distances | both endonuclease and methylase activities in the same |
| IIC | from recognition sequence | protein |
| | | cleave both symmetric and asymmetric recognition |
| | | sequences |
| | | require Mg²⁺ to function |
| Type | close to or within short distances | interact with two copies of the recognition sequence; |
| IIE | from recognition sequence | one the actual target for cleavage and the other is the |
| | | allosteric effector |
| Type | close to or within short distances | interact with two copies of the recognition sequence; |
| IIF | from recognition sequence | one the actual target for cleavage and the other is the |
| | | allosteric effector |
| Type | close to or within short distances | both endonuclease and methylase activities in the same |
| IIG | from recognition sequence | protein |
| | | cleave both symmetric and asymmetric recognition |
| | | sequences |
| | | require Mg²⁺ to function |
| Type | close to or within short distances | cleave symmetric and asymmetric recognition |
| IIH | from recognition sequence | sequences |
| | | require Mg²⁺ to function |
| Type | close to or within short distances | acts on methylated DNA |
| IIM | from recognition sequence | require Mg²⁺ to function |
| Type | close to or within short distances | cleave symmetric recognition sequences |
| IIP | from recognition sequence | require Mg²⁺ to function |
| Type | close to or within short distances | cleave asymmetric recognition sequences |
| IIS | from recognition sequence | require Mg²⁺ to function |
| Type | close to or within short distances | cleave both symmetric and asymmetric recognition |
| IIT | from recognition sequence | sequences |
| | | require Mg²⁺ to function |
| Type | 25-27 bp outside the recognition | act on two sequences in opposite orientations with |
| III | sequence | same DNA |
| | | require both ATP and S-adenosyl-L-methionine to |
| | | function |
| Type | close to or within the recognition | act on methylated DNA |
| IV | sequence | require Mg²⁺ to function |

Examples of Type I restriction enzymes include: EcoKI, EcoAI, EcoBI, CfrAI, StyLTII, StyLTIII, and StySPI. Examples of Type II restriction enzymes include: EcoRl, BamHI, HindIII, KpnI, NotI, PstI, SmaI, XhoI. Examples of Type IIb restriction enzymes include: BcgI, Bsp24I, BaeI, CjeI, and CjePI. Examples of Type IIe restriction enzymes include: NaeI, NarI, BspMI, Hpall, Sa II, EcoRII, Eco57I, AtuBI, Cfr9I, SauBMKI, and Ksp632I. Examples of Type IIs restriction enzymes include: FokI, Alw26I, BbvI, BsrI, EarI, Hphl, Mboll, SfaNI, Tth111. Examples of Type III restriction enzymes include: EcoP15I, EcoPI, HinfIII, and StyLTI. Examples of Type IV restriction enzymes include: McrBC and Mrr systems of E. coli.

A listing of restriction enzymes can be found can be found Roberts R.J. et al. “REBASE--enzymes and genes for DNA restriction and modification.” Nucleic acids research vol. 35, Database issue (2007): D269-70. doi:10.1093/nar/gk1891, incorporated herein by reference in its entirety.

### Kits

Kits are also provided herein. The kit can include primers, sequence specific nucleases, amplification reagents and other components suitable for use in a polynucleotide amplification reaction such as divalent cations, e.g. derived from magnesium salts, deoxyribonucleotide 5′ triphosphates (dNTPs), buffering agents, etc. The kit can also include enzymes such as CRISPR/Cas, exonuclease lambda and other reagents needed to detect the nucleic acids.

Methods and kits disclosed herein may be carried out in numerous formats known in the art. In certain embodiments, the methods provided herein are carried out using solid-phase assay formats. In certain embodiments, the methods provided herein are carried out in a well of a plate with a plurality of wells, such as a multi-well plate or a multi-domain multi-well plate. The use of multi-well assay plates allows for the parallel processing and analysis of multiple samples distributed in multiple wells of a plate. Multi-well assay plates (also known as microplates or microtiter plates) can take a variety of forms, sizes and shapes (e.g., round- or flat-bottom multi-well plates). Exemplary multi-well plate formats that can be used in the methods provided herein include those found on 96-well plates (12×8 array of wells), 384-well plates (24×16 array of wells), 1536-well plate (48×32 array of well), 3456-well plates and 9600-well plates. Other formats that may be used in the methods provided herein include, but are not limited to, single or multi-well plates comprising a plurality of domains, cuvettes, microarrays etc.

The methods provided herein, when carried out in standardized plate formats can take advantage of readily available equipment for storing and moving these plates as well as readily available equipment for rapidly dispensing liquids in and out of the plates (e.g., robotic dispenser, multi-well and multi-channel pipettes, plate washers and the like).

The assays embodied herein are suitable for detecting and identifying specific target molecules in a sample. Such assays include automated, semi-automated assays and HTS (high throughput screening) assays. In HTS methods, many samples are preferably tested in parallel by robotic, automatic or semi-automatic methods so that large numbers of samples are screened simultaneously or nearly simultaneously.

### EXAMPLES

### Example 1: Nucleic Acid Detection Assay

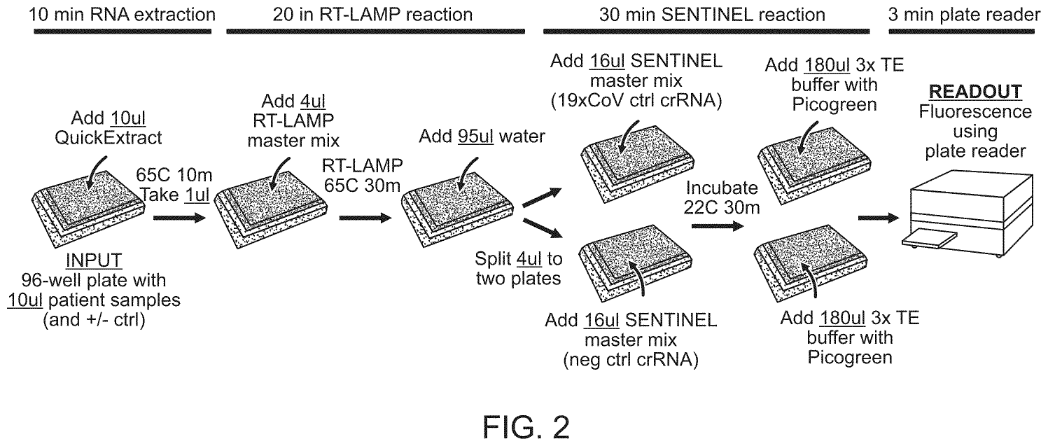

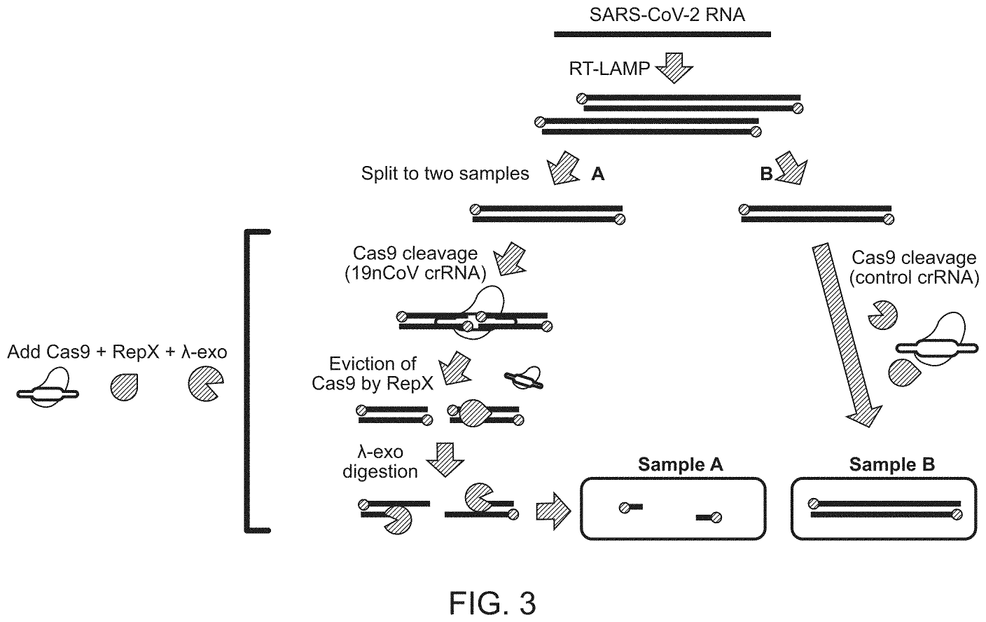

The assay, termed herein, “SENTINEL” (Specific Enzymatic Nucleic-acid Targeting with CRISPR Nucleases and Exonuclease Lambda) is rapid, inexpensive, sensitive, sequence specific, nucleic acid detection assay (FIG. 2). The method utilizes two steps to determine the presence of a nucleic acid of interest. First, isothermal amplification methods, such as, for example, loop-mediated isothermal amplification (LAMP), Recombinase polymerase amplification (RPA), PCR, and the like, amplify small amounts of either RNA or DNA from the sample to double stranded DNA (dsDNA). Inhibitors of nucleases or phosphorothiolate bonds are used to cap the ends of primers to appropriately protect the dsDNA products from exonuclease activity in the next step. Isothermal amplification methods alone are also used for nucleic acid detection, but can be non-specific due to unintended amplification of similar sequences. The next step utilizes sequence specific nuclease such as, for example, CRISPR enzyme guided to the target sequence(s) by one or more guide RNAs (gRNAs), followed by its nuclease activity as an actuator to allow convenient discrimination between specific and non-specific amplification. The isothermally amplified product is split into two equal volumes {A} and {B}. A master mix of (1) a sequence-specific ‘nuclease’ (e.g. Cas9) that induces double strand breaks (DSBs), (2) a ‘helicase’ (e.g. RepX) that evicts the ‘nuclease’ from dsDNA, and (3) a ‘double-stranded DNA exonuclease’ (e.g. lambda exonuclease) that efficiency degrades the amplified DNA from the cut site, are added to both {A} and {B}. In certain embodiments, aCas9 guide RNA with sequence complementary to the target of interest is used for {A}, while a non-targeting guide RNA is used for {B}. After room temperature or 37° C. incubation for up to 1 hour, DNA concentrations are measured for both {A} and {B}. On-target amplification in the initial isothermal amplification step results in Cas9 cleavage and exonuclease degradation of the on-target product. In contrast, off-target amplification product would not be cleaved and degraded. Therefore, a positive test is defined to be a significant, relative reduction in dsDNA concentration in volume {A} versus volume {B}. This can be conveniently determined using fluorescence measurements (from QuBit or Picogreen dye) on a plate reader. Anything else was deemed negative.

### A. 2× SENTINEL Buffer

(not active time—can be made in large batches for long-term −20° C. storage)

| Reagent | Volume | |
|---|---|---|
| NFW | 853 | μl |
| 2M Glycine in water | 67 | μl |
| 20 mg/ml BSA | 5 | μl |
| 100 mM ATP | 40 | μl |
| 1M MgCl2 | 25 | μl |
| 2M NaCl | 10 | μl |
| Total Volume | 1 | ml |

### B. RT-LAMP

| Reagent | Volume | |
|---|---|---|
| Nuclease free water | 2 | μl |
| Primer set | 1 | μl |
| LAMP 2× master mix | 5 | μl |
| Template (SARS-CoV-2) | 2 | μl |
| Total volume | 10 | μl |

a. Mix RT-LAMP reaction mixture;
b. Incubate 65° C. 30 min in thermocycler;
c. Dilute all 1:20 (add 190 μlto 10 μlrxn), mix, store in −20° C.

### C. Sentinel

| Reagent | Vol (1×) | |
|---|---|---|
| Nuclease free water | 6.44 | μl |
| 2× SENTINEL buffer | 10 | μl |
| 10 μM cr/trRNA | 0.2 | μl |
| 10 μM Cas9 | 0.16 | μl |
| SubTotal | 16.8 | μl |
| 1 μM RepX | 1 | μl |
| λ-exo (NEB) | 0.2 | μl |
| Total Volume (SENTINEL | 18 | μl |

master mix)

a. Mix in order up to SubTotal (18.36 μl);
b. Incubate room temperature for 30 min to form Cas9-gRNA complex (not active time—can be made in large batches for long-term −20° C. storage);
c. Add RepX and λ-exo to Total Volume (20 μl);
d. Mix 2 μl of diluted, unpurified RT-LAMP product with SENTINEL mixture;
e. Incubate room temperature for 20 min, measure DNA concentration with Picogreen on Plate reader.

### Example 2: Detection of Viral RNA

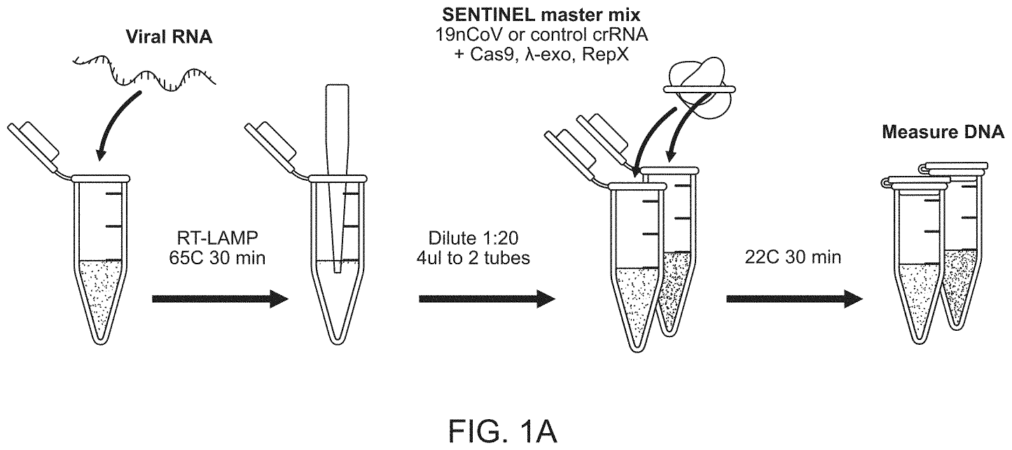

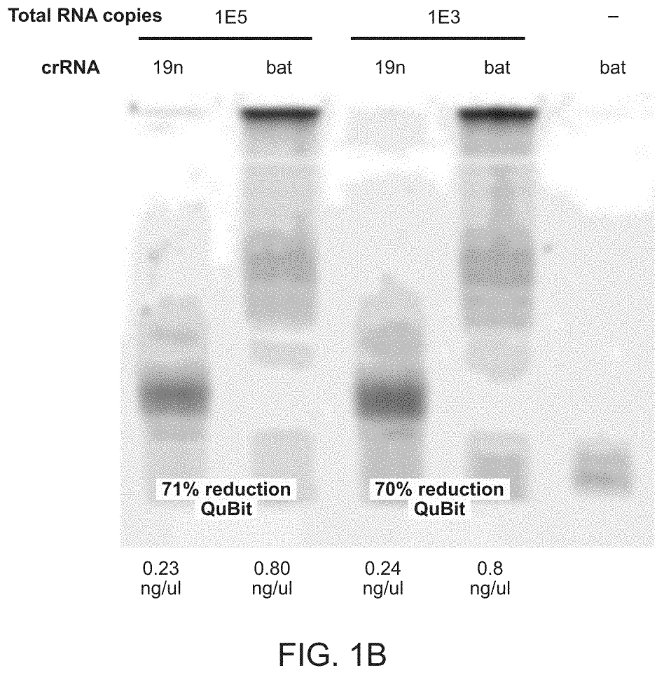

FIGS. 1A and 1B show the steps in the amplification and detection of the viral RNA. Input is synthetic viral RNA from SARS CoV2 (COVID 19). ‘19n’ corresponds to SARS CoV2 targeting guide RNA; ‘bat’ corresponds to non-targeting guide RNA (targets bat coronavirus); The concentration was measured concentration dsDNA specific dye (QuBit HS assay). A significant reduction in DNA concentration using targeting guide RNA (‘19n’) compared to non-targeting control (bat') was observed. The gel (FIG. 1B) shows degradation of RT LAMP product using ‘19n’, but not ‘bat’ guide RNA.

### Example 3: Diagnostics

DNA concentration was measured using microplate-based fluorescence readout of dsDNA-specific dye (Picogreen). Results are shown in Table 2.

| | | |
|---|---|---|
| Sample | Fluorescence (a.u.) | % Reduction |

| | | neg | 162 | |
|---|---|---|---|---|
| 1 × 10⁵ | copies | 19n | 7073 | 75.60 |
| | | bat | 28987 | |
| 1 × 10³ | copies | 19n | 7389 | 75.38 |
| | | bat | 30017 | |
| 1 | copy | 19n | 6948 | 74.73 |
| | | bat | 27494 | |
| 0 | copies | 19n | 2013 | 35.02 |
| | | bat | 3098 | |

1 × 10⁵ copies, 1 × 10³ copies, 1 copy, 0 copies correspond to number of viral RNA copies;
‘19n’ and ‘bat’ correspond to guide RNA targeting SARS-CoV2 or bat coronavirus, respectively;
‘neg’ corresponds to background fluorescence;
Use of ‘19n’ SARS-CoV2 targeting guide RNA greatly depleted DNA concentration;
Positive diagnostic test is determined by significant lower fluorescence using targeting vs non-targeting guide RNA.

### Example 4

Sequence-specific endonucleases such as restriction enzymes or RNA-guided endonucleases (i.e. Cas9) are highly efficient and specific. Cleavage generates two new free 5′ ends which can be targeted by lambda exonuclease (λ-exo) [8], a highly processive 5′ to 3′ exonuclease, for rapid degradation. The initial isothermal amplification (using primers resistant to exonuclease activity) and subsequent degradation after endonuclease cleavage are monitored using convenient fluorescence-based measurements of dsDNA concentration. Samples with the expected target would exhibit an amplification signal after LAMP and a loss of signal after endonuclease and λ-exo digestion. Samples with the incorrect target would either fail to amplify with LAMP, resulting in minimal initial signal, or generate a LAMP amplification product resistant to endonuclease and λ-exo treatment. We term this new rapid nucleic acid detection platform Specific Enzymatic Nucleic Acid Targeting with Nucleases and Exonuclease Lambda (SENTINEL). We demonstrate that this approach combines the attomolar-level sensitivity of isothermal amplification with the nucleotide-level specificity of sequence-specific endonucleases. The SENTINEL assay is highly convenient in requiring minimal laboratory reagents, modular in the choice of sequence-specific endonuclease, fast with under 1-hour total time and minimal hands-on time, and versatile in detecting SARS-CoV-2 virus particles in human saliva with high sensitivity.

SpCas9 purification. BL21-CodonPlus (DE3)-RIL competent cells (Agilent Technologies 230245) were transformed with Cas9 plasmid (Addgene #67881) and inoculated in 25 ml of LB-ampicillin media. The bacteria culture was first allowed to grow overnight (37° C., 220 rpm) and then transferred to 2 L of LB supplemented with ampicillin and 0.1% glucose until OD₆₀₀ of ˜0.5. Subsequently, the cells were induced with IPTG at a final concentration of 0.2 mM and maintained overnight at 18° C. The bacteria cells were pelleted at 4500×g, 4° C. for 15 min and resuspended in 40 ml of lysis buffer containing 20 mM Tris pH 8.0, 250 mM KCl, 20 mM imidazole, 10% glycerol, 1 mM TCEP, 1 mM PMSF, and cOmplete™ EDTA-free protease inhibitor tablet (Sigma-Aldrich 11836170001). This cell suspension was lysed using a microfluidizer and the supernatant containing Cas9 protein was clarified by spinning down cell debris at 16,000×g, 4° C. for 40 min and filtering with 0.2 μM syringe filters (Thermo Scientific™ F25006). 4 ml Ni-NTA agarose bead slurry (Qiagen 30210) was pre-equilibrated with lysis buffer. The clarified supernatant was then loaded at 4° C. The protein-bound Ni-NTA beads were washed with 40 ml wash buffer containing 20mM Tris pH 8.0, 800 mM KCl, 20 mM imidazole, 10% glycerol, and 1 mM TCEP. Gradient elution was performed with buffer containing 20 mM HEPES pH 8.0, 500 mM KCl, 10% glycerol, and varying concentrations of imidazole (100, 150, 200, and 250 mM) at 7 ml collection volume per fraction. The eluted fractions were tested on an SDS-PAGE gel and imaged by Coomassie blue (Bio-Rad 1610400) staining. To remove any DNA contamination, 5 mL HiTrap Q HP (Cytiva 17115401) was charged with 1 M KCl and then equilibrated with elution buffer containing 250 mM imidazole. The purified protein solution was then passed over the Q column at 4° C. The flow-through was collected and dialyzed in a 10 kDa SnakeSkin™ dialysis tubing (Thermo Fisher 68100) against 1 L of dialysis buffer (20 mM HEPES pH 7.5, 500 mM KCl, 20% glycerol) at 4° C., overnight. Next day, the protein was dialyzed for an additional 3 hours in fresh 1 L of dialysis buffer. The final Cas9 protein was concentrated to 10 μg/μL using Amicon Ultra-15 Centrifugal Filter Unit, Ultracel-10 (Millipore Sigma UFC901008), aliquoted, and flash-frozen and stored at −80° C.

eSpCas9 purification. The purification protocol was adapted from the manuscript associated with the eSpCas9 plasmid (Addgene #126769). Briefly, the eSpCas9 plasmid was transformed into BL21 Rosetta 2 (DE3) cells (Millipore EMD 71397) then grown in LB media with 10 μg/ml Kanamycin overnight at 37° C. 10 ml of this culture was inoculated into 1 L of LB media with 10 μg/ml Kanamycin and grown to a final cell density of 0.6 OD₆₀₀, then chilled at 18° C. The protein was expressed at 18° C. for 16 hours following induction with 0.2 mM IPTG. The cells were centrifuged at 6000×g for 15 min at 4° C., resuspended in 30 ml of Lysis Buffer (40 mM Tris pH 8.0, 500 mM NaCl, 20 mM imidazole, 1 mM TCEP) supplemented with cOmplete™ EDTA-free protease inhibitor tablet (1 tablet/30 ml; Sigma-Aldrich 11836170001), and then sonicated on ice. The lysate was cleared by centrifugation at 48,000×g for 40 min at 4° C., which was then bound to a 5 ml Mini Nuvia IMAC Ni-Charged column (Bio-Rad 7800812). The resin was washed extensively with a solution of 40 mM Tris pH 8.0, 500 mM NaCl, 20 mM imidazole, and the bound protein was eluted by a solution of 40 mM Tris pH 8.0, 250 mM imidazole, 150 mM NaCl, 1 mM TCEP. 10% glycerol was added to the eluted sample and the His6-MBP fusion protein was cleaved by TEV protease (Addgene pRK793) (3 h at 25° C.). The volume of the protein solution was made up to 100 ml with buffer (20 mM HEPES pH 7.5, 100 mM KCl, 1 mM DTT). The cleaved protein was purified on a 5 ml HiTrap SP HP cation exchange column (GE Healthcare 17115201) and eluted with 1 M KCl, 20 mM HEPES pH 7.5, 1 mM DTT. The protein was further purified by size exclusion chromatography on a HiPrep 26/60 Sephacryl S-200-HR column (GE 17 -1195-01) in 20 mM HEPES pH 7.5, 200 mM KCl, 1 mM DTT and 10% glycerol. The eluted protein was confirmed by SDS-PAGE and SimplyBlue™ SafeStain (Invitrogen LC6060). The protein was stored at −20° C.

Rep-X purification. E. coli Rep helicase was purified and crosslinked as previously described (Arslan et al., 2015). Briefly, the pET28(+) vector containing Rep-DM4 sequence was transformed into E. coli BL21(DE3) cells and grown in TB medium. When optical density reached OD600 =0.6 the protein overexpression was induced with 0.5 mM IPTG. The cells were incubated overnight at 18° C. and harvested by centrifugation at 10,000×g. Our Rep-DM4 contains 6×-His tag on its N-terminus for Ni-NTA affinity-column-based purification. The cell pellet was resuspended in a lysis buffer and cells were lysed with a sonicator. After binding Rep to Ni-NTA column and several washes, Rep was eluted with 150 mM imidazole-containing buffer. Rep concentration was kept below 4 mg/mL (approximately 50 μM) to avoid aggregation.

Our Rep-DM4 mutant has 4 native cysteines removed (C18L, C43S, C167V, C612A), while C178 is kept and S400C mutation is introduced for crosslinking. C178 and C400 are linked with bismaleimidoethane crosslinker (BMOE) to lock Rep in a closed conformation and form Rep-X. Optimal crosslinking is achieved at Rep concentration between 20 and 25 μM, with Rep to BMOE ratio of 1:5. Excess imidazole and crosslinker were removed by overnight dialysis in the storage buffer (50% glycerol, 600 mM NaCl, 50 mM Tris, pH 7.6). The samples are stored at −80° C. This method achieves nearly 95% crosslinking efficiency.

Generating synthetic ssRNA via in vitro transcription. For SARS-CoV-2 N-gene, perform PCR using 0.5 μL each of 10 μM forward and reverse primers (IVT19n N2 FWD, IVT19n N2 REV; primer sequences in Table S1). Mix with 4 μL of water, 0.5 μL of 2019-nCoV N Positive Control template plasmid (Integrated DNA technologies 10006625), and 10 μL of Q5 2× master mix (New England BioLabs M0494). Thermocycle at 98° C. for 30 sec for initial denaturation, followed by 35 cycles of 98° C. for 10 sec, 69° C. for 20 sec, 72° C. for 30 sec, then final extension of 72° C. for 2 min and 4° C. hold. Use QlAquick PCR purification kit (Qiagen 28104) to clean up PCR reaction and elute in 35 μl EB supplied with kit—agarose gel of PCR product should visualize band at 931 base pairs.

For in vitro transcription, use the HiScribe T7 kit (New England BioLabs E2050) by mixing 17 μl of purified PCR product with 3 μL of 10x reaction buffer, 8μL of NTP mix, 2.5 μl of 50mM DTT, and 2μL of T7 RNA polymerase mix for a 30 μl total volume. Incubate at 37° C. at least 2 hours. Then, add 20 μL of water and 1 μL of DNase I, mix, and incubate for another 37° C. for 15 min. Perform reaction cleanup using Monarch® RNA Cleanup Kit (New England BioLabs T2040). Measure RNA concentration using Nanodrop.

For bat-SL-CoVZC45 N-gene, perform the same protocol with IVT_bat_N2_FWD, IVT_bat_N2_REV primers (sequences in Table 3), with the SARS-CoV Control template plasmid (Integrated DNA technologies 10006624).

Making stock reagents (LAMP primer mix, cr/tracrRNA, buffers, and master mix) for CRISPR-based SENTINEL assay. Make 100 μl (RT)-LAMP primer mix by mixing 8 μl of FIP, 8 μl of BIP, 2 μl of F3, 2 μl of B3, 4 μl of LF, 4 μl of LB, and 56 μl of nuclease free water. All primers stocks are 100 μM in TE buffer, and sequences in Table 3.

Make stock 2× SENTINEL buffer by mixing 1-part 10× Lambda Exonuclease Reaction Buffer (New England BioLabs M0262), 1 part 100 mM NaCl, 1 part 100 mM MgCl₂, and 2-part 10 mM ATP, resulting in a solution composed of 67 mM Glycine-KOH, 2.5 mM MgCl₂, 50 μg/ml BSA, 2 mM ATP, 10 mM MgCl₂, and 10 mM NaCl.

To make 10 μM cr/tracrRNA, anneal 3 μl of crRNA with 3 μl of tracrRNA (Integrated DNA Technologies), both at stock concentrations of 100 μM in Duplex Buffer (Integrated DNA Technologies 11-01-03-01). Heat at 95° C. in a thermocycler with heated lid for 3 min, cool on benchtop for 5 min, then add 27 μl of Duplex Buffer to make 30 μl total.

To make 10 μM Cas9, mix 5 μl of 10 μg/μl Cas9 with 25 μl of Dialysis Buffer (20 mM HEPES pH 7.5, 500 mM KCl, 20% glycerol; this is the storage buffer used from Cas9 purification) to make 30 μl total.

To make 10 μM Cpf1, mix 5 μl of 10 μg/μl Alt-R®A.s. Cas12a (Cpf1) Ultra (Integrated DNA Technologies 10001272) with 25 μl of Dialysis Buffer from Cas9 purification to make 30 μl total (Cpf1 and Cas9 are approximately the same molecular weight).

To make Cas9-based 1× SENTINEL master mix for one reaction, mix 80 μl nuclease free water with 100 μl 2× SENTINEL buffer. Then, add 2 μl of 10 μM annealed cr/trRNA and 1.6 μl of 10 uM Cas9, then mix again. After room temperature incubation for 30 min to form the Cas9-gRNA complex, add 10 μl of 1 μM Rep-X, 2 μl of Lambda Exonuclease (New England BioLabs M0262), then mix again. This is sufficient for 10 reactions. This master mix can be made in larger batches, aliquoted to smaller volumes, and stored in −20° C. or −80° C.

To make Cpf1-based 1× SENTINEL master mix, replace Cas9 with 1.6 μl of 10 uM Cpf1. Rep-X is also not required and is omitted from the master mix.

For experiment with omission of Rep-X and/or Lambda Exonuclease, add equivalent volumes of water instead.

CRISPR-based SENTINEL assay on synthetic ssRNA or dsDNA. Mix 7 μL of nuclease free water, 2 μL of phosphorothiolated LAMP primer mix, and 10 μL of 2× LAMP master mix (New England BioLabs E1700) per reaction. Mix in 1 μL of diluted, synthetic ssRNA or dsDNA, then incubate at 65° C. for 30 min. Afterwards, add in 380 μl of TE buffer (1:20 dilution). Take 2 μL of this diluted (RT)-LAMP product, mix with 18 μl of 1× SENTINEL master mix, then leave at room temperature for 30 minutes. This is done twice—once with master mix containing on-target gRNA, and another with master mix containing non-target gRNA. Add in 180 μL of 1× PicoGreen solution (1 μL PicoGreen reagent with 200 μL TE buffer) (Thermo Fisher P7589), mix well, then load all to a well of Nunc™ F96 MicroWell™ Black Polystyrene Plate (Thermo Fisher 237105)

Using excitation wavelength of 485 nm and emission wavelength of 528 nm, measure the sample fluorescence using a Synergy H1 plate reader (BioTek).

For the separate negative control, (RT)-LAMP of input without spiked-in ssRNA/dsDNA was used in SENTINEL with the non-target gRNA.

Restriction enzyme (Afel) SENTINEL assay on synthetic ssRNA or dsDNA. Only the reaction master mix composition is modified from the CRISPR-based SENTINEL assay. Prepare reaction master mix by mixing 16 μL water, 2 μL CutSmart Buffer (New England BioLabs), 0.2 μL AfeI (New England BioLabs), and 0.2 μL Lambda Exonuclease (New

England BioLabs). This can be used to react with 2 μL of diluted (RT)-LAMP product for the assay.

For the non-targeting condition, Afel is replaced with equal volume of water. For the negative control, (RT)-LAMP of input without spiked-in ssRNA/dsDNA was used in SENTINEL where Afel is replaced with equal volume of water.

CRISPR-based SENTINEL assay on heat-inactivated viral particles. Gentamicin/Amphotericin B mixture was first made by mixing equal volumes of 50 mg/mL Gentamicin (Sigma-Aldrich G1397) with 250 μg/mL Amphotericin B (Sigma-Aldrich A2942), then filter-sterilized using a 0.22 μm pore size filter unit. Viral transport media (VTM) was prepared by mixing 500 mL of Hanks Balanced Salt Solution (HBSS), 10 mL heat-inactivated FBS (Corning), and 2 mL of filter-sterilized Gentamicin/Amphotericin B mixture.

Supernatant from SARS-CoV-2 infected Vero cell culture with 10E7 viral particles per μL was heated at 65° C. for 1 hour for virus inactivation. Serial dilutions were performed using VTM. 1 μL of the dilution was mixed with 4 μL Pooled Human Saliva (Innovative

Research IRHUSL5ML) to simulate virus presence in human saliva. This was mixed 1:1 with QuickExtract™ DNA Extraction Solution (Lucigen QE09050), then immediately heated to 95° C. for 5 min. 1μL of this final solution was used in a 20 μL SENTINEL reaction, with the remainder of the protocol identical to the section “CRISPR-based SENTINEL assay on synthetic ssRNA or dsDNA”.

Computation of SENTINEL score. Reaction A corresponds to the on-target SENTINEL reaction (on-target Cas/gRNA or on-target restriction enzyme). Reaction B corresponds to the non-target SENTINEL reaction (non-target Cas9/gRNA or water in the place of restriction enzyme). Reaction C corresponds to the negative control—the sample expected to have no target nucleic acid, exposed to the non-target SENTINEL reaction. A, B, and C will be the results of Reactions A, B, and C, respectively, as fluorescence measurements (in arbitrary units) on a plate reader.

The first part of the SENTINEL score is fractional reduction in fluorescence with on-target versus non-target Cas9/gRNA, i.e., 1-AB. Next, this value is scaled by the fractional increase in DNA quantity due to LAMP amplification (B/C). Together, the SENTINEL score is computed using the following formula: (1-AB) * (B/C).

### Results

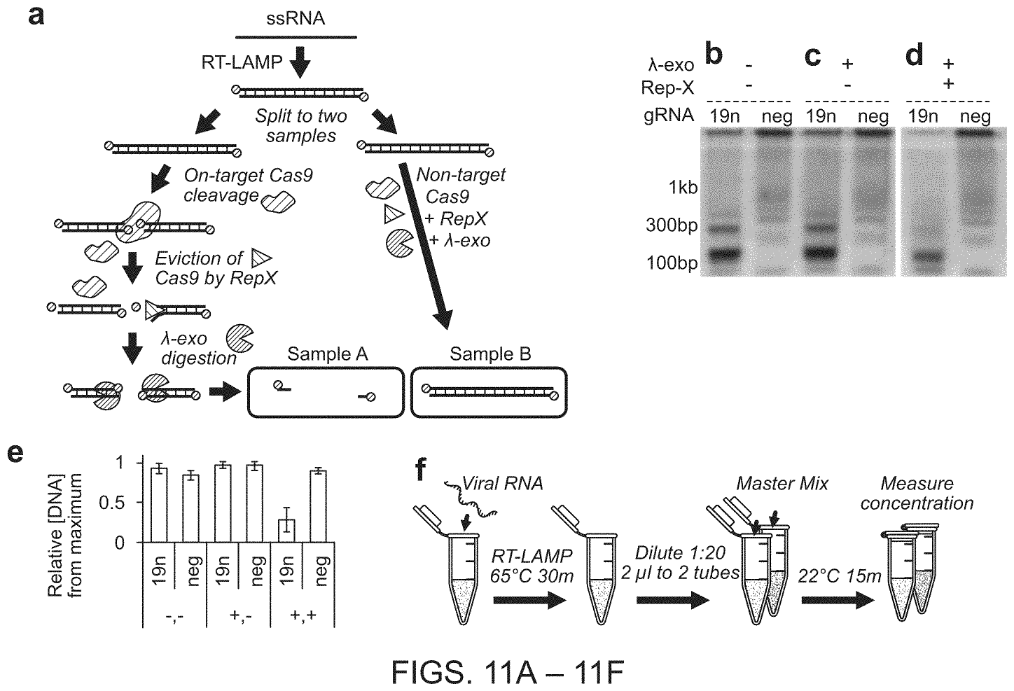

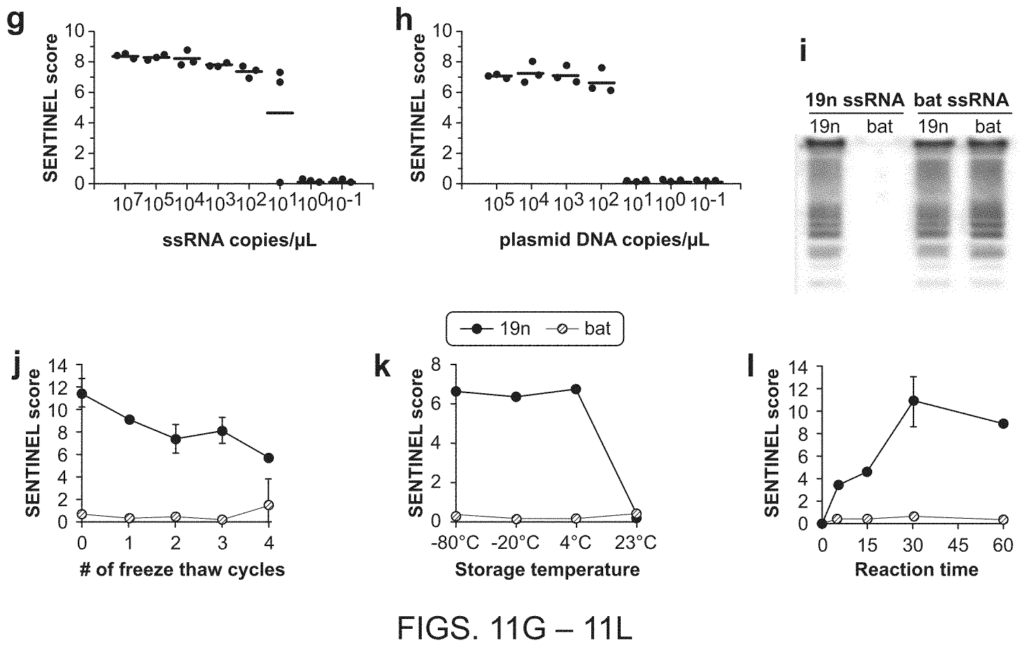

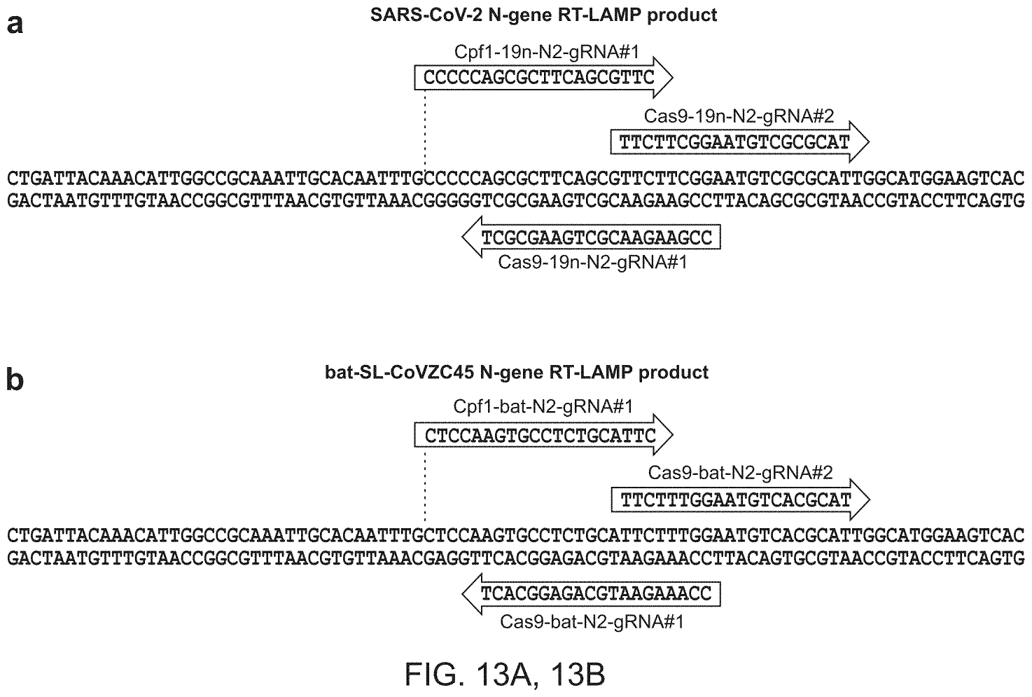

For initial validation, we evaluated wild type SpCas9 as the endonuclease [9]. Using in vitro transcription, we generated synthetic RNA from the N-gene of SARS-CoV-2 as the target of interest [10] (FIGS. 11A; 13A). Reverse transcription LAMP (RT-LAMP) was first performed at 65° C. for 30 minutes. The RT-LAMP primers were designed to all contain 5′ phosphorothiolate modifications, resulting in product DNA that resists λ-exo degradation. We first verified that Cas9 can efficiently cleave RT-LAMP product by mixing diluted RT-LAMP product with Cas9 in complex with crRNA and tracrRNA (henceforth referred to as gRNA) targeting the SARS-CoV-2 N-gene (Cas9-19n-N2-gRNA#1) (FIGS. 13A, 13B), or a non-targeting gRNA. We observed a band shift in RT-LAMP product on agarose gel with use of Cas9/gRNA targeting the SARS-CoV-2 sequence (‘19n’) compared to use of non-targeting Cas9/gRNA (‘neg’), suggesting efficient Cas9 cleavage (FIG. 11B). However, addition of λ-exo to the reaction unexpectedly did not lead to degradation of cleavage product (FIG. 11C). We hypothesized that λ-exo loading was inhibited by Cas9 molecules remaining on the target DNA after cleavage. To overcome this obstacle, we hypothesized that a highly processive helicase, such as the recently engineered Rep-X “super”-helicase [11], could be used to evict Cas9. As expected, we found that addition of Rep-X and ATP to the λ-exo reaction greatly increased digestion (‘19n’) (FIGS. 11D-11E). Cas9 combined with a non-target gRNA ('leg') resulted in no detectable degradation upon addition of Rep-X and/or λ-exo, consistent with Cas9 cleavage being the actuator for DNA degradation viaλ-exo (FIGS. 11A-11E).

To greatly simplify the diagnostic assay, we created a “master mix” composed of Glycine-KOH buffer with preformed SpCas9-gRNA complex, λ-exo, Rep-X, ATP, and MgCl₂. Starting from RNA as the sample of interest, RT-LAMP was first employed for isothermal amplification at 65° C. for 30 minutes on a heat block (alternatively using LAMP for DNA samples of interest) (FIG. 11F). 2 μL of 1:20-diluted RT-LAMP product was incubated with 18 μL of the SENTINEL master mix in two tubes, one composed of on-target (Reaction A) and the other of non-target Cas9/gRNA (Reaction B), for 15-30 minutes at room temperature. For a negative control reference to account for variation between reagent batches, RT-LAMP was also performed without any target nucleic acid, followed by the SENTINEL reaction using non-target Cas9/gRNA (Reaction C). Relative DNA concentrations from Reactions A, B, and C were conveniently measured using a fluorescent plate reader after 1:10 dilution with 1× PicoGreen dye, a dsDNA-specific fluorescent dye that exhibits over 1000-fold increase in fluorescence upon dsDNA intercalation [12].

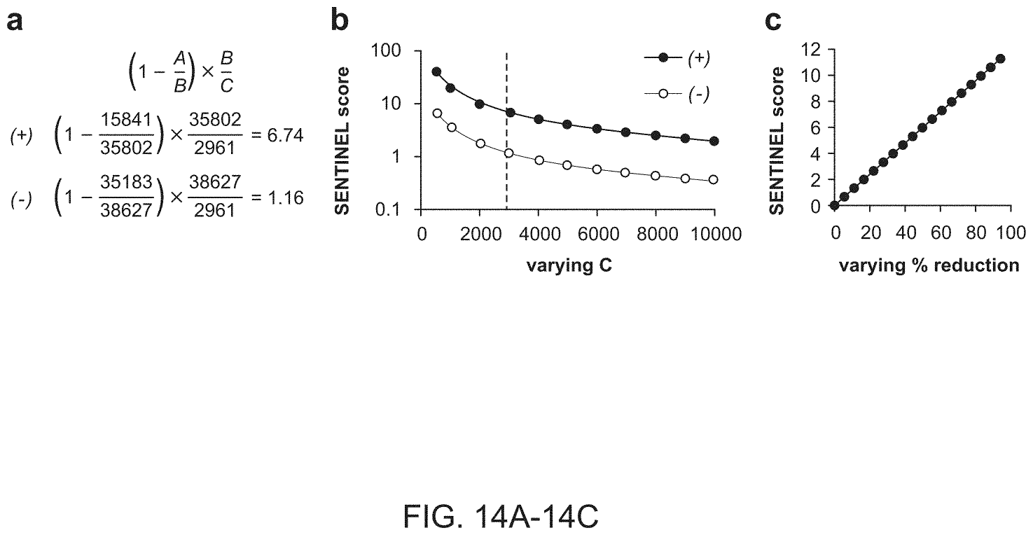

We aimed to summarize the three fluorescence measurements (A, B, C) as a single numerical score, where a high score would indicate detection of the target nucleic acid, i.e., a positive test. First, we calculated the fractional increase in LAMP product due to the presence of target sample (B/C), since samples can only have a positive test if LAMP successfully amplified. Second, we determined the fractional reduction in dsDNA fluorescence of LAMP product with use of target versus non-targeting Cas9/gRNA (1-A/B). If the target product was amplified by LAMP, target Cas9/gRNA should efficiently cleave to allow λ-exo degradation, resulting in a larger reduction. In contrast, if undesired, off-target product was amplified by LAMP, then use of target Cas9 would have minimal effect relative to a non-targeting Cas9/gRNA, resulting in no reduction. Both quantities, (1-A/B) and (B/C), should have large values to have a positive test, motivating the final SENTINEL score to be computed as (1-A/B) * (B/C). The SENTINEL score is high if LAMP results in amplification products and if these products are degraded by the nucleases, and is low if either condition is not met (FIG. 14A). Perturbation analysis varying either C or A relative to B (% reduction) illustrates how the score's value may vary, but the difference in scores between positive and negative samples remains separable and directly related to the extent of their respective λ-exo mediated degradation (FIGS. 14B, 14C).

With the assay and its performance metric at hand, we evaluated the compatibility of the assay for different input types, as well as its limit of detection. Using SARS-CoV-2 in vitro transcribed ssRNA or a (dsDNA) plasmid encoding the N-gene, we performed (RT)-LAMP using 5′ phosphorothiolated LAMP primers, followed by incubation with SENTINEL master mix containing either on-target gRNA targeting the SARS-CoV-2 template sequence or a non-targeting gRNA. We evaluated the capability of the assay to detect varying concentrations of template though sequential serial dilutions. First, we determined that the master mix was functional—with appropriate concentrations of template, the assay resulted in SENTINEL scores of over 6, versus a score that approaches 0 when no template was supplied or template quantity under the limit of detection (FIGS. 11G, 11H). Furthermore, we found that this strategy could reliably detect down to approximately 100 ssRNA or dsDNA molecules per microliter of input sample, which is in the attomolar concentration range. The sensitivity of this assay is bounded by the sensitivity of the LAMP step, which has been shown to be comparable to gold standard quantitative PCR (qPCR) [13]. Together, these results demonstrate that our assay exhibits attomolar levels of sensitivity with clear discrimination between positive and negative test results.

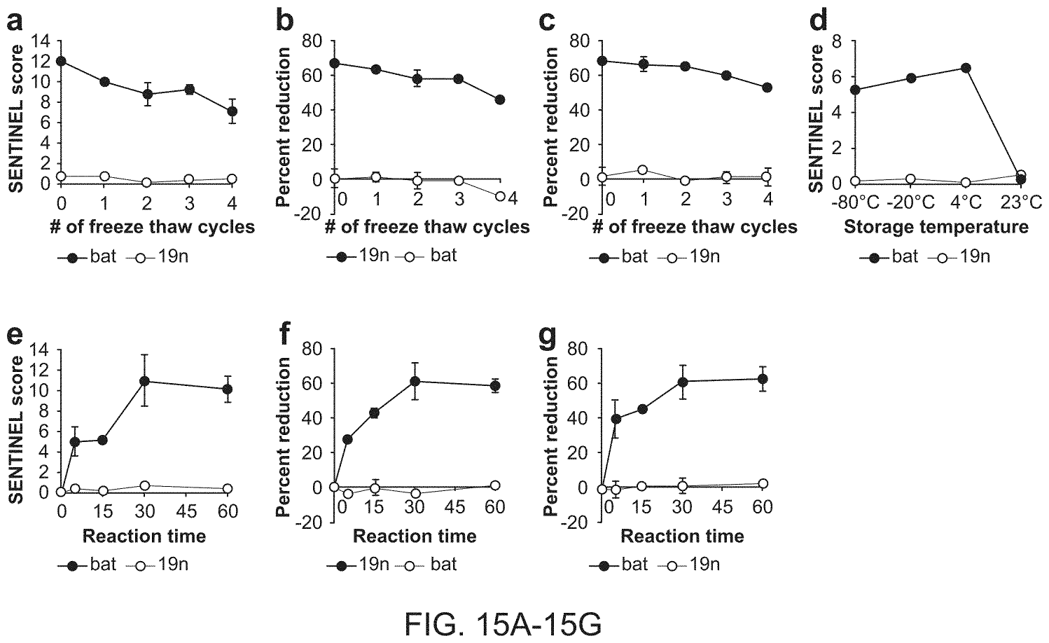

Next, we evaluated the assay's ability to distinguish between closely related target sequences, the stability of its reagents, and its required reaction duration. We performed the assay on in vitro transcribed synthetic RNA encoding either SARS-CoV-2 (19nCoV) or the closely related bat-SL-CoVZC45 (batCoV) [14] N-gene. For the SENTINEL reaction, they were paired with Cas9 gRNAs targeting either 19nCoV (Cas9-19n-N2-gRNA#1) or batCoV (Cas9-bat-N2-gRNA#1), whose sequences differed by 5 nucleotides (FIGS. 13A, 13B). Notably, use of RT-LAMP alone for 19nCoV detection resulted in a false positive readout when batCoV ssRNA was in the sample solution (FIG. 11I). In comparison, our method did not have a false positive readout—correct pairings of RT-LAMP product with gRNA resulted in a score of over 6, while incorrect pairings (i.e., 19nCoV gRNA with batCoV RT-LAMP product) resulted in a SENTINEL score of under 2 (FIGS. 11J, 15A). We verified that this difference was due to large, expected reductions in DNA concentrations with use of the on-target versus non-targeting gRNA, as measured by PicoGreen fluorescence (FIGS. 15B, 15C). In addition, the master mix was stable for multiple −80° C. to room temperature freeze-thaw cycles (FIGS. 11J; 15A), as well as long-term storage up to 1 month in −80° C., −20° C., and 4° C., but not room temperature (FIGS. 11K; 15D). Next, by performing the endonuclease reaction as a function of time, we found that even 5 minutes was sufficient to achieve clear discrimination between expected positive and negative samples (FIGS. 11L, 15E-15G).

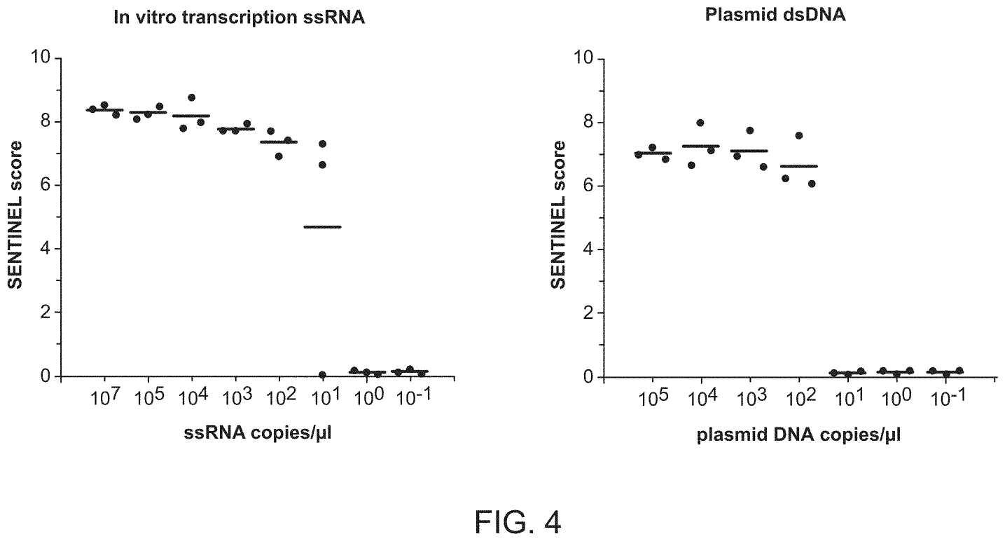

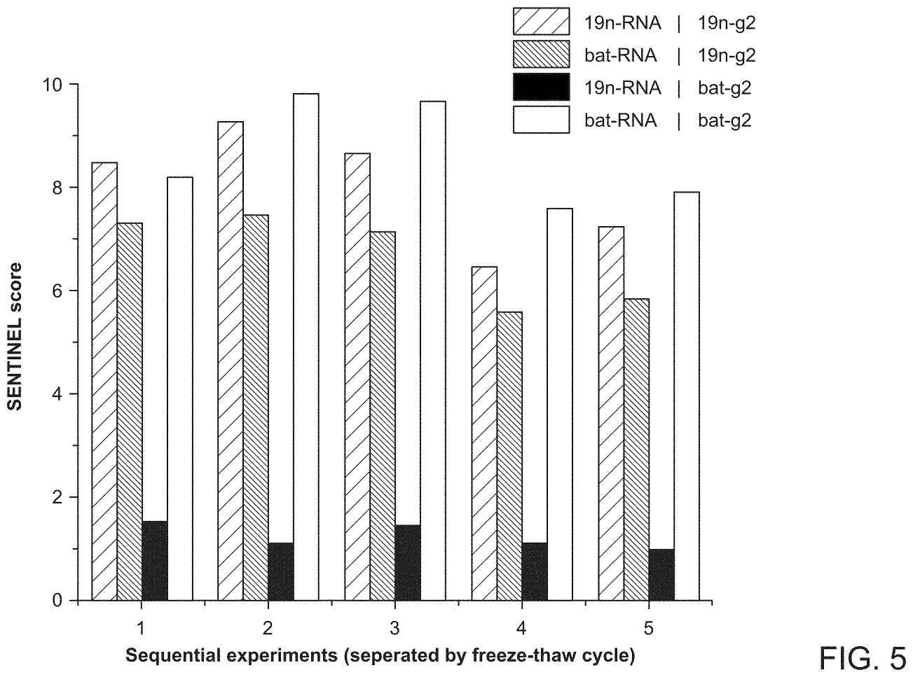

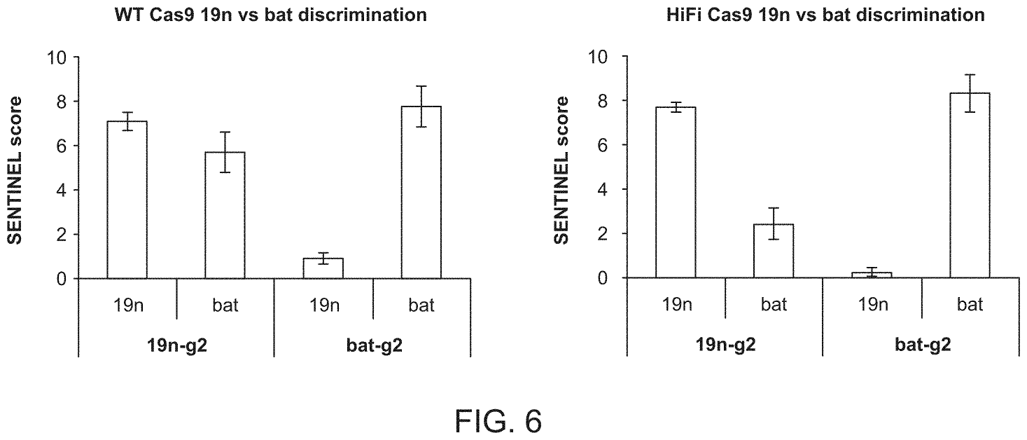

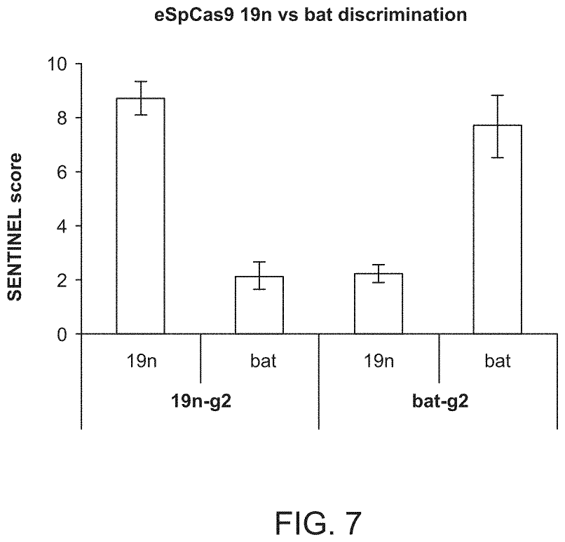

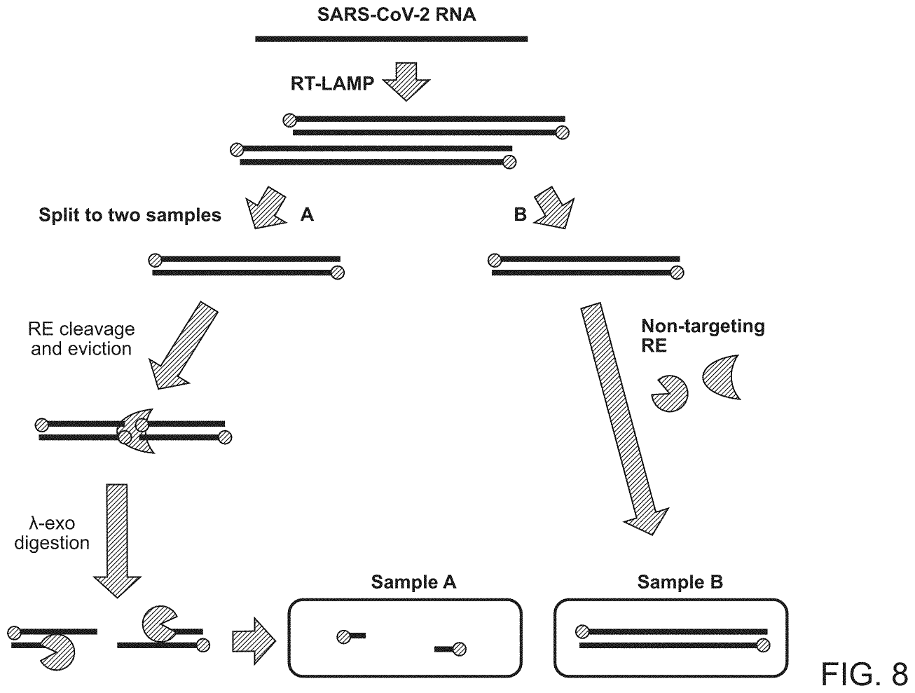

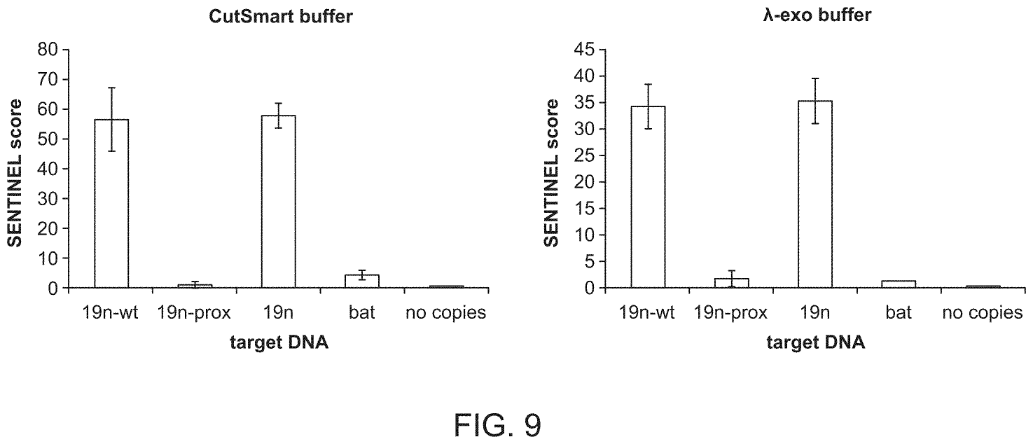

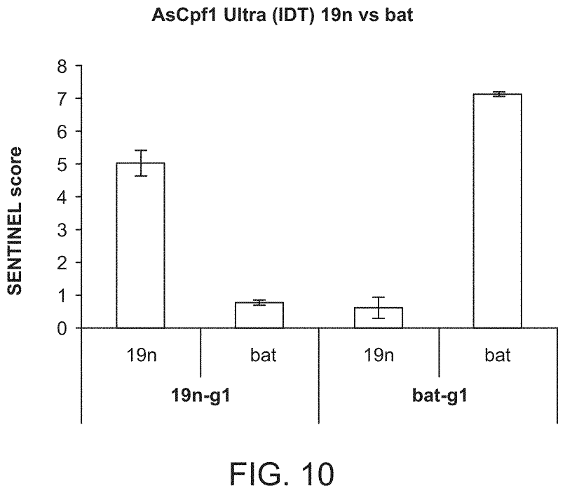

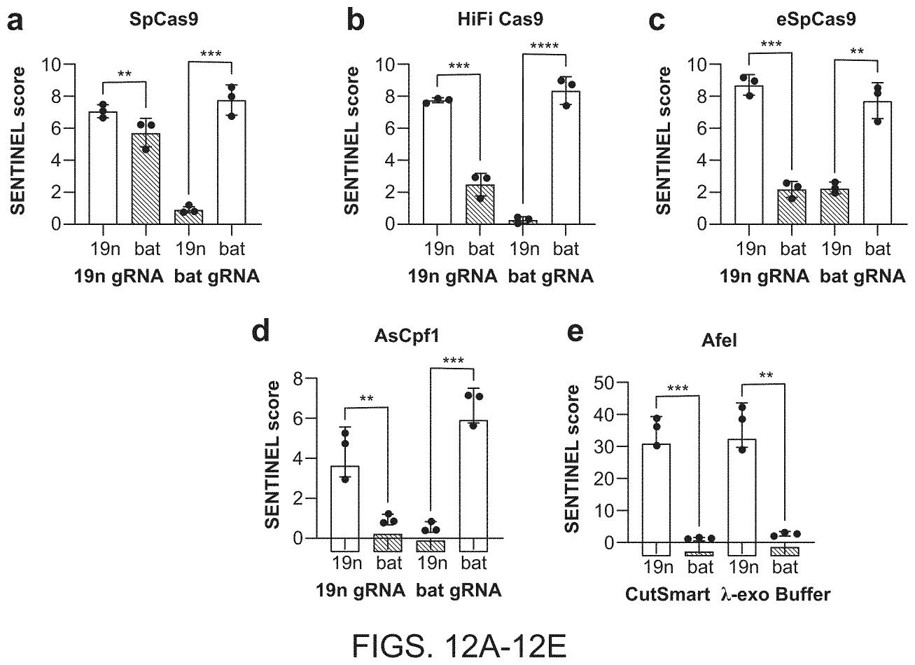

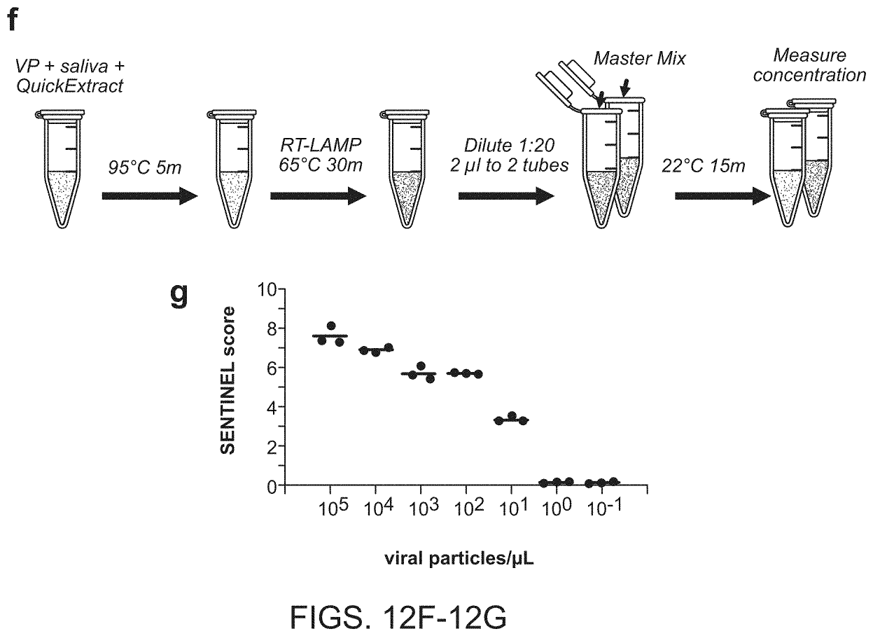

Because SENTINEL relies on targeted endonuclease cleavage to actuate exonuclease-driven target discrimination, we evaluated whether other CRISPR endonucleases such as enhanced-specificity Cas9s and Cas12a [15] would be compatible. To better evaluate the specificity advantages of engineered Cas9s in the SENTINEL system, we designed new gRNAs targeting the N-gene of SARS-CoV-2 (Cas9-19n-N2-gRNA#2) and bat-SL-CoVZC45 (Cas9-19n-N2-gRNA#2) that differed by only two nucleotides in the protospacer (FIGS. 11A-11I). Use of wild type Cas9 for 19nCoV detection was notable for a false positive readout when batCoV ssRNA was in the sample solution (FIG. 12A). In contrast, use of enhanced specificity HiFi Cas9 and eSpCas9 [17,18] resulted in a much lower SENTINEL score for incorrect pairings of input nucleic acids and gRNA, consistent with better discrimination of two-nucleotide mismatches using enhanced specificity Cas9 systems (FIGS. 12A-12C). We also determined that use of Cas12a (AsCpf1) with gRNAs targeting either the 19nCoV (Cpf1-19n-N2-gRNA#1) or batCoV (Cpf1-bat-N2-gRNA#1) N-gene, which differed by 5 nucleotides in the protospacer (FIG. 5I), resulted in assay performance comparable to that of Cas9 (FIG. 12D). Notably, Rep-X and ATP were dispensable for the Cas12a reaction, consistent with automatic Cas12a departure from target DNA after cleavage [19], which would facilitate λ-exo loading and target DNA degradation without need for Rep-X.

We also tested the capability of restriction enzymes [16] to act as the sequence-specific endonuclease to actuate λ-exo cleavage. We identified the 6-cutter restriction enzyme Afel to have one target site in the 19nCoV RT-LAMP product, but not but not in the batCoV product due to a single mismatch. We replaced the previous Cas9/gRNA and Rep-X components with Afel, then performed SENTINEL (30 minutes at room temperature) on the RT-LAMP products of samples containing either 19nCoV or batCoV ssRNA. Because the majority of restriction enzymes are known to be functional in the acetate-based CutSmart Buffer, we evaluated the reaction using that buffer as well as the previous Glycine-KOH based λ-exo Buffer. For both CutSmart and λ-exo Buffer, we verified positive signal only for the 19nCoV sample, but not for the batCoV sample (FIG. 12E). Together, these results demonstrate that the assay has a high degree of flexibility for endonuclease selection and high specificity.

Finally, we determined the ability of this assay to detect SARS-CoV-2 virus particles in human saliva. We obtained heat-inactivated SARS-CoV-2 virus originating from the culture medium of SARS-CoV-2 infected VeroTMPRSS2 cells [20]. To mimic detection of viral particles in patient samples, we diluted virus titer in viral transport media (VTM), followed by 1:4 addition into mixed human saliva. To lyse the virus and extract its RNA, we adapted a previous protocol [21] by adding 1:1 of QuickExtract lysis buffer, followed by heating to 95° C. for 5 min. 1 μL of lysed viral particles was used for RT-LAMP and subsequent steps of the SENTINEL assay (FIG. 12F). By evaluating serial dilutions of the virus titer in saliva, we observed high sensitivity, consistently detecting down to 10 particles per microliter of the original input (FIG. 12G).

SENTINEL is a first-in-class method for nucleic acid detection that utilizes the target cleavage properties of sequence-specific endonucleases to verify sequence identity. Our approach is generalizable, in that any sequence-specific endonuclease can be utilized, and orthogonal to existing technologies by using reagents, such as lambda exonuclease and Rep-X, not previously used for nucleic acid detection. These features also allow SENTINEL to be rapidly deployed to meet the demands of diagnostic nucleic acid testing, which, as demonstrated by the recent COVID-19 pandemic, is greatly expedited by the availability of diverse assay methodologies. Furthermore, the limited number of liquid-handling steps and use of plate readers for readout allow SENTINEL to be amenable to 96/384-well plate formats and automation using liquid-handling robots for high-throughput sample processing. Future directions include extension to other formats such as portable diagnostic devices and use of patient-derived samples to determine its sensitivity and specificity as a clinical-grade diagnostic test for COVID-19.

Other CRISPR-based methods using Cas12 (DETECTR) and Cas13 (SHERLOCK) exhibited comparable features to our strategy [5,6]. The original SHERLOCK and DETECTR assays also require at least one heating temperature (37° C. or 65° C.) for isothermal amplification before CRISPR-based detection, and require approximately 1 hour to complete the entire protocol. Recently, both strategies have been adapted for SARS-CoV-2 detection [21,22]. The recent SHERLOCK assay variant for SARS-CoV-2 detection, STOPCovid.v2, simplified the protocol by designing a one-pot reaction that only requires one heating temperature. Compared to DETECTR for SARS-CoV-2, our strategy requires only 1 versus 2 different heating temperatures (because our second step is performed at room temperature whereas DETECTR's second step is performed at 37° C.). Because DETECTR requires Cas12, only sequence regions adjacent to a Cas12-specific protospacer adjacent motif (PAM) can be evaluated, whereas the flexible choice of endonuclease with SENTINEL allows evaluation of regions next to PAMs of other CRISPR enzymes, or potentially even without PAM restrictions using near-PAMless Cas9 variants [23].

In conclusion, SENTINEL expands the scope of nucleic acid detection by combining the convenience of isothermal amplification with improved specificity to discriminate between one or two base pair differences. It extends nucleic acid detection by utilizing the on-target cleavage properties, rather than bystander collateral cleavage, of CRISPR enzymes to enable use by diverse CRISPR systems from enhanced-specificity Cas9 to Cas12a, as well as other endonucleases such as restriction enzymes. The entire protocol can be completed in under 1 hour and only requires a 65° C. heat block and fluorometer on top of basic laboratory materials. The assay master mix is stable to multiple freeze-thaw cycles and long-term storage. Finally, SENTINEL can detect SARS-CoV-2 viral particles in human saliva with high sensitivity.

TABLE 3
Oligonucleotide sequences

| Name | Sequence |
|---|---|
| IVT_19n_N2_FWD | AATTCTAATACGACTCACTATAGGGCCAAATTGGCTACTACCGAAGAGCTAC |
| | (SEQ ID NO: 1) |
| IVT_bat_N2_FWD | AATTCTAATACGACTCACTATAGGGCCAAATTGGCTACTACCGTAGAGCTAC |
| | (SEQ ID NO: 2) |
| IVT_19n_N2_REV | CACAGTTTGCTGTTTCTTCTGTCTCTG (SEQ ID NO: 3) |
| IVT_bat_N2_REV | CACAGTTTGTTGTTTCTTCTGTCTCTGC (SEQ ID NO: 4) |
| RTL_19n/bat_N2_FIP-p | T*G*C*G*G*CCAATGTTTGTAATCAGCCAAGGAAATTTTGGGGAC (SEQ ID |
| | NO: 5) |
| RTL_19n_N2_BIP-p | C*G*C*A*T*TGGCATGGAAGTCACTTTGATGGCACCTGTGTAG (SEQ ID NO: |
| | 6) |
| RTL_bat_N2_BIP-p | C*G*C*A*T*TGGCATGGAAGTCACtttaatggctccatgataa (SEQ ID NO: 7) |
| RTL_19n_N2_F3-p | A*A*C*A*C*AAGCTTTCGGCAG (SEQ ID NO: 8) |
| RTL_bat_N2_F3-p | c*a*c*t*c*aagcatttgggag (SEQ ID NO: 9) |
| RTL_19n_N2_B3-p | G*A*A*A*T*TTGGATCTTTGTCATCC (SEQ ID NO: 10) |
| RTL_bat_N2_B3-p | g*a*a*t*t*gtggatctttgtcatcc (SEQ ID NO: 11) |
| RTL_19n_N2_LF-p | T*T*C*C*T*TGTCTGATTAGTTC (SEQ ID NO: 12) |
| RTL_bat_N2_LF-p | t*t*c*c*t*tgtctgattaattc (SEQ ID NO: 13) |
| RTL_19n_N2_LB-p | A*C*C*T*T*CGGGAACGTGGTT (SEQ ID NO: 14) |
| RTL_bat_N2_LB-p | a*c*c*t*t*cgggaacatggct (SEQ ID NO: 15) |
| RTL_19n_N2_F3-p | A*A*C*A*C*AAGCTTTCGGCAG (SEQ ID NO: 16) |
| RTL_bat_N2_F3-p | c*a*c*t*c*aagcatttgggag (SEQ ID NO: 17) |

-Asterisk (*) corresponds to phosphorothiolate modifications

TABLE 4
guide RNA protospacer sequences

| Name | Protospacer sequence |
|---|---|
| Cas9-19n-N2-gRNA#1 | CCGAAGAACGCTGAAGCGCT |
| | (SEQ ID NO: 18) |
| Cas9-bat-N2-gRNA#1 | CCAAAGAATGCAGAGGCACT |
| | (SEQ ID NO: 19) |
| Cas9-19n-N2-gRNA#2 | TTCTTCGGAATGTCGCGCAT |
| | (SEQ ID NO: 20) |
| Cas9-bat-N2-gRNA#2 | TTCTTTGGAATGTCACGCAT |
| | (SEQ ID NO: 21) |
| Cpf1-19n-N2-gRNA#1 | CCCCCAGCGCTTCAGCGTTC |
| | (SEQ ID NO: 22) |
| Cpf1-bat-N2-gRNA#1 | Ctccaagtgcctctgcattc |
| | (SEQ ID NO: 23) |
| Cpf1-negctrl-gRNA#1 | CGTTAATCGCGTATAATACGG |
| | (SEQ ID NO: 24) |

*Cas9 negative control is commercially purchased as Alt-R ® CRISPR-Cas9 Negative Control crRNA #1 (Integrated DNA Technologies 1072544)

### REFERENCES FOR EXAMPLE 4

1. Urdea, M.; Penny, L. A.; Olmsted, S. S.; Giovanni, M. Y.; et al. Requirements for high impact diagnostics in the developing world. Nature 2006, 444 (1), 73-79.
2. Pardee, K.; Green, A. A.; Takahashi, M. K.; Braff, D.; et al. Cell 2016, 165 (5), 1255-1266.
3. Esbin, M. N.; Whitney, O. N.; Chong, S.; Maurer, A.; et al. Overcoming the bottleneck to widespread testing: a rapid review of nucleic acid testing approaches for COVID-19 detection. RNA 2020, 26 (7), 771-783.
4. Bustin, S. A.; Nolan, T. RT-qPCR testing of SARS-CoV-2: a primer. Int. J. of Mol. Sci. 2020, 21 (8), 3004.
5. Notomi, T.; Okayama, H.; Masubuchi, H.; Yonekawa, T.; et al. Loop-mediated isothermal amplification of DNA. Nucleic Acids Res. 2000, 28 (12), 63.
6. Gootenberg, J. S.; Abudayyeh, O.O .; Lee, J.W.; Essletzbichler, P.; et al. Nucleic acid detection with CRISPR-Cas13a/C2c2. Science 2017, 356 (6336), 438-442.
7. Chen, J. S.; Ma, E.; Harrington, L. B.; Da Costa, M.; et al. CRISPR-Cas12a target binding unleashes indiscriminate single-stranded DNase activity. Science 2018, 360 (6387), 436-439.
8. Lee, G.; Yoo, J.; Leslie, B. J.; Ha, T. Single-molecule analysis reveals three phases of DNA degradation by an exonuclease. Nat. Chem. Bio. 2011, 7 (6), 367-374.
9. Jinek, M.; Chylinski, K.; Fonfara, I.; Hauer, M.; et al. A programmable dual-RNA—guided DNA endonuclease in adaptive bacterial immunity. Science 2012, 337 (6096), 816-821.
10. Andersen, K. G.; Rambaut, A.; Lipkin, W. I.; Holmes, E. C.; Garry, R. F. The proximal origin of SARS-CoV-2. Nat. Med. 2020, 26 (4), 450-452.
11. Arslan, S., Khafizov, R., Thomas, C. D., Chemla, Y.R. and Ha, T., 2015. Engineering of a superhelicase through conformational control. Science 2015, 348 (6232), 344-347.
12. Dragan, A. I.; Casas-Finet, J. R.; Bishop, E. S.; Strouse, R. J.; et al. Characterization of PicoGreen interaction with dsDNA and the origin of its fluorescence enhancement upon binding. Biophys. 1 2020, 99 (9), 3010-3019.
13. Khan, M.; Wang, R.; Li, B.; Liu, P.; et al. Comparative evaluation of the LAMP assay and PCR-based assays for the rapid detection of Alternaria solani. Front. Microbiol. 2018, 9, 2089.
14. Lu, R.; Zhao, X.; Li, J.; Niu, P.; et al. Genomic characterisation and epidemiology of 2019 novel coronavirus: implications for virus origins and receptor binding. Lancet 2020, 395 (10224), 565-574.
15. Pickar-Oliver, A.; Gersbach, C. A. The next generation of CRISPR—Cas technologies and applications. Nat. Rev. Mol. Cell Biol. 2019, 20 (8), 490-507.
16. Roberts, R. J. How restriction enzymes became the workhorses of molecular biology. Proc. Natl. Acad. Sci. U S. A. 2005, 102 (17), 5905-5908.
17. Vakulskas, C. A.; Dever, D. P.; Rettig, G. R.; Turk, R.; et al. A high-fidelity Cas9 mutant delivered as a ribonucleoprotein complex enables efficient gene editing in human hematopoietic stem and progenitor cells. Nat. Med. 2018, 24 (8), 1216-1224.
18. Slaymaker, I. M.; Gao, L.; Zetsche, B.; Scott, D.A.; Yan, W.X.; Zhang, F. Rationally engineered Cas9 nucleases with improved specificity. Science 2016, 351 (6268), 84-88.
19. Singh, D.; Mallon, J.; Poddar, A.; Wang, Y.; et al. Real-time observation of DNA target interrogation and product release by the RNA-guided endonuclease CRISPR Cpfl (Cas12a). Proc. Natl. Acad. Sci. U. S. A. 2018, 115 (21), 5444-5449.
20. Joung, J.; Ladha, A.; Saito, M.; Kim, N.G.; et al. Detection of SARS-CoV-2 with SHERLOCK one-pot testing. N. Engl. J. Med. 2020, 383 (15), 1492-1494.
21. Klein, S. L.; Pekosz, A.; Park, H. S.; Ursin, R. L.; et al. Sex, age, and hospitalization drive antibody responses in a COVID-19 convalescent plasma donor population. J. Clin. l Investig. 2020, 130 (11), 6141-6150.
22. Broughton, J. P.; Deng, X.; Yu, G.; Fasching, C. L.; et al. CRISPR—Cas12-based detection of SARS-CoV-2. Nat. Biotechnol. 2020, 38 (7), 870-874.
23. Walton, R. T.; Christie, K. A.; Whittaker, M. N.; Kleinstiver, B. P. Unconstrained genome targeting with near-PAMless engineered CRISPR-Cas9 variants. Science 2020, 368 (6488), 290-296.

### OTHER EMBODIMENTS

From the foregoing description, it will be apparent that variations and modifications may be made to the invention described herein to adopt it to various usages and conditions. Such embodiments are also within the scope of the following claims.

All citations to sequences, patents and publications in this specification are herein incorporated by reference to the same extent as if each independent patent and publication was specifically and individually indicated to be incorporated by reference. By their citation of various references in this document, applicants do not admit any particular reference is “prior art” to their invention.

1. A method of detecting a nucleic acid of interest in a sample, comprising:
preparing primers complementary to the nucleic acid of interest wherein the primers are protected against nuclease activity;
performing isothermal amplification to amplify small amounts of either RNA or DNA sequences from the sample to double stranded DNA (dsDNA);
subjecting the amplified nucleic acid of interest to a sequence-specific nuclease; and,
detecting the nucleic acid of interest.

2. The method of claim 1, wherein the nuclease activity is inhibited by exonuclease inhibitors comprising: citrate, citrate acid; MES, 2-morpholin-4-ylethanesulfonate; PV6R, pontacyl violet 6R; PCMPS, p-chloromercuriphenyl sulfonate; NCA, 7-nitroindole-2-carboxylic acid; DR396, 4-[(4,6-dichloro-1,3,5-triazin-2-yl)amino]-2-(3-hydroxy-6-oxoxanthen-9-yl)benzoic acid; DTNB, 5,5′-dithiobis(2-nitrobenzoic acid); ATA, aurintricarboxylic acid; FDCO, fmoc-D-Cha-OH, Mirin, PFM01/SML1735, PFM03, PFM39/SML1839 or combinations thereof.

3. The method of claim 1, wherein the primers comprise phosphorothiolate bonds, secondary structures, polylinkers, fluorescent tags, biotin, affinity labels, reactive groups, 2′-O-modified riboses, inverted dT, inverted, 2′,3′ dideoxy-dT base (5′ Inverted ddT), phosphorylation, phosphoramidite C3 Spacer or combinations thereof

4. The method of claim 1, wherein the nuclease activity is inhibited by nuclease inhibitors comprising: diethyl pyrocarbonate, ethanol, aurintricarboxylic acid (ATA), formamide, vanadyl-ribonucleoside complexes, macaloid, ethylenediamine tetraacetic acid (EDTA), proteinase K, heparin, hydroxylamine-oxygen-cupric ion, bentonite, ammonium sulfate, dithiothreitol (DTT), beta-mercaptoethanol, cysteine, dithioerythritol, tris (2-carboxyethyl) phosphene hydrochloride, divalent cations or combinations thereof

5. The method of claim 4, wherein the divalent cations comprise: Mg²⁺, Mn²⁺, Zn²⁺, Fe²⁺, Ca²⁺, Cu²⁺ or combinations thereof

6. The method of claim 1, wherein a sequence-specific nuclease comprises Clustered Regularly Interspaced Short Palindromic Repeat (CRISPR)-associated endonucleases or homologs thereof, endonucleases, exo-nucleases, Argonautes, restriction enzymes, zinc-finger nucleases (ZFNs), transcription activator-like effector nucleases (TALEN5) meganucleases, or combinations thereof.

7. The method of claim 6, wherein the CRISPR-associated endonucleases comprise: Cas3, Cas4, Cas5, Cas5e (or CasD), Cash, Cas6e, Cas6f, Cas7, Cas8a1, Cas8a2, Cas8b, Cas8c, Cas9, Cas10, CaslOd, CasF, CasG, CasH, CjCas9, SpCas9, Cas13, Cas14, Cpf1, Csyl, Csy2, Csy3, Cse1 (or CasA), Cse2 (or CasB), Cse3 (or CasE), Cse4 (or CasC), Csc1,Csc2, Csa5, Csn2, Csm2, Csm3, Csm4, Csm5, Csm6, Cmr1, Cmr3, Cmr4, Cmr5, Cmr6, Csb1,Csb2, Csb3, Csx17, Csx14, Csx10, Csx16, CsaX, Csx3, Csz1, Csx15, Csf1, Csf2, Csf3, Csf4, Cu1966 or combinations thereof.

8. The method of claim 6, wherein the CRISPR-associated endonuclease is guided to the nucleic acid sequence of interest by at least one guide RNA (gRNA) wherein the gRNA is complementary to a target nucleic acid sequence of the nucleic acid of interest.

9. (canceled)

10. The method of claim 1, wherein the isothermally amplified product is split into two equal volumes, a first volume {A} and a second volume {B}.

11. The method of claim 10, wherein a master mix is added to both volumes {A} and {B}.

12. The method of claim 11, wherein the master mix comprises: (1) a sequence-specific nuclease and (2) a double-stranded DNA exonuclease.

13-23. (canceled)

24. A method of detecting a nucleic acid of interest in a sample, comprising:
preparing primers complementary to the nucleic acid of interest wherein the primers are protected against nuclease activity;
performing isothermal amplification to amplify small amounts of either RNA or DNA sequences from the sample to double stranded DNA (dsDNA);
subjecting the amplified nucleic acid of interest to a gene editing agent; and,
detecting the nucleic acid of interest.

25. The method of claim 24, wherein the primers comprise phosphorothiolate bonds at the 5′ or 3′ end to protect against exonuclease activity.

26. The method of claim 24, wherein the nuclease activity is inhibited by exonuclease inhibitors comprising: citrate, citrate acid; MES, 2-morpholin-4-ylethanesulfonate; PV6R, pontacyl violet 6R; PCMPS, p-chloromercuriphenyl sulfonate; NCA, 7-nitroindole-2-carboxylic acid; DR396, 4-[(4,6-dichloro-1,3,5-triazin-2-yl)amino]-2-(3-hydroxy-6-oxoxanthen-9-yl)benzoic acid; DTNB, 5,5′-dithiobis(2-nitrobenzoic acid); ATA, aurintricarboxylic acid; FDCO, fmoc-D-Cha-OH, Mirin, PFM01/SML1735, PFM03, PFM39/SML1839 or combinations thereof.

27. The method of claim 24, wherein the gene editing agent comprises: CRISPR-associated endonuclease/Cas or Cpf1, Argonaute family of endonucleases, clustered regularly interspaced short palindromic repeat (CRISPR) nucleases, restriction enzymes, zinc-finger nucleases (ZFNs), transcription activator-like effector nucleases (TALEN5), meganucleases, endo- or exo-nucleases, or combinations thereof.

28. The method of claim 24, wherein the gene editing agent is guided to the nucleic acid sequence of interest by at least one guide RNA (gRNA) wherein the gRNA is complementary to a target nucleic acid sequence of the nucleic acid of interest.

29. The method of claim 24, wherein the isothermally amplified product is split into two equal volumes, a first volume {A} and a second volume {B}.

30-43. (anceled)

44. A method of detecting exogenous or abnormal nucleic acid sequences in a subject, comprising the method of claim 1.

45-47. (canceled)

48. A method of treating a subject comprising diagnosing a disease state by detecting an exogenous or abnormal nucleic acid sequences in a subject, comprising the method of claim 1;
administering to the subject a therapy for the diagnosed disease state; and, treating the subject for the diagnosed disease state.

49. A method of detecting SARS-CoV-2 in a biological sample, comprising the method of claim 1.

50-54. (canceled)
